(19) United States
(12) Patent Application Publication
(10) Pub. No.: US 2022/0106275 A1
(43) Pub. Date: Apr. 7, 2022

(54) RADIOLABELED COMPOUNDS AND USES THEREOF

(71) Applicants: The Trustees of Columbia University in the City of New York, New York, NY (US); The Research Foundation for Mental Hygiene, Inc., New York, NY (US)

(72) Inventors: J.S. Dileep KUMAR, Fort Lee, NJ (US); Akiva MINTZ, Paramus, NJ (US); Joseph John MANN, Bronx, NY (US)

(21) Appl. No.: 17/553,462

(22) Filed: Dec. 16, 2021

Related U.S. Application Data
(63) Continuation of application No. PCT/US2020/038739, filed on Jun. 19, 2020.
(60) Provisional application No. 62/864,339, filed on Jun. 20, 2019.

(51) Int. Cl. C07D 239/42; C07D 417/12; C07D 409/12; C07D 261/08; C07D 413/04

(52) U.S. Cl. CPC C07D 239/42; C07D 417/12; C07D 409/12; A61K 51/041; C07D 413/04; C07B 2200/05; C07D 261/08

(57) ABSTRACT

Radiolabeled compounds of Formula I are described. Processes for radiolabeling the compounds are described. Methods for radioactive imaging using the compounds are also described.

### CROSS-REFERENCE TO RELATED APPLICATIONS

This application is a continuation of International Application No. PCT/US2020/038739, filed Jun. 19, 2020, which claims priority to U.S. Provisional Patent Application No. 62/864,339, filed Jun. 20, 2019, the entire contents of each of which are hereby incorporated by reference.

### INCORPORATION BY REFERENCE

All patents, patent applications, and publications cited herein are hereby incorporated by reference in their entirety in order to more fully describe the state of the art as known to those skilled therein as of the date of the invention described herein.

### FIELD OF THE INVENTION

The present disclosure relates generally to the field of radiolabeled compounds. More particularly, the present disclosure relates to radiolabeled arylpyrimidine and isoxazole compounds.

### BACKGROUND

Cyclooxygenase (“COX”) enzymes, also called prostaglandin endoperoxidase synthases, are essential enzymes in prostaglandin biosynthesis. COX enzymes catalyze the oxidative conversion of arachidonic acid substrates to prostaglandins, prostacyclins, and thromboxane. There are three known isoforms of COX: COX-1, COX-2, and COX-3. Among these isoforms, COX-1 is predominantly constitutive. COX-2 is predominantly inducible, though it can also be constitutive in, for example, kidney, brain, heart, liver, testicles, and tracheal epithelia. While the structures of COX-1 and COX-2 are similar, COX-2 is more rapidly degraded, has a shorter half-life, and possesses a larger binding site due to a secondary internal pocket. The third isoform, COX-3, may be responsible for the antipyretic and analgesic activities of non-steroidal anti-inflammatory drugs (“NSAIDs”). COX-1 shares 63% homology with COX-2. COX-3 shares 90% homology with COX-1 and about 60% homology with COX-2.

COX-2 can be induced by inflammatory stimuli and catalyze prostanoid formation associated with inflammation and proliferative diseases. In the central nervous system (“CNS”), COX-2 is modestly expressed under normal physiologic conditions and has a role in, for example, brain, cardiac, and kidney functions. However, during inflammation, COX-2 is significantly upregulated. Neuroinflammation and COX-2 induction are protective under some circumstances, but excessive inflammation and COX-2 induction may be involved in the pathogenesis of disease. These include, for example, inflammation, pain, fever, major depressive disorder (“MDD”), schizophrenia, arthritis (e.g., rheumatoid arthritis and osteoarthritis), neurodegenerative diseases (e.g., Alzheimer's disease, Parkinson's disease, and amyotrophic lateral sclerosis (“ALS”)), brain tumors (see, e.g., Patti et al. Cancer Letters 2002, 180:13-21; hereby incorporated by reference in its entirety), angiogenesis, cancer, stroke, myocardial infarction (see, e.g., Yasojima et al. Brain Research 1999, 830:226-36; hereby incorporated by reference in its entirety), atherosclerosis, diabetes, allograft rejection (see, e.g., Yang et al. Circulation 2000, 101:430-8; hereby incorporated by reference in its entirety), urogenital disease, renal function, tissue repair, bone metabolism, ovulation, pregnancy, child birth, and traumatic brain injury (“TBI”).

Inhibition of COX-2 may be a potential protective treatment strategy by slowing or halting the progression of disease. Monitoring in vivo changes in COX-2 expression could be useful for quantifying disease pathogenesis, such as detecting organ rejection and joints affected by arthritis, and assessing target occupancy and biological effects of COX-2-selective inhibitors (“COXIBs”). Availability of noninvasive diagnostic methods, such as positron emission tomography (“PET”) imaging, is particularly crucial for CNS diseases, given the difficulty of accessing the brain, and presents a direct way of imaging changes in COX-2 in vivo during disease progression and therapy.

Noninvasive PET imaging of COX-2 protein can be a powerful molecular imaging method for quantifying inflammation. A specific COX-2 PET tracer could also serve as a biological marker of COX-2 induction, and as an index of the effect of COXIBs. However, existing radioligands for PET are not effective for imaging COX-2 induction in vivo, possibly due to their suboptimal pharmacology and physiological properties, and other issues in translating to the clinic. For example, [¹¹C]-6-methoxy-2-(4-(methylsulfonyl)phenyl)-N-(thiophen-2-ylmethyl)pyrimidin-4-amine (“[¹¹C]MC1”), a COXIB, showed increased binding to COX-2 during neuroinflammation. However, due to its 20-minute half-life, [¹¹C]MC1 is not practical for multicenter trials, commercial use, or clinical applications, because this half-life is too short to allow the tracer to be shipped beyond the confines of a single medical center. Other ¹¹C-labeled COXIBs (e.g., [¹¹C]celecoxib, [¹¹C]etoricoxib, [¹¹C]rofecoxib, [¹¹C]-3-(4-methylsulfonylphenyl)-4-phenyl-5-trifluoromethyl isoxazole (“[¹¹C]-TMI”), and [¹¹C]-4-[5-(4-methylphenyl)-3-(trifluoromethyl)-1H-pyrazol-1-yl]benzenesulfonamide (“[¹¹C]MOV”)) have been imaged in, for example, mouse, monkey, and baboon, but with issues similar to [¹¹C]MC1. See, e.g., Bioorg. Med. Chem. Lett. 2018, 28:3592-5; Bioorg. Med. Chem. Lett. 2018, 28:2432-5; Molecules 2018, 23:E1929; Bioorg. Med. Chem. 2007, 15:1802-7; Bioorg. Med. Chem. Lett. 2005, 15:4699-702; Bioorg. Med. Chem. Lett. 2005, 15:4268-71; and J. Labeled Comp. Radiopharm. 2005, 48:887-95; each of which is hereby incorporated by reference in its entirety.

Thus, there remains a need for COX-2-selective PET tracers, including those useful for imaging COX-2 expression.

### SUMMARY OF THE INVENTION

In one aspect, a compound of Formula I:

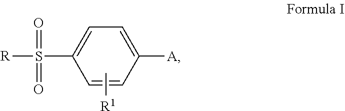

or a pharmaceutically acceptable salt thereof, is described, wherein:

A is

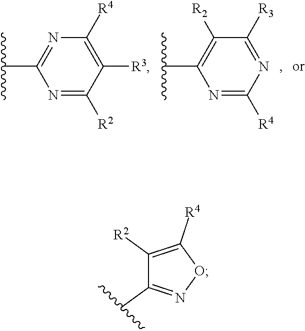

R is selected from the group consisting of (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, (C₁-C₆)alkyl-heteroaryl, and N(R⁵)₂;
wherein each (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, or (C₁-C₆)alkyl-heteroaryl is optionally substituted with one or more deuterium, ³H, ¹¹C, halogen, ¹⁸F, ⁷⁵Br, ⁷⁶Br, ¹²⁰I, ¹²³I, ¹²⁴I, ¹²⁵I, ¹³¹I, N(R⁵)₂, CN, OR⁵, SR⁵, SOR⁵, SO₂R⁵, SO₂NHR⁵, SO₃R⁵, NHSO₂R⁵, COR⁵, or NHCOR⁵;

R¹, R², R³, and R⁴ are each independently selected from the group consisting of hydrogen, deuterium, ³H, ¹¹C, halogen, ¹⁸F, ⁷⁵Br, ⁷⁶Br, ¹²⁰I, ¹²³I, ¹²⁴I, ¹²⁵I, ¹³¹I, (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, (C₁-C₆)alkyl-heteroaryl, N(R⁵)₂, CN, OR⁵, SR⁵, SOR⁵, SO₂R⁵, SO₂NHR⁵, SO₃R⁵, NHSO₂R⁵, COR⁵, and NHCOR⁵;
wherein each (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, or (C₁-C₆)alkyl-heteroaryl is optionally substituted with one or more deuterium, ³H, ¹¹C, halogen, ¹⁸F, ⁷⁵Br, ⁷⁶Br, ¹²⁰I, ¹²³I, ¹²⁴I, ¹²⁵I, ¹³¹I, N(R⁵)₂, CN, OR⁵, SR⁵, SOR⁵, SO₂R⁵, SO₂NHR⁵, SO₃R⁵, NHSO₂R⁵, COR⁵, or NHCOR⁵; and

each occurrence of R⁵ is independently selected from the group consisting of hydrogen, deuterium, ³H, (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, and (C₁-C₆)alkyl-heteroaryl;
wherein each (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, and (C₁-C₆)alkyl-heteroaryl is optionally substituted with one or more deuterium, ³H, ¹¹C, ¹⁸F, ⁷⁵Br, ⁷⁶Br, ¹²⁰I, ¹²³I, ¹²⁴I, ¹²⁵I, or ¹³¹I;
wherein at least one of R, R¹, R², R³, R⁴, or R⁵ comprises at least one of ³H, ¹¹C, ¹⁸F, ⁷⁵Br, ⁷⁶Br, ¹²⁰I, ¹²³I, ¹²⁴I, ¹²⁵I, or ¹³¹I;
wherein when A is

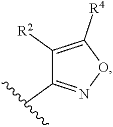

R is ¹¹CH₃, R¹ is hydrogen, and R² is phenyl, R⁴ is not CF₃;
wherein when A is

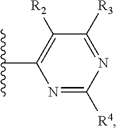

R is CH₃, R¹ and R² are hydrogen, and R³ is CF₃, R⁴ is not

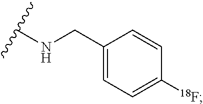

and
wherein when A is

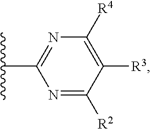

R is CH₃, R¹ and R³ are hydrogen, and R⁴ is O¹¹CH₃, R² is not

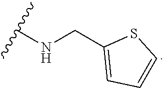 [SMILES: *NCc1cccs1]

In any one of the embodiments described herein, A is

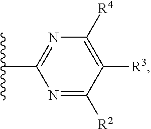

In any one of the embodiments described herein, A is

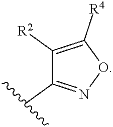

In any one of the embodiments described herein, A is

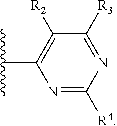

In any one of the embodiments described herein, R is selected from the group consisting of (C₁-C₆)alkyl, (C₁-C₆)alkynyl, (C₁-C₆)heteroalkyl, (C₁-C₆)heteroalkynyl, (C₃-C₇)cycloalkyl, (C₃-C₇)heterocycloalkyl, (C₁-C₆)alkyl-(C₃-C₇)cycloalkyl, (C₁-C₆)alkyl-(C₃-C₇)heterocycloalkyl, aryl, (C₁-C₆)alkyl-aryl, heteroaryl, (C₁-C₆)alkyl-heteroaryl, and N(R⁵)₂;
wherein each (C₁-C₆)alkyl, (C₁-C₆)alkynyl, (C₁-C₆)heteroalkyl, (C₁-C₆)heteroalkynyl, (C₃-C₇)cycloalkyl, (C₃-C₇)heterocycloalkyl, (C₁-C₆)alkyl-(C₃-C₇)cycloalkyl, (C₁-C₆)alkyl-(C₃-C₇)heterocycloalkyl, aryl, (C₁-C₆)alkyl-aryl, heteroaryl, or (C₁-C₆)alkyl-heteroaryl is optionally substituted with deuterium, ¹¹C, ¹⁸F, or ¹²³I.

In any one of the embodiments described herein, each occurrence of R⁵ is independently selected from the group consisting of hydrogen, (C₁-C₆)alkyl, aryl, (C₁-C₆)alkyl-aryl, heteroaryl, and (C₁-C₆)alkyl-heteroaryl;
wherein each (C₁-C₆)alkyl, aryl, (C₁-C₆)alkyl-aryl, heteroaryl, or (C₁-C₆)alkyl-heteroaryl is optionally substituted with deuterium, ¹¹C, ¹⁸F, or ¹²³I.

In any one of the embodiments described herein, R is CH₃, ¹¹CH₃, CH₂¹⁸F, (CH₂)₂¹⁸F, CF₃, CF₂¹⁸F, CD₂¹⁸F,

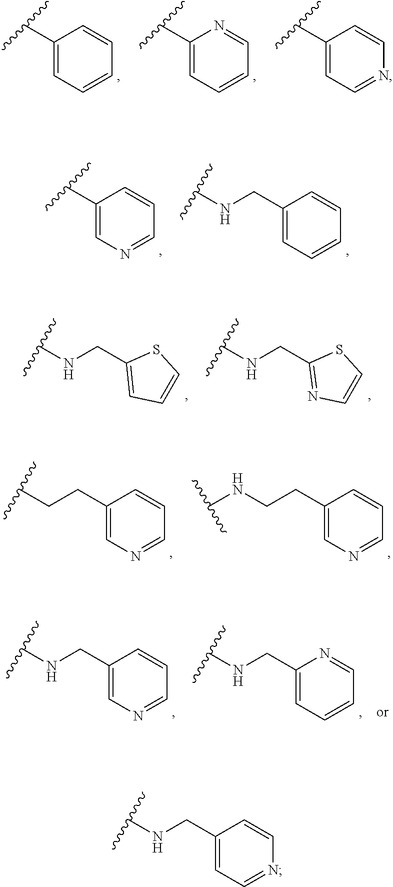

wherein each

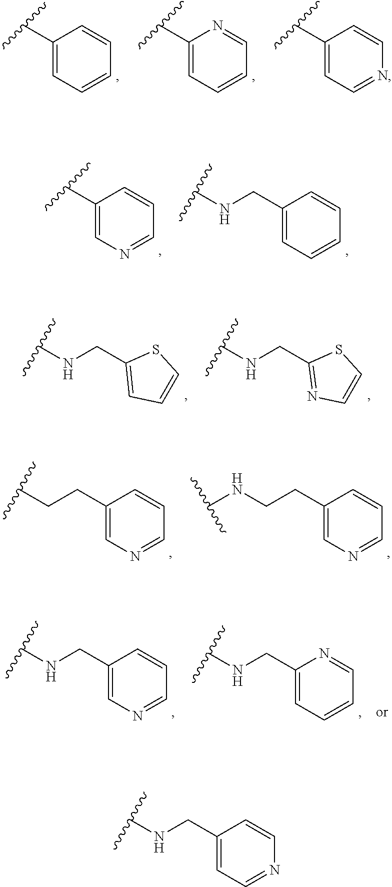

is optionally substituted with deuterium, ¹¹C, ¹⁸F, or ¹²³I.

In any one of the embodiments described herein, R is CH₃, ¹¹CH₃, or (CH₂)₂¹⁸F.

In any one of the embodiments described herein, R¹, R², R³, and R⁴ are each independently selected from the group consisting of hydrogen, deuterium, halogen, ¹¹C, ¹⁸F, ¹²³I, (C₁-C₆)alkyl, (C₁-C₆)alkynyl, (C₁-C₆)heteroalkyl, (C₁-C₆)heteroalkynyl, (C₃-C₇)cycloalkyl, (C₃-C₇)heterocycloalkyl, (C₁-C₆)alkyl-(C₃-C₇)cycloalkyl, (C₁-C₆)alkyl-(C₃-C₇)heterocycloalkyl, aryl, (C₁-C₆)alkyl-aryl, heteroaryl, (C₁-C₆)alkyl-heteroaryl, and N(R⁵)₂;
wherein each (C₁-C₆)alkyl, (C₁-C₆)alkynyl, (C₁-C₆)heteroalkyl, (C₁-C₆)heteroalkynyl, (C₃-C₇)cycloalkyl, (C₃-C₇)heterocycloalkyl, (C₁-C₆)alkyl-(C₃-C₇)cycloalkyl, (C₁-C₆)alkyl-(C₃-C₇)heterocycloalkyl, aryl, (C₁-C₆)alkyl-aryl, heteroaryl, or (C₁-C₆)alkyl-heteroaryl is optionally substituted with deuterium, ¹¹C, ¹⁸F, or ¹²³I.

In any one of the embodiments described herein, each occurrence of R⁵ is independently selected from the group consisting of hydrogen, deuterium, (C₁-C₆)alkyl, aryl, (C₁-C₆)alkyl-aryl, heteroaryl, and (C₁-C₆)alkyl-heteroaryl;
wherein each (C₁-C₆)alkyl, aryl, (C₁-C₆)alkyl-aryl, heteroaryl, or (C₁-C₆)alkyl-heteroaryl is optionally substituted with deuterium, ¹¹C, ¹⁸F, or ¹²³I.

In any one of the embodiments described herein, R¹, R², R³, and R⁴ are each independently hydrogen, deuterium, halogen, ¹⁸F, ¹²³I, CH₃, ¹¹CH₃, CH₂¹⁸F, (CH₂)₂¹⁸F, CF₃, CF₂¹⁸F, CD₂¹⁸F,

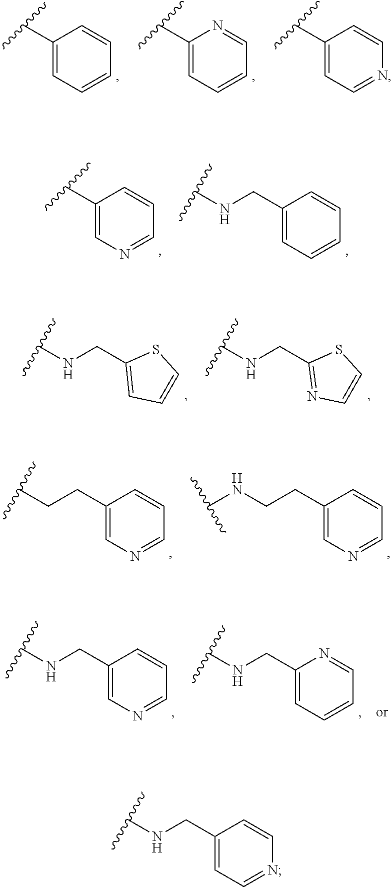

wherein each

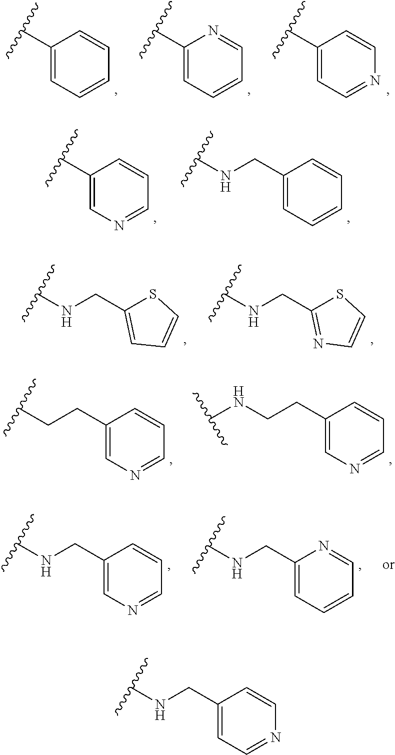

is optionally substituted with deuterium, ¹¹C, ¹⁸F, or ¹²³I.

In any one of the embodiments described herein, R¹ is hydrogen or ¹⁸F.

In any one of the embodiments described herein, R² is hydrogen,

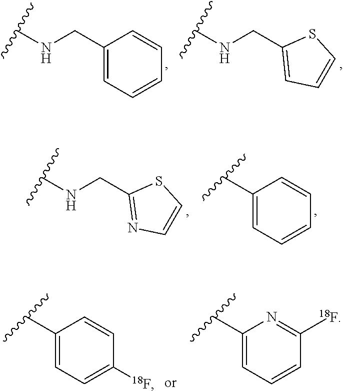

In any one of the embodiments described herein, R³ is hydrogen or ¹⁸F.

In any one of the embodiments described herein, R⁴ is halogen, ¹⁸F, CF₂¹⁸F, CH₂¹⁸F, CD₂¹⁸F, CF₃,

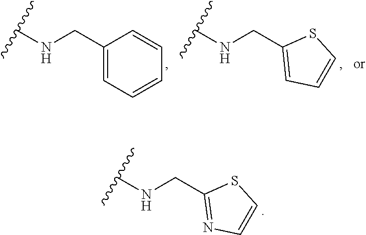

In any one of the embodiments described herein,

A is

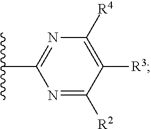

R is CH₃ or ¹¹CH₃;

R¹ and R³ are hydrogen;

R² is N(R⁵)₂;

R⁴ is F, Cl, CF₃, or ¹⁸F; and

each occurrence of R⁵ is independently hydrogen, (C₁-C₆)alkyl-aryl, or (C₁-C₆)alkyl-heteroaryl.

In any one of the embodiments described herein, each occurrence of R⁵ is independently hydrogen,

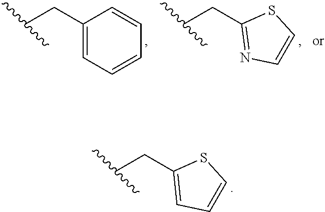

In any one of the embodiments described herein, R² is

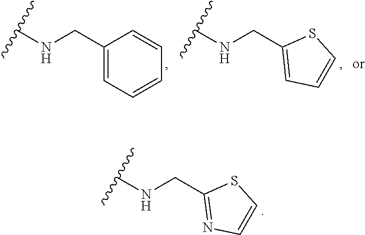

In any one of the embodiments described herein,

A is

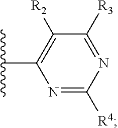

R is CH₃;

R¹ and R² are hydrogen;

R² is ¹⁸F;

R⁴ is (NR⁵)₂; and

each occurrence of R⁵ is independently hydrogen, (C₁-C₆)alkyl-aryl, or (C₁-C₆)alkyl-heteroaryl.

In any one of the embodiments described herein,

A is

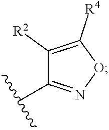

R and R⁴ are (C₁-C₆)alkyl, optionally substituted with one or more deuterium, ¹⁸F, or halogen;

R¹ is hydrogen or ¹⁸F; and

R² is aryl, optionally substituted with ¹⁸F, or heteroaryl, optionally substituted with ¹⁸F.

In any one of the embodiments described herein, R is CHs or (CH₂)₂¹⁸F.

In any one of the embodiments described herein, R² is

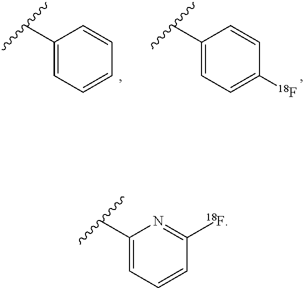

In any one of the embodiments described herein, R⁴ is CF₃, CF₂¹⁸F, CH₂¹⁸F, or CD₂¹⁸F.

In any one of the embodiments described herein, the compound of Formula I is

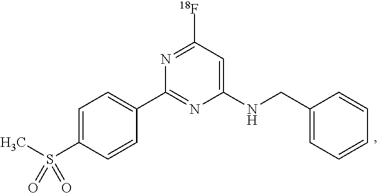 [SMILES: CS(=O)(=O)c1ccc(-c2nc([18F])cc(NCc3ccccc3)n2)cc1]

or a pharmaceutically acceptable salt thereof.

In any one of the embodiments described herein, the compound of Formula I is

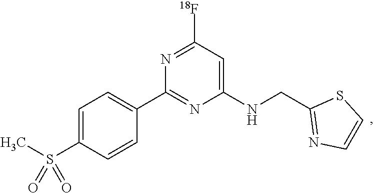 [SMILES: CS(=O)(=O)c1ccc(-c2nc([18F])cc(NCc3nccs3)n2)cc1]

or a pharmaceutically acceptable salt thereof.

In any one of the embodiments described herein, the compound of Formula I is

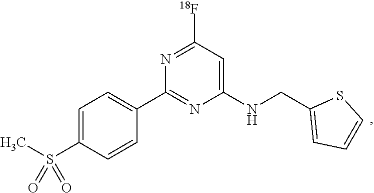 [SMILES: CS(=O)(=O)c1ccc(-c2nc([18F])cc(NCc3cccs3)n2)cc1]

or a pharmaceutically acceptable salt thereof.

In any one of the embodiments described herein, the compound of Formula I is

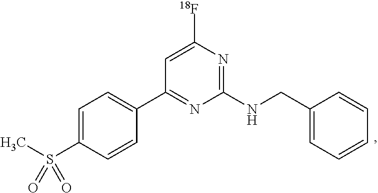 [SMILES: CS(=O)(=O)c1ccc(-c2cc([18F])nc(NCc3ccccc3)n2)cc1]

or a pharmaceutically acceptable salt thereof.

In any one of the embodiments described herein, the compound of Formula I is

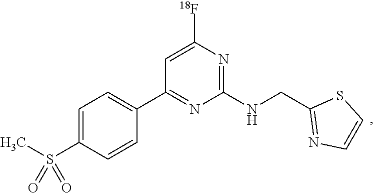 [SMILES: CS(=O)(=O)c1ccc(-c2cc([18F])nc(NCc3nccs3)n2)cc1]

or a pharmaceutically acceptable salt thereof.

In any one of the embodiments described herein, the compound of Formula I is

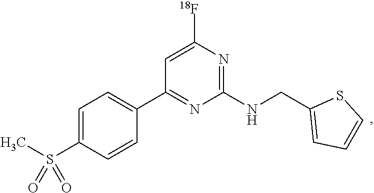 [SMILES: CS(=O)(=O)c1ccc(-c2cc([18F])nc(NCc3cccs3)n2)cc1]

or a pharmaceutically acceptable salt thereof.

In any one of the embodiments described herein, the compound of Formula I is

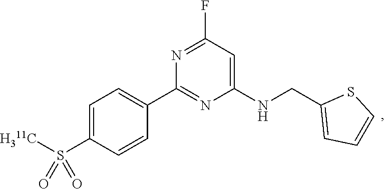 [SMILES: [11CH3]S(=O)(=O)c1ccc(-c2nc(F)cc(NCc3cccs3)n2)cc1]

or a pharmaceutically acceptable salt thereof.

In any one of the embodiments described herein, the compound of Formula I is

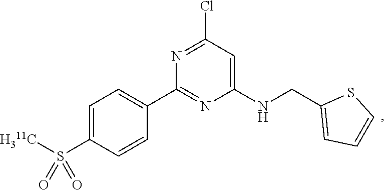 [SMILES: [11CH3]S(=O)(=O)c1ccc(-c2nc(Cl)cc(NCc3cccs3)n2)cc1]

or a pharmaceutically acceptable salt thereof.

In any one of the embodiments described herein, the compound of Formula I is

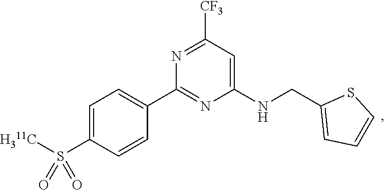 [SMILES: [11CH3]S(=O)(=O)c1ccc(-c2nc(NCc3cccs3)cc(C(F)(F)F)n2)cc1]

or a pharmaceutically acceptable salt thereof.

In any one of the embodiments described herein, the compound of Formula I is

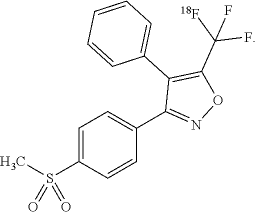 [SMILES: CS(=O)(=O)c1ccc(-c2noc(C(F)(F)[18F])c2-c2ccccc2)cc1]

In any one of the embodiments described herein, the compound of Formula I is

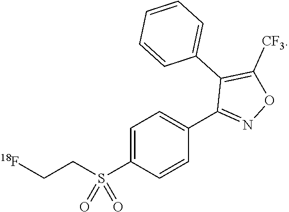 [SMILES: O=S(=O)(CC[18F])c1ccc(-c2noc(C(F)(F)F)c2-c2ccccc2)cc1]

In any one of the embodiments described herein, the compound of Formula I is

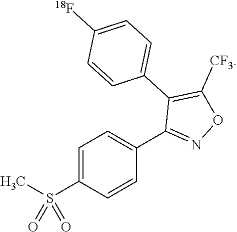 [SMILES: CS(=O)(=O)c1ccc(-c2noc(C(F)(F)F)c2-c2ccc([18F])cc2)cc1]

In any one of the embodiments described herein, the compound of Formula I is

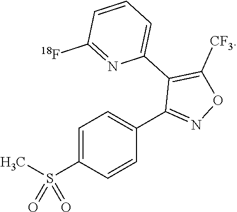 [SMILES: CS(=O)(=O)c1ccc(-c2noc(C(F)(F)F)c2-c2cccc([18F])n2)cc1]

In any one of the embodiments described herein, the compound of Formula I is

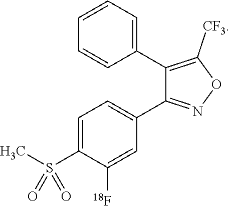 [SMILES: CS(=O)(=O)c1ccc(-c2noc(C(F)(F)F)c2-c2ccccc2)cc1[18F]]

In any one of the embodiments described herein, the compound of Formula I is

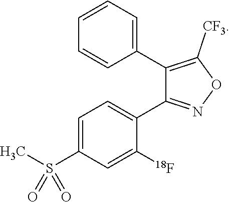 [SMILES: CS(=O)(=O)c1ccc(-c2noc(C(F)(F)F)c2-c2ccccc2)c([18F])c1]

In any one of the embodiments described herein, the compound of Formula I is

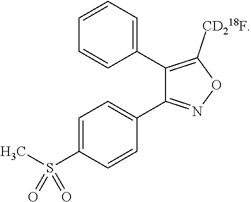

In any one of the embodiments described herein, the compound of Formula I is

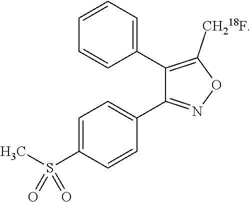

In another aspect, a process for synthesizing a compound of Formula I:

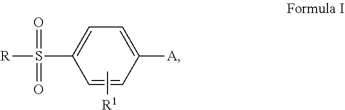

or a pharmaceutically acceptable salt thereof, is described,

comprising reacting one or more halogen atom(s) or thioester(s) in the compound with one or more radionuclide source(s) independently selected from the group consisting of deuterium, ³H, ¹¹C, ¹⁸F, ⁷⁵Br, ⁷⁶Br, ¹²⁰I, ¹²³I, ¹²⁴I, ¹²⁵I, and ¹³¹I;

wherein:

A is

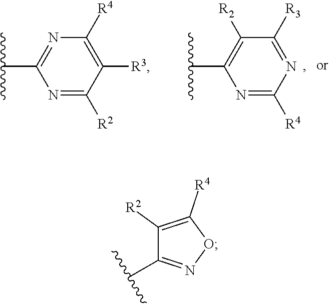

R is selected from the group consisting of (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, (C₁-C₆)alkyl-heteroaryl, and N(R⁵)₂;
wherein each (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, or (C₁-C₆)alkyl-heteroaryl is optionally substituted with one or more deuterium, ³H, ¹¹C, halogen, ¹⁸F, ⁷⁵Br, ⁷⁶Br, ¹²⁰I, ¹²³I, ¹²⁴I, ¹²⁵I, ¹³¹I, N(R⁵)₂, CN, OR⁵, SR⁵, SOR⁵, SO₂R⁵, SO₂NHR⁵, SO₃R⁵, NHSO₂R⁵, COR⁵, or NHCOR⁵;

R¹, R², R³, and R⁴ are each independently selected from the group consisting of hydrogen, deuterium, ³H, ¹¹C, halogen, ¹⁸F, ⁷⁵Br, ⁷⁶Br, ¹²⁰I, ¹²³I, ¹²⁴I, ¹²⁵I, ¹³¹I, (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, (C₁-C₆)alkyl-heteroaryl, N(R⁵)₂, CN, OR⁵, SR⁵, SOR⁵, SO₂R⁵, SO₂NHR⁵, SO₃R⁵, NHSO₂R⁵, COR⁵, and NHCOR⁵;
wherein each (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, or (C₁-C₆)alkyl-heteroaryl is optionally substituted with one or more deuterium, ³H, ¹¹C, halogen, ¹⁸F, ⁷⁵Br, ⁷⁶Br, ¹²⁰I, ¹²³I, ¹²⁴I, ¹²⁵I, ¹³¹I, N(R⁵)₂, CN, OR⁵, SR⁵, SOR⁵, SO₂R⁵, SO₂NHR⁵, SO₃R⁵, NHSO₂R⁵, COR⁵, or NHCOR⁵; and

each occurrence of R⁵ is independently selected from the group consisting of hydrogen, deuterium, ³H, (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, and (C₁-C₆)alkyl-heteroaryl;
wherein each (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, and (C₁-C₆)alkyl-heteroaryl is optionally substituted with one or more deuterium, ³H, ¹¹C, ¹⁸F, ⁷⁵Br, ⁷⁶Br, ¹²⁰I, ¹²³I, ¹²⁴I, ¹²⁵I, or ¹³¹I;

wherein at least one of R, R¹, R², R³, R⁴, or R⁵ comprises at least one of ³H, ¹¹C, ¹⁸F, ⁷⁵Br, ⁷⁶Br, ¹²⁰I, ¹²³I, ¹²⁴I, ¹²⁵I, or ¹³¹I;

wherein when A is

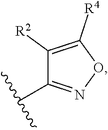

R is ¹¹CH₃, R¹ is hydrogen, and R² is phenyl, R⁴ is not CF₃;

wherein when A is

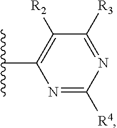

R is CH₃, R¹ and R² are hydrogen, and R³ is CF₃, R⁴ is not

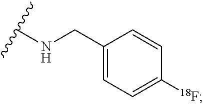

and

wherein when A is

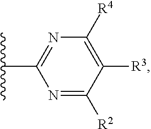

R is CH₃, R¹ and R³ are hydrogen, and R⁴ is O¹¹CH₃, R² is not

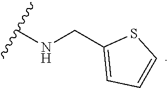 [SMILES: *NCc1cccs1]

In any one of the embodiments described herein, the one or more halogen atom(s) are independently chlorine or bromine.

In any one of the embodiments described herein, the radionuclide source is a ¹⁸F source.

In any one of the embodiments described herein, the one or more thioester(s) comprise

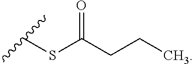 [SMILES: *SC(=O)CCC]

In any one of the embodiments described herein, the radionuclide source is a ¹¹C source and wherein the process further comprises an oxidant.

In any one of the embodiments described herein, the compound of Formula I is

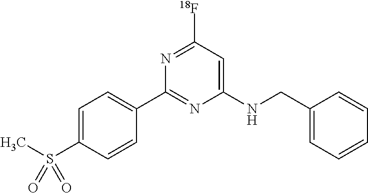 [SMILES: CS(=O)(=O)c1ccc(-c2nc([18F])cc(NCc3ccccc3)n2)cc1]

or a pharmaceutically acceptable salt thereof; comprising reacting the compound

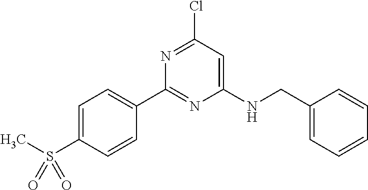 [SMILES: CS(=O)(=O)c1ccc(-c2nc(Cl)cc(NCc3ccccc3)n2)cc1]

with a ¹⁸F source.

In any one of the embodiments described herein, the compound of Formula I is

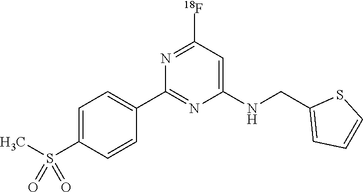 [SMILES: CS(=O)(=O)c1ccc(-c2nc([18F])cc(NCc3cccs3)n2)cc1]

or a pharmaceutically acceptable salt thereof; comprising reacting the compound

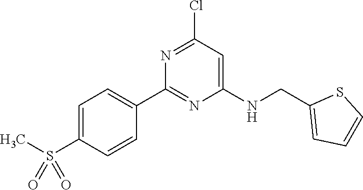 [SMILES: CS(=O)(=O)c1ccc(-c2nc(Cl)cc(NCc3cccs3)n2)cc1]

with a ¹⁸F source.

In any one of the embodiments described herein, the compound of Formula I is

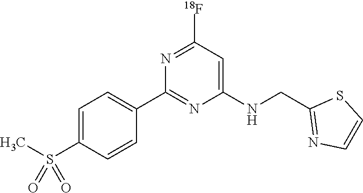 [SMILES: CS(=O)(=O)c1ccc(-c2nc([18F])cc(NCc3nccs3)n2)cc1]

or a pharmaceutically acceptable salt thereof; comprising reacting the compound

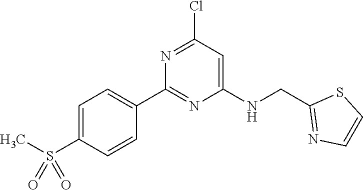 [SMILES: CS(=O)(=O)c1ccc(-c2nc(Cl)cc(NCc3nccs3)n2)cc1]

with a ¹⁸F source.

In any one of the embodiments described herein, the compound of Formula I is

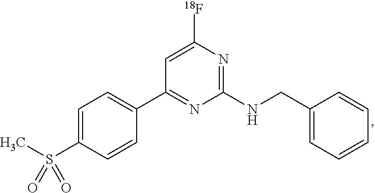 [SMILES: CS(=O)(=O)c1ccc(-c2cc([18F])nc(NCc3ccccc3)n2)cc1]

or a pharmaceutically acceptable salt thereof; comprising reacting the compound

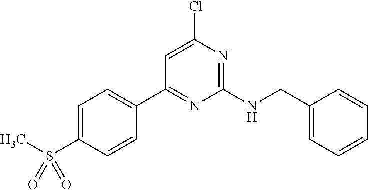 [SMILES: CS(=O)(=O)c1ccc(-c2cc(Cl)nc(NCc3ccccc3)n2)cc1]

with a ¹⁸F source.

In any one of the embodiments described herein, the compound of Formula I is

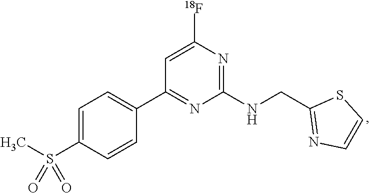 [SMILES: CS(=O)(=O)c1ccc(-c2cc([18F])nc(NCc3nccs3)n2)cc1]

or a pharmaceutically acceptable salt thereof; comprising reacting the compound

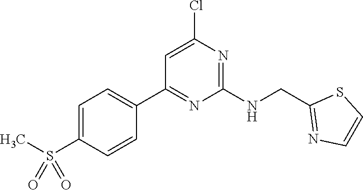 [SMILES: CS(=O)(=O)c1ccc(-c2cc(Cl)nc(NCc3nccs3)n2)cc1]

with a ¹⁸F source.

In any one of the embodiments described herein, the compound of Formula I is

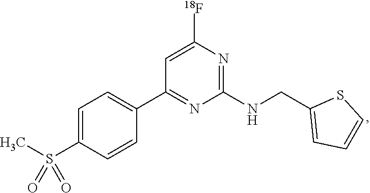 [SMILES: CS(=O)(=O)c1ccc(-c2cc([18F])nc(NCc3cccs3)n2)cc1]

or a pharmaceutically acceptable salt thereof; comprising reacting the compound

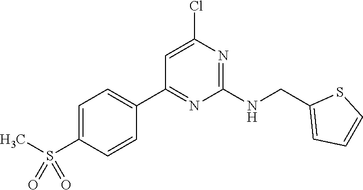 [SMILES: CS(=O)(=O)c1ccc(-c2cc(Cl)nc(NCc3cccs3)n2)cc1]

with a ¹⁸F source.

In any one of the embodiments described herein, the compound of Formula I is

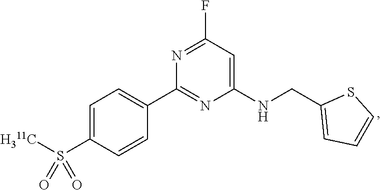 [SMILES: [11CH3]S(=O)(=O)c1ccc(-c2nc(F)cc(NCc3cccs3)n2)cc1]

or a pharmaceutically acceptable salt thereof; comprising reacting the compound

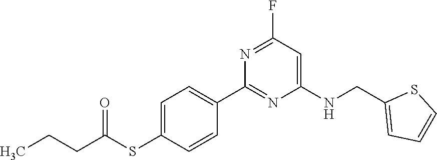 [SMILES: CCCC(=O)Sc1ccc(-c2nc(F)cc(NCc3cccs3)n2)cc1]

with a ¹¹C source and an oxidant.

In any one of the embodiments described herein, the compound of Formula I is

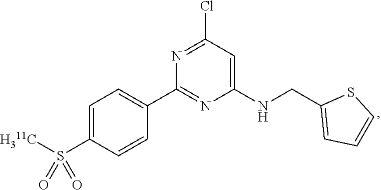 [SMILES: [11CH3]S(=O)(=O)c1ccc(-c2nc(Cl)cc(NCc3cccs3)n2)cc1]

or a pharmaceutically acceptable salt thereof; comprising reacting the compound

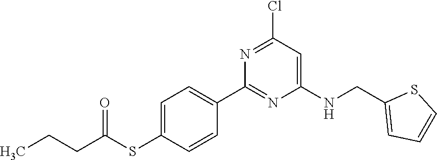 [SMILES: CCCC(=O)Sc1ccc(-c2nc(Cl)cc(NCc3cccs3)n2)cc1]

with a ¹¹C source and an oxidant.

In any one of the embodiments described herein, the compound of Formula I is

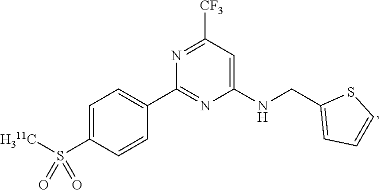 [SMILES: [11CH3]S(=O)(=O)c1ccc(-c2nc(NCc3cccs3)cc(C(F)(F)F)n2)cc1]

or a pharmaceutically acceptable salt thereof; comprising reacting the compound

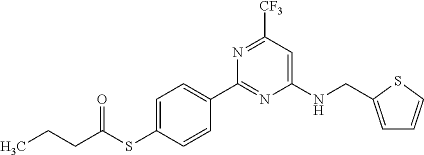 [SMILES: CCCC(=O)Sc1ccc(-c2nc(NCc3cccs3)cc(C(F)(F)F)n2)cc1]

with a ¹¹C source and an oxidant.

In any one of the embodiments described herein, the compound of Formula I is

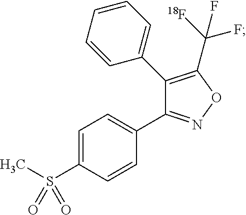 [SMILES: CS(=O)(=O)c1ccc(-c2noc(C(F)(F)[18F])c2-c2ccccc2)cc1]

comprising reacting the compound

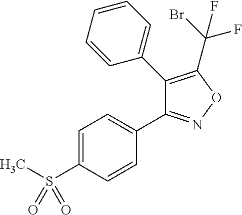 [SMILES: CS(=O)(=O)c1ccc(-c2noc(C(F)(F)Br)c2-c2ccccc2)cc1]

with a ¹⁸F source.

In any one of the embodiments described herein, the compound of Formula I is

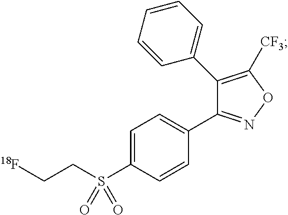 [SMILES: O=S(=O)(CC[18F])c1ccc(-c2noc(C(F)(F)F)c2-c2ccccc2)cc1]

comprising reacting the compound

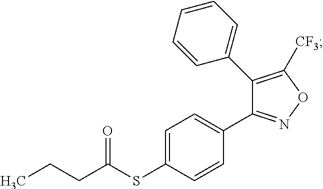 [SMILES: CCCC(=O)Sc1ccc(-c2noc(C(F)(F)F)c2-c2ccccc2)cc1]

with a ¹⁸F source.

In any one of the embodiments described herein, the compound of Formula I is

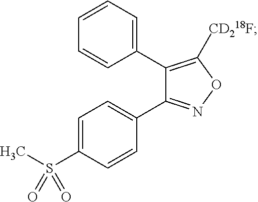

comprising reacting the compound

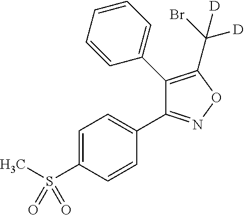 [SMILES: [2H]C([2H])(Br)c1onc(-c2ccc(S(C)(=O)=O)cc2)c1-c1ccccc1]

with a ¹⁸F source.

In any one of the embodiments described herein, the compound of Formula I is

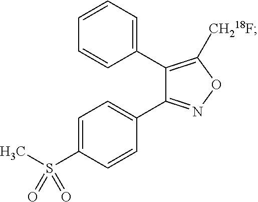

comprising reacting the compound

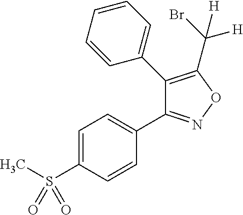 [SMILES: CS(=O)(=O)c1ccc(-c2noc(CBr)c2-c2ccccc2)cc1]

with a ¹⁸F source.

In any one of the embodiments described herein, the ¹⁸F source comprises ¹⁸F or [¹⁸F]fluoroalkyl tosylate.

In any one of the embodiments described herein, the ¹⁸F source comprises K¹⁸F or a tetraalkylammonium [¹⁸F]fluoride.

In any one of the embodiments described herein, the tetraalkylammonium [¹⁸F]fluoride is tetrabutylammonium [¹⁸F]fluoride or tetraethylammonium [¹⁸F]fluoride.

In any one of the embodiments described herein, the [¹⁸F]fluoroalkyl tosylate is 2-[¹⁸F]fluoroethyl tosylate.

In any one of the embodiments described herein, the process further comprises a chelating agent, a base, and/or a polar aprotic solvent.

In any one of the embodiments described herein, the chelating agent is a crown ether or a cryptand.

In any one of the embodiments described herein, the cryptand is K₂₂₂.

In any one of the embodiments described herein, the base is potassium carbonate, tetrabutylammonium hydroxide, pyrrolidine, or 2,6-lutidine.

In any one of the embodiments described herein, the polar aprotic solvent is dimethyl sulfoxide, N,N-dimethylformamide, tetrahydrofuran, or combinations thereof.

In any one of the embodiments described herein, the ¹¹C source comprises a [¹¹C]alkyl halide and a [¹¹C]alkyl triflate.

In any one of the embodiments described herein, the [¹¹C]alkyl halide is ¹¹CH₃I and the [¹¹C]alkyl triflate is ¹¹CH₃OTf.

In any one of the embodiments described herein, the oxidant is Oxone®.

In yet another aspect, a method for imaging a sample or organism comprises:

(a) administering to the sample or organism a compound of Formula I:

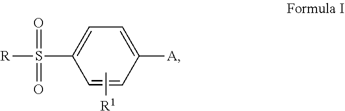

or a pharmaceutically acceptable salt thereof, in an amount effective to detect emission of deuterium, ³H, ¹¹C, ¹⁸F, ⁷⁵Br, ⁷⁶Br, ¹²⁰I, ¹²³I, ¹²⁴I, ¹²⁵I, ¹³¹I, or a combination thereof; and

(b) measuring the emission of the one or more radionuclide(s) in the compound;

wherein:

A is

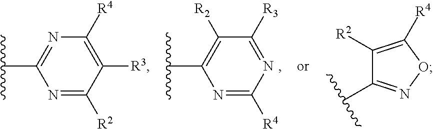

R is selected from the group consisting of (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, (C₁-C₆)alkyl-heteroaryl, and N(R⁵)₂;
wherein each (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, or (C₁-C₆)alkyl-heteroaryl is optionally substituted with one or more deuterium, ³H, ¹¹C, halogen, ¹⁸F, ⁷⁵Br, ⁷⁶Br, ¹²⁰I, ¹²³I, ¹²⁴I, ¹²⁵I, ¹³¹I, N(R⁵)₂, CN, OR⁵, SR⁵, SOR⁵, SO₂R⁵, SO₂NHR⁵, SO₃R⁵, NHSO₂R⁵, COR⁵, or NHCOR⁵;

R¹, R², R³, and R⁴ are each independently selected from the group consisting of hydrogen, deuterium, ³H, ¹¹C, halogen, ¹⁸F, ⁷⁵Br, ⁷⁶Br, ¹²⁰I, ¹²³I, ¹²⁴I, ¹²⁵I, ¹³¹I, (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, (C₁-C₆)alkyl-heteroaryl, N(R⁵)₂, CN, OR⁵, SR⁵, SOR⁵, SO₂R⁵, SO₂NHR⁵, SO₃R⁵, NHSO₂R⁵, COR⁵, and NHCOR⁵;
wherein each (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, or (C₁-C₆)alkyl-heteroaryl is optionally substituted with one or more deuterium, ³H, ¹¹C, halogen, ¹⁸F, ⁷⁵Br, ⁷⁶Br, ¹²⁰I, ¹²³I, ¹²⁴I, ¹²⁵I, ¹³¹I, N(R⁵)₂, CN, OR⁵, SR⁵, SOR⁵, SO₂R⁵, SO₂NHR⁵, SO₃R⁵, NHSO₂R⁵, COR⁵, or NHCOR⁵; and

each occurrence of R⁵ is independently selected from the group consisting of hydrogen, deuterium, ³H, (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, and (C₁-C₆)alkyl-heteroaryl;
wherein each (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, and (C₁-C₆)alkyl-heteroaryl is optionally substituted with one or more deuterium, ³H, ¹¹C, ¹⁸F, ⁷⁵Br, ⁷⁶Br, ¹²⁰I, ¹²³I, ¹²⁴I, ¹²⁵I, or ¹³¹I;

wherein at least one of R, R¹, R², R³, R⁴, or R⁵ comprises at least one of ³H, ¹¹C, ¹⁸F, ⁷⁵Br, ⁷⁶Br, ¹²⁰I, ¹²³I, ¹²⁴I, ¹²⁵I, or ¹³¹I;

wherein when A is

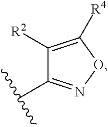

R is ¹¹CH₃, R¹ is hydrogen, and R² is phenyl, R⁴ is not CF₃;

wherein when A is

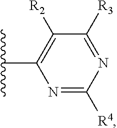

R is CH₃, R¹ and R² are hydrogen, and R³ is CF₃, R⁴ is not

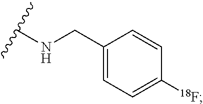

and

wherein when A is

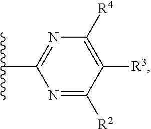

R is CH₃, R¹ and R³ are hydrogen, and R⁴ is O¹¹CH₃, R² is not

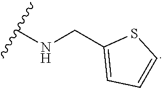 [SMILES: *NCc1cccs1]

In any one of the embodiments described herein, the emission is measured using positron emission tomography.

In any one of the embodiments described herein, the sample is a cell culture.

In any one of the embodiments described herein, the organism is a mammal.

In any one of the embodiments described herein, the mammal is a mouse, rat, pig, dog, monkey, baboon, or human.

In any one of the embodiments described herein, the compound interacts with a protein and measuring the emission allows determination of the expression level or distribution of the protein in the sample or organism.

In any one of the embodiments described herein, the protein is involved in inflammation and measuring the emission allows determination of the amount or distribution of inflammation in the sample or organism.

In any one of the embodiments described herein, the protein is cyclooxygenase-2.

In any one of the embodiments described herein, the mammal is known or suspected to have a condition or state selected from the group consisting of arthritis, cardiovascular disease, central nervous system damage, central nervous system disorders, gastrointestinal disorder, hypersensitivity, swelling, inflammatory disease, metabolic disorder, neoplastic disease, neurodegenerative disease, neuromuscular junction disease, ophthalmic disease, post-operative inflammation, psychiatric condition, reproductive event, respiratory disease, skin disorder, tissue repair, urogenital disease, white matter disease, and a combination thereof.

In any one of the embodiments described herein, the neurodegenerative disease is Alzheimer's disease, Parkinson's disease, Huntington's disease, amyotrophic lateral sclerosis, cortical dementias, or multiple sclerosis.

In any one of the embodiments described herein, the arthritis is rheumatoid or osteoarthritis.

In any one of the embodiments described herein, the neoplastic disease comprises a cancer selected from the group consisting of colorectal cancer, breast cancer, lung cancer, prostate cancer, bladder cancer, cervical cancer, skin cancer, lymphoma, and a combination thereof.

In any one of the embodiments described herein, the central nervous system damage or disorder comprises traumatic brain injury.

In any one of the embodiments described herein, the inflammatory disease is asthma, Bechet's disease, bronchitis, bursitis, Crohn's disease, endotoxin shock syndrome, gastritis, gingivitis, inflammatory bowel disease, polymyositis, pulmonary inflammation, cystic fibrosis, rheumatic fever, sarcoidosis, tendinitis, thyroiditis, or ulcerative colitis.

In any one of the embodiments described herein, the pulmonary inflammation is an acute respiratory disease syndrome resulting from viral and/or bacterial infection.

In any one of the embodiments described herein, the viral infection comprises COVID-19.

In any one of the embodiments described herein, the cardiovascular disease comprises myocardial infarction.

In any one of the embodiments described herein, the urogenital disease comprises renal disease.

In any one of the embodiments described herein, the reproductive event comprises ovulation, pregnancy, childbirth, and a combination thereof.

In any one of the embodiments described herein, the mammal is known or suspected to be experiencing pain.

Any aspect or embodiment disclosed herein may be combined with another aspect or embodiment disclosed herein. The combination of one or more embodiments described herein with other one or more embodiments described herein is expressly contemplated.

Unless otherwise defined, used, or characterized herein, terms that are used herein (including technical and scientific terms) are to be interpreted as having a meaning that is consistent with their accepted meaning in the context of the relevant art and are not to be interpreted in an idealized or overly formal sense unless expressly so defined herein. The terminology used herein is for the purpose of describing particular embodiments and is not intended to be limiting of exemplary embodiments. As used herein, singular forms, such as “a” and “an,” are intended to include the plural forms as well, unless the context indicates otherwise. The term “about” as used herein can describe a range of a recited value, including ±10%, ±5%, or ±2% of the value. Additionally, the terms “includes,” “including,” “comprises,” and “comprising” specify the presence of the stated elements or steps but do not preclude the presence or addition of one or more other elements or steps.

### BRIEF DESCRIPTION OF THE FIGURES

The invention is described with reference to the following figures, which are presented for the purpose of illustration only and are not intended to be limiting. The patent or application file contains at least one drawing executed in color. Copies of this patent or patent application publication with color drawing(s) will be provided by the United States Patent and Trademark Office upon request and payment of the necessary fee.

FIG. 1 shows chemical structures of [¹¹C]MC1, [¹⁸F]pyricoxib, [¹⁸F]triacoxib, and [¹⁸F]-6-fluoro-2-(4-(methylsulfonyl)phenyl)-N-(thiazol-2-ylmethyl)pyrimidin-4-amine (“[¹⁸F]FMTP”), according to one or more embodiments.

FIG. 2 shows binding of [¹⁸F]FMTP in BxPC3 and PANC-1 cells, according to one or more embodiments.

FIG. 3A shows microPET images of [¹⁸F]FMTP in mice at 0-10 minutes, summed transaxial, with the relative intensity of ¹⁸F emission indicated by a red (highest)-orange-yellow-green-blue-black (lowest) color gradient, according to one or more embodiments.

FIG. 3B shows microPET images of [¹⁸F]FMTP in mice at 0-10 minutes, summed sagittal, with the relative intensity of ¹⁸F emission indicated by a red (highest)-orange-yellow-green-blue-black (lowest) color gradient, according to one or more embodiments.

FIG. 3C shows microPET images of [¹⁸F]FMTP in mice at 0-40 minutes, summed transaxial, with the relative intensity of ¹⁸F emission indicated by a red (highest)-orange-yellow-green-blue-black (lowest) color gradient, according to one or more embodiments.

FIG. 3D shows microPET images of [¹⁸F]FMTP in mice at 0-40 minutes, summed sagittal, with the relative intensity of ¹⁸F emission indicated by a red (highest)-orange-yellow-green-blue-black (lowest) color gradient, according to one or more embodiments.

FIG. 4 shows time activity curves of [¹⁸F]FMTP in mice, according to one or more embodiments.

FIG. 5 shows microPET images of [¹⁸F]FMTP in phosphate buffered saline (“PBS”)- and lipopolysaccharide (“LPS”)-treated mice, with the relative intensity of ¹⁸F emission indicated by a red (highest)-orange-yellow-green-blue-black (lowest) color gradient, according to one or more embodiments.

FIG. 6 shows time activity curves of [¹⁸F]FMTP in PBS- and LPS-treated mice, according to one or more embodiments.

FIG. 7 shows autoradiography evaluation of [¹⁸F]FMTP in brain sections of LPS- and PBS-treated mice, according to one or more embodiments.

### DETAILED DESCRIPTION OF THE INVENTION

Radiolabeled compounds that interact with biomarkers (e.g., proteins) for disease are useful for assaying the presence, amount, activity, or other properties of the biomarker using radioactive imaging (e.g., PET). Such imaging can be used to diagnose disease, monitor disease progression and treatment, and determine the lowest effective dose of therapeutics, and evaluate the effectiveness of therapeutics. For example, the COX-2 protein, which is a biomarker for various diseases described herein, can be assayed using PET imaging of a radiolabeled compound that binds or otherwise interacts with COX-2. However, many existing radiolabeled compounds have a half-life that is impractical for medical use. For example, some radiolabeled COX-2-interacting compounds incorporate an ¹¹C label, which has a half-life of about 20 minutes. It is not practical for such compounds to be sold commercially, used in long-term imaging applications, or transported between medical centers.

Compounds

In one aspect, a compound of Formula I

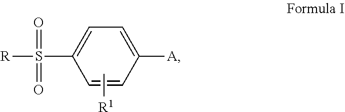

or a pharmaceutically acceptable salt thereof, is described,
wherein

A is

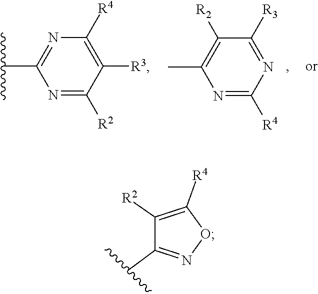

R is selected from the group consisting of (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, (C₁-C₆)alkyl-heteroaryl, and N(R⁵)₂;
wherein each (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, or (C₁-C₆)alkyl-heteroaryl is optionally substituted with one or more deuterium, ³H, ¹¹C, halogen, ¹⁸F, ⁷⁵Br, ⁷⁶Br, ¹²⁰I, ¹²³I, ¹²⁴I, ¹²⁵I, ¹³¹I, N(R⁵)₂, CN, OR⁵, SR⁵, SOR⁵, SO₂R⁵, SO₂NHR⁵, SO₃R⁵, NHSO₂R⁵, COR⁵, or NHCOR⁵;

R¹, R², R³, and R⁴ are each independently selected from the group consisting of hydrogen, deuterium, ³H, ¹¹C, halogen, ¹⁸F, ⁷⁵Br, ⁷⁶Br, ¹²⁰I, ¹²³I, ¹²⁴I, ¹²⁵I, ¹³¹I, (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, (C₁-C₆)alkyl-heteroaryl, N(R⁵)₂, CN, OR⁵, SR⁵, SOR⁵, SO₂R⁵, SO₂NHR⁵, SO₃R⁵, NHSO₂R⁵, COR⁵, and NHCOR⁵;
wherein each (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, or (C₁-C₆)alkyl-heteroaryl is optionally substituted with one or more deuterium, ³H, ¹¹C, halogen, ¹⁸F, ⁷⁵Br, ⁷⁶Br, ¹²⁰I, ¹²³I, ¹²⁴I, ¹²⁵I, ¹³¹I, N(R⁵)₂, CN, OR⁵, SR⁵, SOR⁵, SO₂R⁵, SO₂NHR⁵, SO₃R⁵, NHSO₂R⁵, COR⁵, or NHCOR⁵; and

each occurrence of R⁵ is independently selected from the group consisting of hydrogen, deuterium, ³H, (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, and (C₁-C₆)alkyl-heteroaryl;
wherein each (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, and (C₁-C₆)alkyl-heteroaryl is optionally substituted with one or more deuterium, ³H, ¹¹C, ¹⁸F, ⁷⁵Br, ⁷⁶Br, ¹²⁰I, ¹²³I, ¹²⁴I, ¹²⁵I, or ¹³¹I;

wherein at least one of R, R¹, R², R³, R⁴, or R⁵ comprises at least one of ³H, ¹¹C, ¹⁸F, ⁷⁵Br, ⁷⁶Br, ¹²⁰I, ¹²³I, ¹²⁴I, ¹²⁵I, or ¹³¹I;

wherein when A is

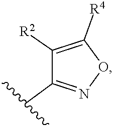

R is ¹¹CH₃, R¹ is hydrogen, and R² is phenyl, R⁴ is not CF₃;

wherein when A is

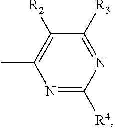

R is CH₃, R¹ and R² are hydrogen, and R³ is CF₃, R⁴ is not

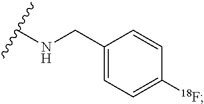

and

wherein when A is

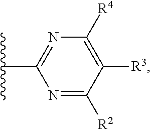

R is CH₃, R¹ and R³ are hydrogen, and R⁴ is O¹¹CH₃, R² is not

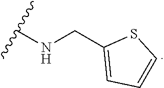 [SMILES: *NCc1cccs1]

In some embodiments, A is

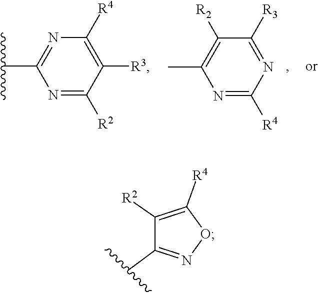

In some embodiments, A is

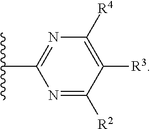

In some embodiments, A is

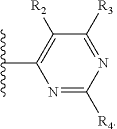

In some embodiments, A is

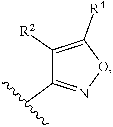

In some embodiments, R is selected from the group consisting of (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, (C₁-C₆)alkyl-heteroaryl, and N(R⁵)₂. In some embodiments, R is selected from the group consisting of (C₁-C₆)alkyl, (C₁-C₆)alkynyl, (C₁-C₆)heteroalkyl, (C₁-C₆)heteroalkynyl, (C₃-C₇)cycloalkyl, (C₃-C₇)heterocycloalkyl, (C₁-C₆)alkyl-(C₃-C₇)cycloalkyl, (C₁-C₆)alkyl-(C₃-C₇)heterocycloalkyl, aryl, (C₁-C₆)alkyl-aryl, heteroaryl, (C₁-C₆)alkyl-heteroaryl, and N(R⁵)₂. In some embodiments, R is (C₁-C₆)alkyl. In some embodiments, R is aryl. In some embodiments, R is heteroaryl. In some embodiments, R is (C₁-C₆)alkyl-aryl. In some embodiments, R is (C₁-C₆)alkyl-heteroaryl.

In some embodiments, each (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, or (C₁-C₆)alkyl-heteroaryl comprising R is optionally substituted with one or more deuterium, ³H, ¹¹C, halogen, ¹⁸F, ⁷⁵Br, ⁷⁶Br, ¹²⁰I, ¹²³I, ¹²⁴I, ¹²⁵I, ¹³¹I, N(R⁵)₂, CN, OR⁵, SR⁵, SOR⁵, SO₂R⁵, SO₂NHR⁵, SO₃R⁵, NHSO₂R⁵, COR⁵, or NHCOR⁵. In some embodiments, each (C₁-C₆)alkyl, (C₁-C₆)alkynyl, (C₁-C₆)heteroalkyl, (C₁-C₆)heteroalkynyl, (C₃-C₇)cycloalkyl, (C₃-C₇)heterocycloalkyl, (C₁-C₆)alkyl-(C₃-C₇)cycloalkyl, (C₁-C₆)alkyl-(C₃-C₇)heterocycloalkyl, aryl, (C₁-C₆)alkyl-aryl, heteroaryl, or (C₁-C₆)alkyl-heteroaryl comprising R is optionally substituted with one or more deuterium, ¹¹C, ¹⁸F, or ¹²³I.

In some embodiments, R is CH₃, ¹¹CH₃, CH₂¹⁸F, (CH₂)₂¹⁸F, CF₃, CF₂¹⁸F, CD₂¹⁸F,

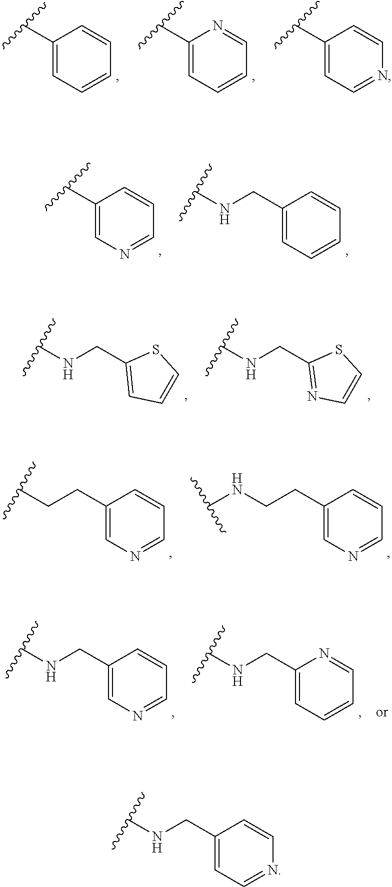

some embodiments, each

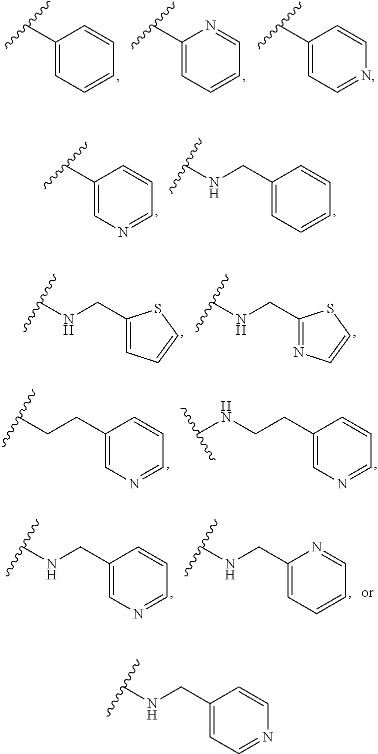

is optionally substituted with deuterium, ¹¹C, ¹⁸F, or ¹²³I. In some embodiments, R is CH₃, ¹¹CH₃, or (CH₂)₂¹⁸F. In some embodiments, R is CH₃. In some embodiments, R is ¹CH₃. In some embodiments, R is (CH₂)₂¹⁸F.

In some embodiments, R¹ is selected from the group consisting of hydrogen, deuterium, ³H, ¹¹C, halogen, ¹⁸F, ⁷⁵Br, ⁷⁶Br, ¹²⁰I, ¹²³I, ¹²⁴I, ¹²⁵I, ¹³¹I, (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, (C₁-C₆)alkyl-heteroaryl, N(R⁵)₂, CN, OR⁵, SR⁵, SOR⁵, SO₂R⁵, SO₂NHR⁵, SO₃R⁵, NHSO₂R⁵, COR⁵, and NHCOR⁵. In some embodiments, R¹ is selected from the group consisting of hydrogen, deuterium, halogen, ¹¹C, ¹⁸F, ¹²³I, (C₁-C₆)alkyl, (C₁-C₆)alkynyl, (C₁-C₆)heteroalkyl, (C₁-C₆)heteroalkynyl, (C₃-C₇)cycloalkyl, (C₃-C₇)heterocycloalkyl, (C₁-C₆)alkyl-(C₃-C₇)cycloalkyl, (C₁-C₆)alkyl-(C₃-C₇)heterocycloalkyl, aryl, (C₁-C₆)alkyl-aryl, heteroaryl, (C₁-C₆)alkyl-heteroaryl, and N(R⁵)₂. In some embodiments, R¹ is hydrogen, deuterium, ¹⁸F, ¹¹C, ¹²³I, (C₁-C₆)alkyl, aryl, heteroaryl, (C₁-C₆)alkyl-aryl, (C₁-C₆)alkyl-heteroaryl, or N(R⁵)₂. In some embodiments, R¹ is hydrogen. In some embodiments, R¹ is (C₁-C₆)alkyl. In some embodiments, R¹ is aryl. In some embodiments, R¹ is heteroaryl. In some embodiments, R¹ is (C₁-C₆)alkyl-aryl. In some embodiments, R¹ is (C₁-C₆)alkyl-heteroaryl. In some embodiments, R¹ is N(R⁵)₂. In some embodiments, R¹ is ¹⁸F.

In some embodiments, each (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, or (C₁-C₆)alkyl-heteroaryl of R¹ is optionally substituted with one or more deuterium, ³H, ¹¹C, halogen, ¹⁸F, ⁷⁵Br, ⁷⁶Br, ¹²⁰I, ¹²³I, ¹²⁴I, ¹²⁵I, ¹³¹I, N(R⁵)₂, CN, OR⁵, SR⁵, SOR⁵, SO₂R⁵, SO₂NHR⁵, SO₃R⁵, NHSO₂R⁵, COR⁵, or NHCOR⁵. In some embodiments, each (C₁-C₆)alkyl, aryl, heteroaryl, (C₁-C₆)alkyl-aryl, (C₁-C₆)alkyl-heteroaryl comprising R¹ is optionally substituted with one or more deuterium, ¹¹C, ¹⁸F, or ¹²³I.

In some embodiments, R¹ is hydrogen, deuterium, halogen, ¹⁸F, ¹²³I, CH₃, CH₂¹⁸F, (CH₂)₂¹⁸F, CF₃, CF₂¹⁸F, CD₂¹⁸F,

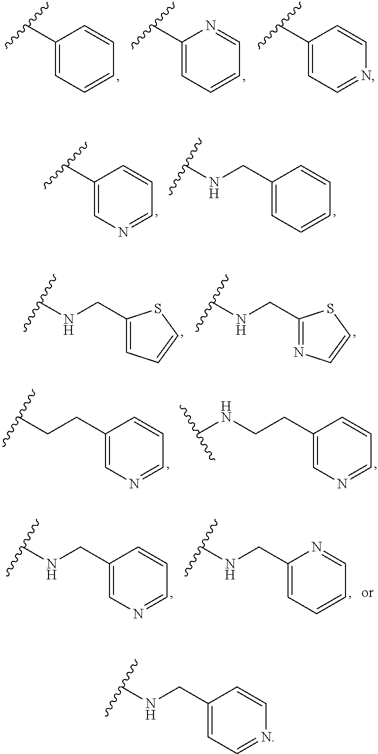

In some embodiments,

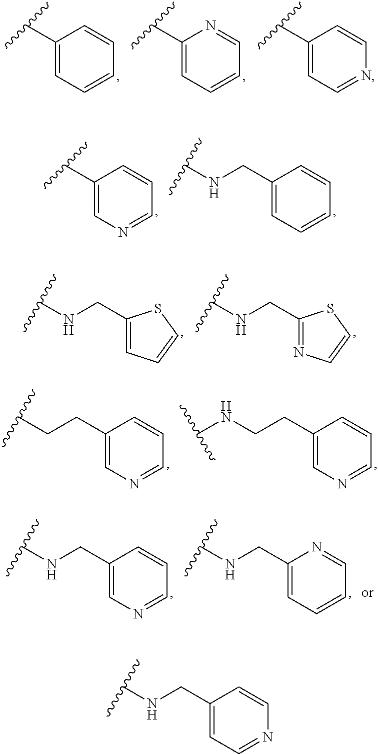

optionally substituted with deuterium, ¹¹C, ¹⁸F, or ¹²³I.

In some embodiments, R² is selected from the group consisting of hydrogen, deuterium, ³H, halogen, ¹¹C, ¹⁸F, ⁷⁵Br, ⁷⁶Br, ¹²⁰I, ¹²³I, ¹²⁴I, ¹²⁵I, ¹³¹I, (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, (C₁-C₆)alkyl-heteroaryl, N(R⁵)₂, CN, OR⁵, SR⁵, SOR⁵, SO₂R⁵, SO₂NHR⁵, SO₃R⁵, NHSO₂R⁵, COR⁵, and NHCOR⁵. In some embodiments, R² is selected from the group consisting of hydrogen, deuterium, halogen, ¹¹C, ¹⁸F, ¹²³I, (C₁-C₆)alkyl, (C₁-C₆)alkynyl, (C₁-C₆)heteroalkyl, (C₁-C₆)heteroalkynyl, (C₃-C₇)cycloalkyl, (C₃-C₇)heterocycloalkyl, (C₁-C₆)alkyl-(C₃-C₇)cycloalkyl, (C₁-C₆)alkyl-(C₃-C₇)heterocycloalkyl, aryl, (C₁-C₆)alkyl-aryl, heteroaryl, (C₁-C₆)alkyl-heteroaryl, and N(R⁵)₂. In some embodiments, R² is hydrogen, deuterium, ¹⁸F, ¹¹C, ¹⁸F, ¹²³I, (C₁-C₆)alkyl, aryl, heteroaryl, (C₁-C₆)alkyl-aryl, (C₁-C₆)alkyl-heteroaryl, or N(R⁵)₂. In some embodiments, R² is hydrogen. In some embodiments, R² is (C₁-C₆)alkyl. In some embodiments, R² is aryl. In some embodiments, R² is phenyl. In some embodiments, R² is heteroaryl. In some embodiments, R² is (C₁-C₆)alkyl-aryl. In some embodiments, R² is (C₁-C₆)alkyl-heteroaryl. In some embodiments, R² is N(R⁵)₂.

In some embodiments, each (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, or (C₁-C₆)alkyl-heteroaryl of R² is optionally substituted with one or more deuterium, ¹¹C, ³H, halogen, ¹⁸F, ⁷⁵Br, ⁷⁶Br, ¹²⁰I, ¹²³I, ¹²⁴I, ¹²⁵I, ¹³¹I, N(R⁵)₂, CN, OR⁵, SR⁵, SOR⁵, SO₂R⁵, SO₂NHR⁵, SO₃R⁵, NHSO₂R⁵, COR⁵, or NHCOR⁵. In some embodiments, each (C₁-C₆)alkyl, aryl, heteroaryl, (C₁-C₆)alkyl-aryl, or (C₁-C₆)alkyl-heteroaryl of R² is optionally substituted with one or more deuterium, ¹¹C, ¹⁸F, or ¹²³I.

In some embodiments, R² is hydrogen, deuterium, halogen, ¹⁸F, ¹²³I, CH₃, ¹¹CH₃, CH₂¹⁸F, (CH₂)₂¹⁸F, CF₃, CF₂¹⁸F, CD₂¹⁸F,

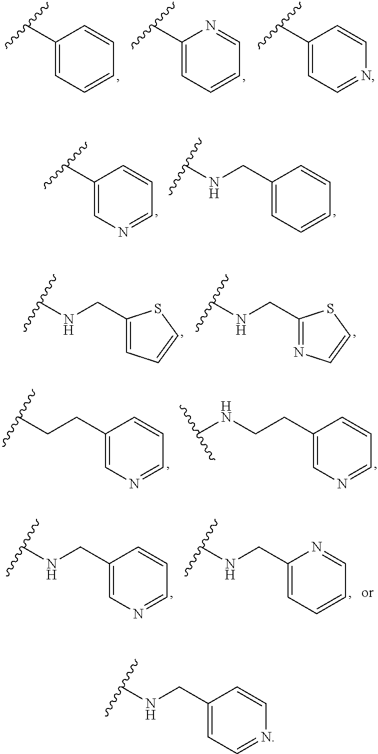

In some embodiments,

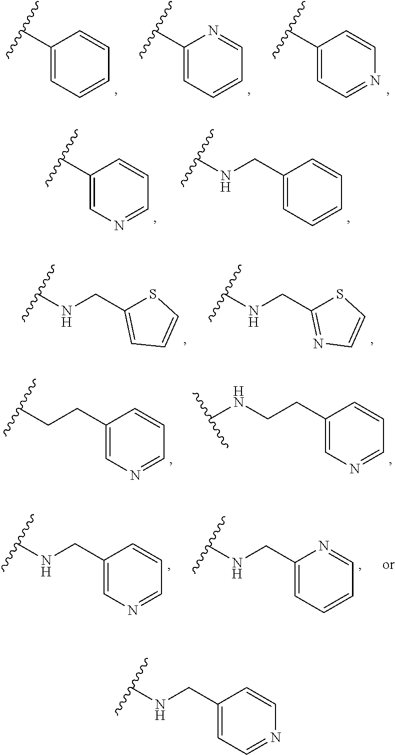

optionally substituted with deuterium, ¹¹C, ¹⁸F, or ¹²³I. In some embodiments, R² is hydrogen,

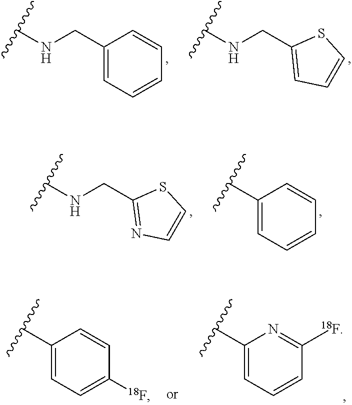

In some embodiments, R³ is selected from the group consisting of hydrogen, deuterium, ³H, ¹¹C, halogen, ¹⁸F, ⁷⁵Br, ⁷⁶Br, ¹²⁰I, ¹²³I, ¹²⁴I, ¹²⁵I, ¹³¹I, (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, (C₁-C₆)alkyl-heteroaryl, N(R⁵)₂, CN, OR⁵, SR⁵, SOR⁵, SO₂R⁵, SO₂NHR⁵, SO₃R⁵, NHSO₂R⁵, COR⁵, and NHCOR⁵. In some embodiments, R³ is selected from the group consisting of hydrogen, deuterium, halogen, ¹¹C, ¹⁸F, ¹²³I, (C₁-C₆)alkyl, (C₁-C₆)alkynyl, (C₁-C₆)heteroalkyl, (C₁-C₆)heteroalkynyl, (C₃-C₇)cycloalkyl, (C₃-C₇)heterocycloalkyl, (C₁-C₆)alkyl-(C₃-C₇)cycloalkyl, (C₁-C₆)alkyl-(C₃-C₇)heterocycloalkyl, aryl, (C₁-C₆)alkyl-aryl, heteroaryl, (C₁-C₆)alkyl-heteroaryl, and N(R⁵)₂. In some embodiments, R³ is hydrogen, deuterium, ¹¹C, ¹⁸F, ¹²³I, (C₁-C₆)alkyl, aryl, heteroaryl, (C₁-C₆)alkyl-aryl, (C₁-C₆)alkyl-heteroaryl, or N(R⁵)₂. In some embodiments, R³ is hydrogen. In some embodiments, R³ is (C₁-C₆)alkyl. In some embodiments, R³ is aryl. In some embodiments, R³ is heteroaryl. In some embodiments, R³ is (C₁-C₆)alkyl-aryl. In some embodiments, R³ is (C₁-C₆)alkyl-heteroaryl. In some embodiments, R³ is N(R⁵)₂.

In some embodiments, each (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, or (C₁-C₆)alkyl-heteroaryl of R³ is optionally substituted with one or more deuterium, ³H, ¹¹C, halogen, ¹⁸F, ⁷⁵Br, ⁷⁶Br, ¹²⁰I, ¹²³I, ¹²⁴I, ¹²⁵I, ¹³¹I, N(R⁵)₂, CN, OR⁵, SR⁵, SOR⁵, SO₂R⁵, SO₂NHR⁵, SO₃R⁵, NHSO₂R⁵, COR⁵, or NHCOR⁵. In some embodiments, each (C₁-C₆)alkyl, aryl, heteroaryl, (C₁-C₆)alkyl-aryl, or (C₁-C₆)alkyl-heteroaryl of R³ is optionally substituted with one or more deuterium, ¹¹C, ¹⁸F, or ¹²³I.

In some embodiments, R³ is hydrogen, deuterium, halogen, ¹⁸F, ¹²³I, CH₃, ¹¹CH₃, CH₂¹⁸F, (CH₂)₂¹⁸F, CF₃, CF₂¹⁸F, CD₂¹⁸F,

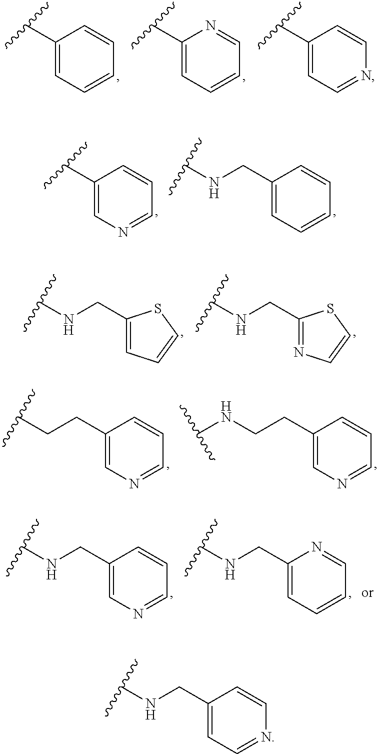

In some embodiments,

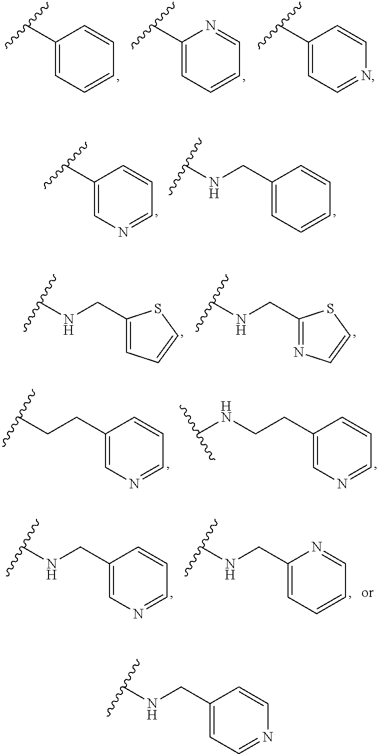

are optionally substituted with deuterium, ¹¹C, ¹⁸F, or ¹²³I. In some embodiments, R³ is hydrogen or ¹⁸F. In some embodiments, R³ is hydrogen. In some embodiments, R³ is ¹⁸F.

In some embodiments, R⁴ is selected from the group consisting of hydrogen, deuterium, ³H, ¹¹C, halogen, ¹⁸F, ⁷⁵Br, ⁷⁶Br, ¹²⁰I, ¹²³I, ¹²⁴I, ¹²⁵I, ¹³¹I, (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, (C₁-C₆)alkyl-heteroaryl, N(R⁵)₂, CN, OR⁵, SR⁵, SOR⁵, SO₂R⁵, SO₂NHR⁵, SO₃R⁵, NHSO₂R⁵, COR⁵, and NHCOR⁵. In some embodiments, R⁴ is selected from the group consisting of hydrogen, deuterium, halogen, ¹¹C, ¹⁸F, ¹²³I, (C₁-C₆)alkyl, (C₁-C₆)alkynyl, (C₁-C₆)heteroalkyl, (C₁-C₆)heteroalkynyl, (C₃-C₇)cycloalkyl, (C₃-C₇)heterocycloalkyl, (C₁-C₆)alkyl-(C₃-C₇)cycloalkyl, (C₁-C₆)alkyl-(C₃-C₇)heterocycloalkyl, aryl, (C₁-C₆)alkyl-aryl, heteroaryl, (C₁-C₆)alkyl-heteroaryl, and N(R⁵)₂. In some embodiments, R⁴ is hydrogen, deuterium, ¹¹C, ¹⁸F, ¹²³I, (C₁-C₆)alkyl, aryl, heteroaryl, (C₁-C₆)alkyl-aryl, (C₁-C₆)alkyl-heteroaryl, or N(R⁵)₂. In some embodiments, R⁴ is hydrogen. In some embodiments, R⁴ is (C₁-C₆)alkyl. In some embodiments, R⁴ is aryl. In some embodiments, R⁴ is heteroaryl. In some embodiments, R⁴ is (C₁-C₆)alkyl-aryl. In some embodiments, R⁴ is (C₁-C₆)alkyl-heteroaryl. In some embodiments, R⁴ is N(R⁵)₂.

In some embodiments, each (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, or (C₁-C₆)alkyl-heteroaryl of R⁴ is optionally substituted with one or more deuterium, ³H, ¹¹C, halogen, ¹⁸F, ⁷⁵Br, ⁷⁶Br, ¹²⁰I, ¹²³I, ¹²⁴I, ¹²⁵I, ¹³¹I, N(R⁵)₂, CN, OR⁵, SR⁵, SOR⁵, SO₂R⁵, SO₂NHR⁵, SO₃R⁵, NHSO₂R⁵, COR⁵, or NHCOR⁵. In some embodiments, each (C₁-C₆)alkyl, aryl, heteroaryl, (C₁-C₆)alkyl-aryl, or (C₁-C₆)alkyl-heteroaryl of R⁴ is optionally substituted with one or more deuterium, ¹¹C, ¹⁸F, or ¹²³I.

In some embodiments, R⁴ is hydrogen, deuterium, halogen, ¹⁸F, ¹²³I, CH₃, ¹¹CH₃, CH₂¹⁸F, (CH₂)₂¹⁸F, CF₃, CF₂¹⁸F, CD₂¹⁸F,

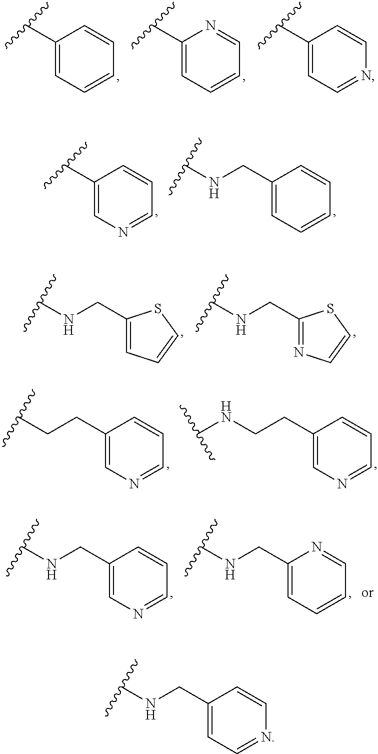

In some embodiments,

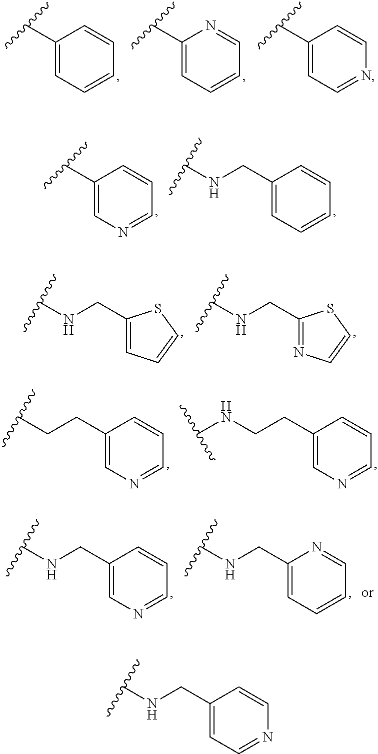

are optionally substituted with deuterium, ¹¹C, ¹⁸F, or ¹²³I. In some embodiments, R⁴ is halogen, ¹⁸F, CF₂¹⁸F, CF₃,

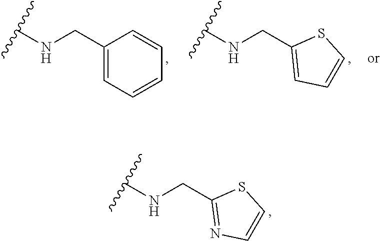

In some embodiments, R⁴ is CFs, CH₂¹⁸F, CD₂¹⁸F, or CF₂¹⁸F. In some embodiments, R⁴ is CFs. In some embodiments, R⁴ is CH₂¹⁸F. In some embodiments, R⁴ is CD₂¹⁸F. In some embodiments, R⁴ is CF₂¹⁸F.

In some embodiments, each occurrence of R⁵ is independently selected from the group consisting of hydrogen, deuterium, ³H, (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, and (C₁-C₆)alkyl-heteroaryl. In some embodiments, each occurrence of R⁵ is independently selected from the group consisting of hydrogen, deuterium, (C₁-C₆)alkyl, (C₁-C₆)alkynyl, (C₁-C₆)heteroalkyl, (C₁-C₆)heteroalkynyl, (C₃-C₇)cycloalkyl, (C₃-C₇)heterocycloalkyl, (C₁-C₆)alkyl-(C₃-C₇)cycloalkyl, (C₁-C₆)alkyl-(C₃-C₇)heterocycloalkyl, aryl, (C₁-C₆)alkyl-aryl, heteroaryl, and (C₁-C₆)alkyl-heteroaryl. In some embodiments, each occurrence of R⁵ is independently selected from the group consisting of hydrogen, (C₁-C₆)alkyl, aryl, heteroaryl, (C₁-C₆)alkyl-aryl, and (C₁-C₆)alkyl-heteroaryl. In some embodiments, R⁵ is hydrogen. In some embodiments, R⁵ is (C₁-C₆)alkyl. In some embodiments, R⁵ is aryl. In some embodiments, R⁵ is heteroaryl. In some embodiments, R⁵ is (C₁-C₆)alkyl-aryl.

In some embodiments, each (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, and (C₁-C₆)alkyl-heteroaryl of R⁵ is optionally substituted with one or more deuterium, ³H, ¹¹C, ¹⁸F, ⁷⁵Br, ⁷⁶Br, ¹²⁰I, ¹²³I, ¹²⁴I, ¹²⁵I, or ¹³¹I. In some embodiments, each (C₁-C₆)alkyl, aryl, heteroaryl, (C₁-C₆)alkyl-aryl, and (C₁-C₆)alkyl-heteroaryl of R⁵ is optionally substituted with one or more deuterium, ¹¹C, ¹⁸F, or ¹²³I.

In some embodiments, each occurrence of R⁵ is independently hydrogen,

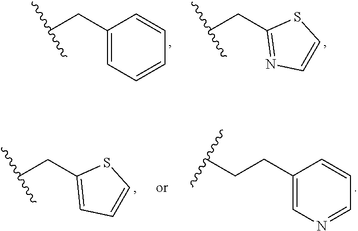

In some embodiments, each occurrence of R⁵ is independently hydrogen,

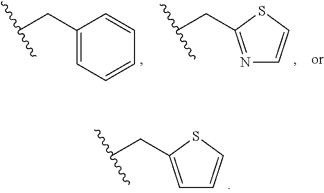

In some embodiments, A is

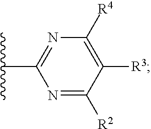

R is CH₃ or ¹¹CH₃; R¹ and R³ are hydrogen; R² is N(R⁵)₂; R⁴ is F, Cl, CFs, or ¹⁸F; and each occurrence of R⁵ is independently hydrogen, (C₁-C₆)alkyl-aryl, or (C₁-C₆)alkyl-heteroaryl. In some embodiments, each occurrence of R⁵ is independently hydrogen,

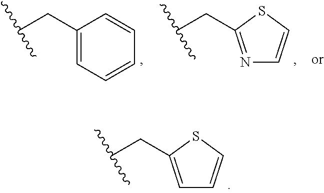

In some embodiments, R² is

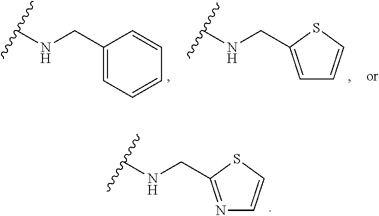

In some embodiments, A is

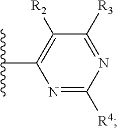

R is ¹¹CH₃; R¹ and R² are hydrogen; R² is ¹⁸F; R⁴ is (NR⁵)₂; and each occurrence of R⁵ is independently hydrogen, (C₁-C₆)alkyl-aryl, or (C₁-C₆)alkyl-heteroaryl. In some embodiments, each occurrence of R⁵ is independently hydrogen,

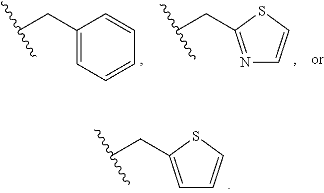

In some embodiments, R² is

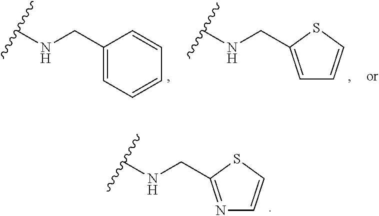

In some embodiments, A is

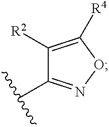

R and R⁴ are (C₁-C₆)alkyl, optionally substituted with one or more halogen, deuterium, or ¹⁸F; R¹ is hydrogen or ¹⁸F; and R² is aryl, optionally substituted with ¹⁸F, or heteroaryl, optionally substituted with ¹⁸F. In some embodiments, R is CH₃ or (CH₂)₂¹⁸F. In some embodiments, R² is

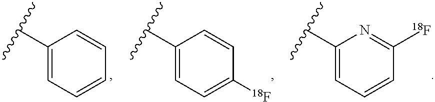

In some embodiments, R⁴ is CF₃, CH₂¹⁸F, CD₂¹⁸F, or CF₂¹⁸F.

In some embodiments, the compound of Formula I is

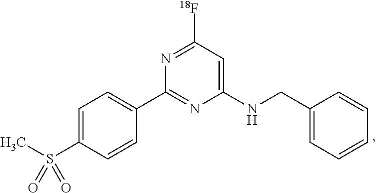 [SMILES: CS(=O)(=O)c1ccc(-c2nc([18F])cc(NCc3ccccc3)n2)cc1]

or a pharmaceutically acceptable salt thereof. In some embodiments, the compound of Formula I is

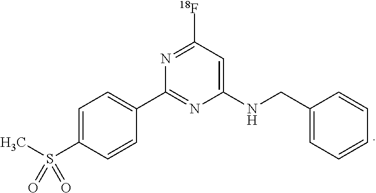 [SMILES: CS(=O)(=O)c1ccc(-c2nc([18F])cc(NCc3ccccc3)n2)cc1]

In some embodiments, the compound of Formula I is

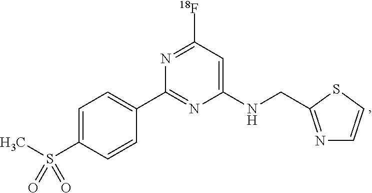 [SMILES: CS(=O)(=O)c1ccc(-c2nc([18F])cc(NCc3nccs3)n2)cc1]

or a pharmaceutically acceptable salt thereof. In some embodiments, the compound of Formula I is

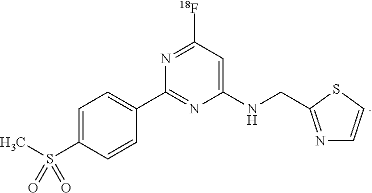 [SMILES: CS(=O)(=O)c1ccc(-c2nc([18F])cc(NCc3nccs3)n2)cc1]

In some embodiments, the compound of Formula I is

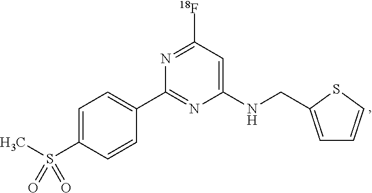 [SMILES: CS(=O)(=O)c1ccc(-c2nc([18F])cc(NCc3cccs3)n2)cc1]

or a pharmaceutically acceptable salt thereof. In some embodiments, the compound of Formula I is

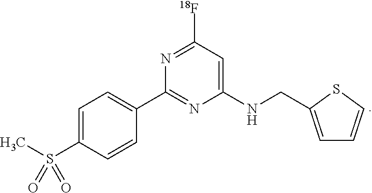 [SMILES: CS(=O)(=O)c1ccc(-c2nc([18F])cc(NCc3cccs3)n2)cc1]

In some embodiments, the compound of Formula I is

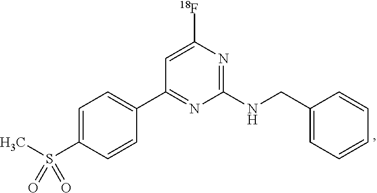 [SMILES: CS(=O)(=O)c1ccc(-c2cc([18F])nc(NCc3ccccc3)n2)cc1]

or a pharmaceutically acceptable salt thereof. In some embodiments, the compound of Formula I is

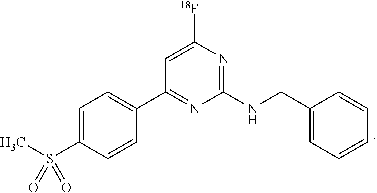 [SMILES: CS(=O)(=O)c1ccc(-c2cc([18F])nc(NCc3ccccc3)n2)cc1]

In some embodiments, the compound of Formula I is

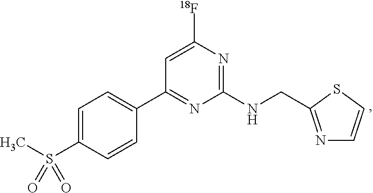 [SMILES: CS(=O)(=O)c1ccc(-c2cc([18F])nc(NCc3nccs3)n2)cc1]

or a pharmaceutically acceptable salt thereof. In some embodiments, the compound of Formula I is

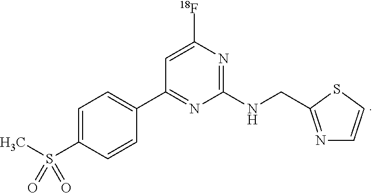 [SMILES: CS(=O)(=O)c1ccc(-c2cc([18F])nc(NCc3nccs3)n2)cc1]

In some embodiments, the compound of Formula I is

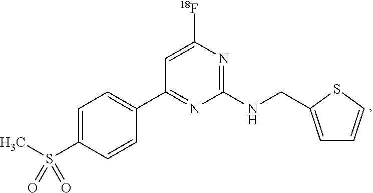 [SMILES: CS(=O)(=O)c1ccc(-c2cc([18F])nc(NCc3cccs3)n2)cc1]

or a pharmaceutically acceptable salt thereof. In some embodiments, the compound of Formula I is

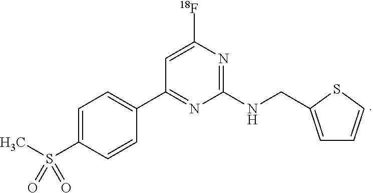 [SMILES: CS(=O)(=O)c1ccc(-c2cc([18F])nc(NCc3cccs3)n2)cc1]

In some embodiments, the compound of Formula I is

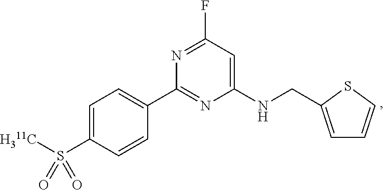 [SMILES: [11CH3]S(=O)(=O)c1ccc(-c2nc(F)cc(NCc3cccs3)n2)cc1]

or a pharmaceutically acceptable salt thereof. In some embodiments, the compound of Formula I is

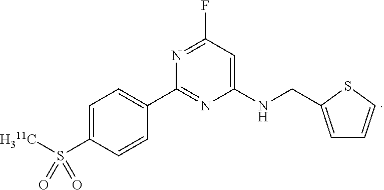 [SMILES: [11CH3]S(=O)(=O)c1ccc(-c2nc(F)cc(NCc3cccs3)n2)cc1]

In some embodiments, the compound of Formula I is

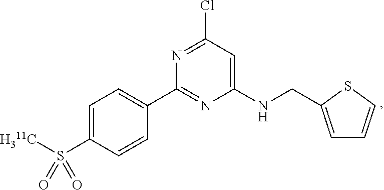 [SMILES: [11CH3]S(=O)(=O)c1ccc(-c2nc(Cl)cc(NCc3cccs3)n2)cc1]

or a pharmaceutically acceptable salt thereof. In some embodiments, the compound of Formula I is

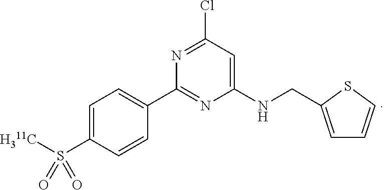 [SMILES: [11CH3]S(=O)(=O)c1ccc(-c2nc(Cl)cc(NCc3cccs3)n2)cc1]

In some embodiments, the compound of Formula I is

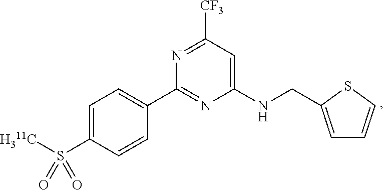 [SMILES: [11CH3]S(=O)(=O)c1ccc(-c2nc(NCc3cccs3)cc(C(F)(F)F)n2)cc1]

or a pharmaceutically acceptable salt thereof. In some embodiments, the compound of Formula I is

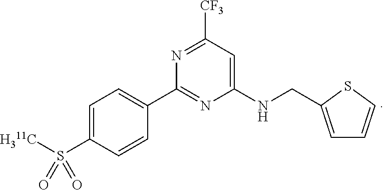 [SMILES: [11CH3]S(=O)(=O)c1ccc(-c2nc(NCc3cccs3)cc(C(F)(F)F)n2)cc1]

In some embodiments, the compound of Formula I is

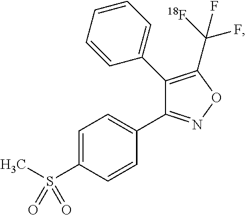 [SMILES: CS(=O)(=O)c1ccc(-c2noc(C(F)(F)[18F])c2-c2ccccc2)cc1]

or a pharmaceutically acceptable salt thereof. In some embodiments, the compound of Formula I is

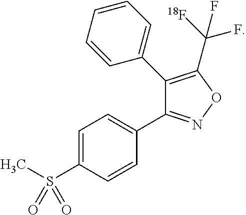 [SMILES: CS(=O)(=O)c1ccc(-c2noc(C(F)(F)[18F])c2-c2ccccc2)cc1]

In some embodiments, the compound of Formula I is

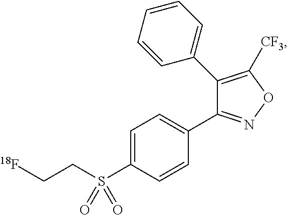 [SMILES: O=S(=O)(CC[18F])c1ccc(-c2noc(C(F)(F)F)c2-c2ccccc2)cc1]

or a pharmaceutically acceptable salt thereof. In some embodiments, the compound of Formula I is

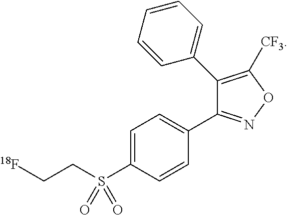 [SMILES: O=S(=O)(CC[18F])c1ccc(-c2noc(C(F)(F)F)c2-c2ccccc2)cc1]

In some embodiments, the compound of Formula I is

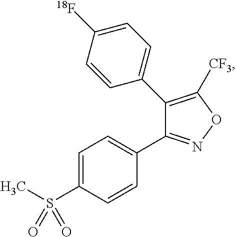 [SMILES: CS(=O)(=O)c1ccc(-c2noc(C(F)(F)F)c2-c2ccc([18F])cc2)cc1]

or a pharmaceutically acceptable salt thereof. In some embodiments, the compound of Formula I is

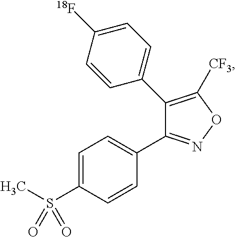 [SMILES: CS(=O)(=O)c1ccc(-c2noc(C(F)(F)F)c2-c2ccc([18F])cc2)cc1]

In some embodiments, the compound of Formula I is

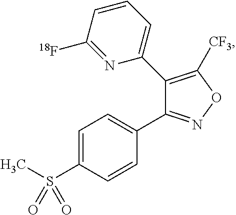 [SMILES: CS(=O)(=O)c1ccc(-c2noc(C(F)(F)F)c2-c2cccc([18F])n2)cc1]

or a pharmaceutically acceptable salt thereof. In some embodiments, the compound of Formula I is

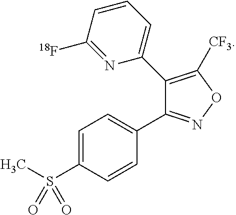 [SMILES: CS(=O)(=O)c1ccc(-c2noc(C(F)(F)F)c2-c2cccc([18F])n2)cc1]

In some embodiments, the compound of Formula I is

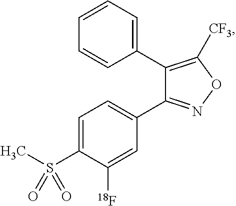 [SMILES: CS(=O)(=O)c1ccc(-c2noc(C(F)(F)F)c2-c2ccccc2)cc1[18F]]

or a pharmaceutically acceptable salt thereof. In some embodiments, the compound of Formula I is

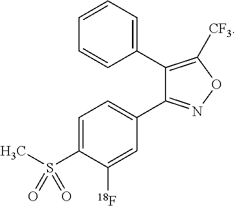 [SMILES: CS(=O)(=O)c1ccc(-c2noc(C(F)(F)F)c2-c2ccccc2)cc1[18F]]

In some embodiments, the compound of Formula I is

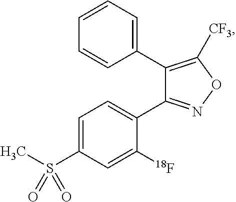 [SMILES: CS(=O)(=O)c1ccc(-c2noc(C(F)(F)F)c2-c2ccccc2)c([18F])c1]

or a pharmaceutically acceptable salt thereof. In some embodiments, the compound of Formula I is

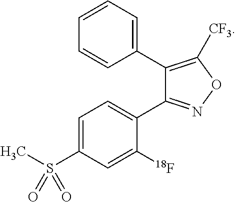 [SMILES: CS(=O)(=O)c1ccc(-c2noc(C(F)(F)F)c2-c2ccccc2)c([18F])c1]

In some embodiments, the compound of Formula I is

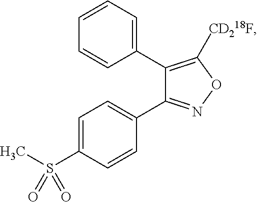

or a pharmaceutically acceptable salt thereof. In some embodiments, the compound of Formula I is

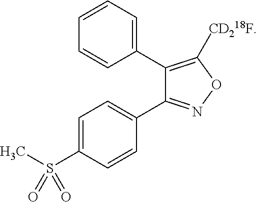

In some embodiments, the compound of Formula I is

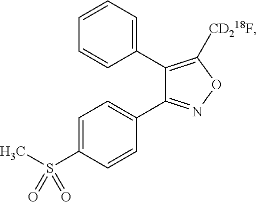

or a pharmaceutically acceptable salt thereof. In some embodiments, the compound of Formula I is

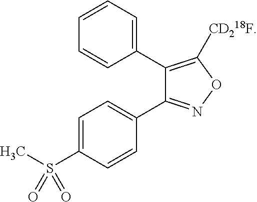

In some embodiments, the specific activity of the ¹⁸F-labelled compounds of Formula I is about 1 to about 5 Ci/μmol. In some embodiments, the molar activity of the ¹⁸F-labelled compounds of Formula I is about 0.5 Ci/μmol or greater. In some embodiments, the molar activity of the ¹⁸F-labelled compounds of Formula I is about 0.5 to about 2.5 Ci/μmol.

In some embodiments, the compound of Formula I can exist as a salt form. In some embodiments, the salt form is pharmaceutically acceptable. Non-limiting examples of salts, including pharmaceutically acceptable salts, are those derived from inorganic or organic acids (e.g., hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, and naphthalene-2 sulfonic acids); salts derived from inorganic or organic bases (e.g., sodium, potassium, calcium, magnesium, zinc, ammonia, lysine, arginine, histidine, polyhydroxylated amines, and tetrafluoroborates); and hemi-salts, such as those derived from acids comprising two carboxylic acid groups (e.g., malic acid, fumaric acid, maleic acid, succinic acid, tartaric acid, glutaric acid, oxalic acid, adipic acid, and citric acid) or diprotic mineral acids (e.g., sulfuric acid). Other exemplary salts are found, for example, in Berge, et al. (J. Pharm. Sci. 1977, 66(1): 1 (incorporated by reference herein in its entirety).

Processes for Compound Synthesis

In yet another aspect, a process for synthesizing a compound of Formula I

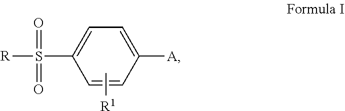

or a pharmaceutically acceptable salt thereof, comprises reacting one or more halogen atom(s) or thioester(s) in the compound with one or more radionuclide source(s) independently selected from the group consisting of deuterium, ³H, ¹¹C, ¹⁸F, ⁷⁵Br, ⁷⁶Br, ¹²⁰I, ¹²³I, ¹²⁴I, ¹²⁵I, and ¹³¹I.

In some embodiments, the one or more halogen atom(s) are independently chlorine or bromine. In some embodiments, the one or more halogen atom(s) are chlorine. In some embodiments, the one or more halogen atom(s) are bromine.

In some embodiments, the one or more thioester(s) comprise

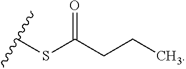 [SMILES: *SC(=O)CCC]

In some embodiments, the radionuclide source is a ¹⁸F source. In some embodiments, the a ¹⁸F source comprises a ¹⁸F⁻ source. Non-limiting examples of ¹⁸F⁻ sources include K¹⁸F and a tetraalkylammonium [¹⁸F]fluoride (e.g., tetrabutylammonium [¹⁸F]fluoride and tetraethylammonium [¹⁸F]fluoride). In some embodiments, the ¹⁸F source is K¹⁸F. In some embodiments, the ¹⁸F source comprises a [¹⁸F]fluoroalkyl tosylate. A non-limiting example of a [¹⁸F]fluoroalkyl tosylate is 2-[¹⁸F]fluoroethyl tosylate.

In some embodiments, the process further comprises a chelating agent. In some embodiments, the chelating agent is capable of chelating a cationic metal, such as potassium ion, sodium ion, calcium ion, lithium ion, and the like. In some embodiments, the chelating agent is capable of chelating potassium ion. Non-limiting examples of potassium chelating agents include crown ethers (e.g., 12-crown-4, 15-crown-5, 18-crown-6, dibenzo-18-crown-6, and diaza-18-crown-6) and cryptands. In some embodiments, the cryptand is a [2.2.2]cryptand. In some embodiments, the cryptand is Kryptofix® 222 (“K₂₂₂”).

In some embodiments, the process further comprises a base. Non-limiting examples of bases include organic bases, such as tetrabutylammonium hydroxide, pyrrolidine, lithium diisopropylamide, 2,6-lutidine, lithium diethylamide, sodium methoxide, n-butyllithium, lithium hexamethyldisilazide, sodium hexamethyldisilazide, potassium hexamethyl disilazide, potassium tert-butoxide, piperidine, piperazine, sodium acetate, morpholine, diethylamine, tetramethylpiperidine, diisopropylamine, and triethylamine; and inorganic bases, such as sodium hydroxide, potassium hydroxide, calcium hydroxide, potassium carbonate, sodium carbonate, cesium carbonate, potassium bicarbonate, sodium hydride, and potassium hydride. In some embodiments, the base is a carbonate. In some embodiments, the base is potassium carbonate. In some embodiments, the base is tetrabutylammonium hydroxide. In some embodiments, the base is pyrrolidine. In some embodiments, the base is 2,6-lutidine.

In some embodiments, the process further comprises a polar aprotic solvent. Non-limiting examples of polar aprotic solvents include A. A-dimethylformamide (“DMF”), hexamethylphosphoramide (“HMPA”), dimethyl sulfoxide (“DMSO”), tetramethylene sulfone, 1,4-dioxane, acetone, ether (e.g., diethyl ether, diphenyl ether, and tetrahydrofuran (“THF”)) acetonitrile, methyl ethyl ketone, ethyl acetate, and combinations thereof. In some embodiments, the polar aprotic solvent is THF, DMF, DMSO, or combinations thereof. In some embodiments, the polar aprotic solvent is THF. In some embodiments, the polar aprotic solvent is DMF. In some embodiments, the polar aprotic solvent is DMSO. In some embodiments, the solvent is anhydrous (i.e., comprises less than about 1% water).

In some embodiments, the radionuclide source is a ¹¹C source. In some embodiments, the ¹¹C source comprises a [¹¹C]alkyl halide and a [¹¹C]alkyl triflate. A non-limiting example of a [¹¹C]alkyl halide is ¹¹CH₃I. A non-limiting example of a [¹¹C]alkyl triflate is ¹¹CH₃OTf. In some embodiments, the ¹¹C source is ¹¹CH₃I. In some embodiments, the ¹¹C source is ¹¹CH₃OTf.

In some embodiments, the process further comprises an oxidant. In some embodiments, the process further comprises an oxidant when the radionuclide source is a ¹¹C source. Non-limiting examples of oxidants include Oxone®, optionally with a water/THF solvent, a peroxyacid (e.g., meta-chloroperoxybenzoic acid), potassium peroxymonosulfate, H₂O₂, NaIO₄, t-BuOCl, Ca(OCl)₂, NaClO₂, NaOCl, a dioxirane, and KMnO₄. In some embodiments, the oxidant is Oxone® or potassium peroxymonosulfate. In some embodiments, the oxidant is Oxone®.

In some embodiments, the process comprises reacting one or more chlorine or bromine atom(s) in a compound of Formula I with a ¹⁸F source. In some embodiments, the ¹⁸F source is K¹⁸F. In some embodiments, the ¹⁸F source is 2-[¹⁸F]fluoroethyl tosylate.

In some embodiments, the process comprises reacting one or more thioester(s) in a compound of Formula I with a ¹¹C source in the presence of an oxidant. In some embodiments, the ¹¹C source is ¹¹CH₃I. In some embodiments, the ¹¹C source is ¹¹CH₃OTf. In some embodiments, the oxidant is Oxone®.

In some embodiments, the compound of Formula I is

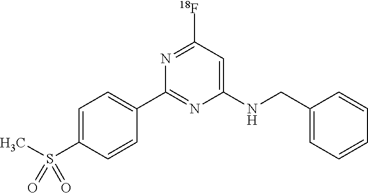 [SMILES: CS(=O)(=O)c1ccc(-c2nc([18F])cc(NCc3ccccc3)n2)cc1]

or a pharmaceutically acceptable salt thereof; and the process comprises reacting the compound

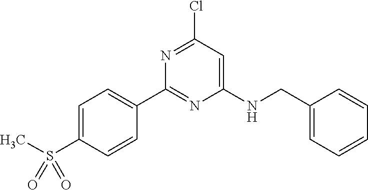 [SMILES: CS(=O)(=O)c1ccc(-c2nc(Cl)cc(NCc3ccccc3)n2)cc1]

with a ¹⁸F source. In some embodiments, the ¹⁸F source is a ¹⁸F⁻ source. In some embodiments, the compound of Formula I is

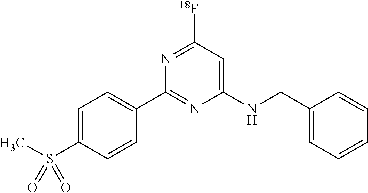 [SMILES: CS(=O)(=O)c1ccc(-c2nc([18F])cc(NCc3ccccc3)n2)cc1]

and the process comprises reacting the compound

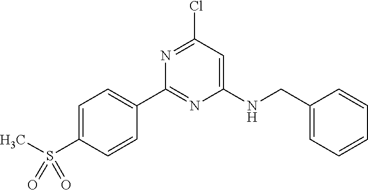 [SMILES: CS(=O)(=O)c1ccc(-c2nc(Cl)cc(NCc3ccccc3)n2)cc1]

with a ¹⁸F source. In some embodiments, the ¹⁸F source is a ¹⁸F⁻ source.

In some embodiments, the compound of Formula I is

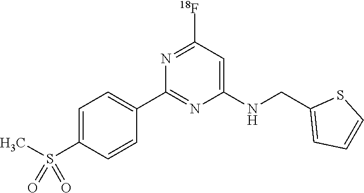 [SMILES: CS(=O)(=O)c1ccc(-c2nc([18F])cc(NCc3cccs3)n2)cc1]

or a pharmaceutically acceptable salt thereof; and the process comprises reacting the compound

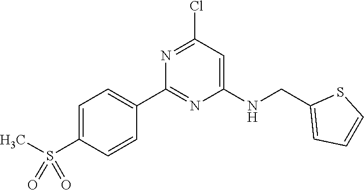 [SMILES: CS(=O)(=O)c1ccc(-c2nc(Cl)cc(NCc3cccs3)n2)cc1]

with a ¹⁸F source. In some embodiments, the ¹⁸F source is a ¹⁸F⁻ source. In some embodiments, the compound of Formula I is

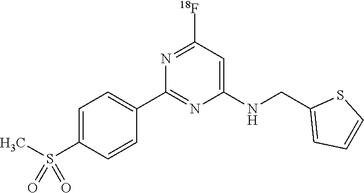 [SMILES: CS(=O)(=O)c1ccc(-c2nc([18F])cc(NCc3cccs3)n2)cc1]

and the process comprises reacting the compound

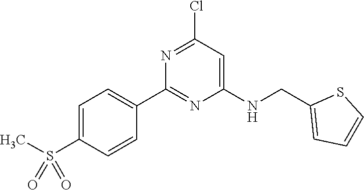 [SMILES: CS(=O)(=O)c1ccc(-c2nc(Cl)cc(NCc3cccs3)n2)cc1]

with a ¹⁸F source. In some embodiments, the ¹⁸F source is a ¹⁸F⁻ source.

In some embodiments, the compound of Formula I is

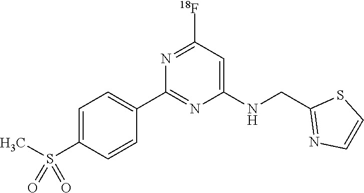 [SMILES: CS(=O)(=O)c1ccc(-c2nc([18F])cc(NCc3nccs3)n2)cc1]

a pharmaceutically acceptable salt thereof; and the process comprises reacting the compound

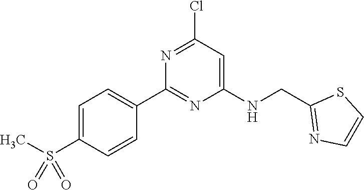 [SMILES: CS(=O)(=O)c1ccc(-c2nc(Cl)cc(NCc3nccs3)n2)cc1]

with a ¹⁸F source. In some embodiments, the ¹⁸F source is a ¹⁸F⁻ source. In some embodiments, the compound of Formula I is

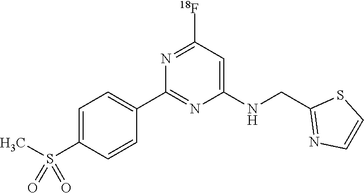 [SMILES: CS(=O)(=O)c1ccc(-c2nc([18F])cc(NCc3nccs3)n2)cc1]

and the process comprises reacting the compound

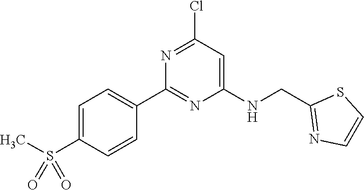 [SMILES: CS(=O)(=O)c1ccc(-c2nc(Cl)cc(NCc3nccs3)n2)cc1]

with a ¹⁸F source. In some embodiments, the ¹⁸F source is a ¹⁸F⁻ source.

In some embodiments, the compound of Formula I is

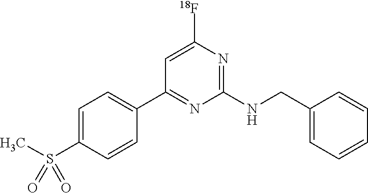 [SMILES: CS(=O)(=O)c1ccc(-c2cc([18F])nc(NCc3ccccc3)n2)cc1]

or a pharmaceutically acceptable salt thereof; and the process comprises reacting the compound

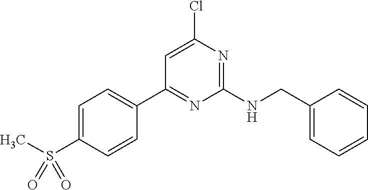 [SMILES: CS(=O)(=O)c1ccc(-c2cc(Cl)nc(NCc3ccccc3)n2)cc1]

with a ¹⁸F source. In some embodiments, the ¹⁸F source is a ¹⁸F⁻ source. In some embodiments, the compound of Formula I is

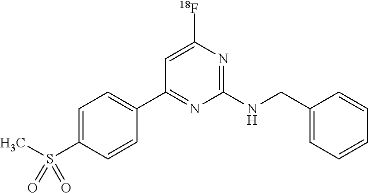 [SMILES: CS(=O)(=O)c1ccc(-c2cc([18F])nc(NCc3ccccc3)n2)cc1]

and the process comprises reacting the compound

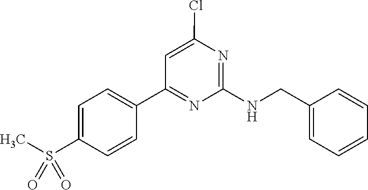 [SMILES: CS(=O)(=O)c1ccc(-c2cc(Cl)nc(NCc3ccccc3)n2)cc1]

with a ¹⁸F source. In some embodiments, the ¹⁸F source is a ¹⁸F⁻ source.

In some embodiments, the compound of Formula I is

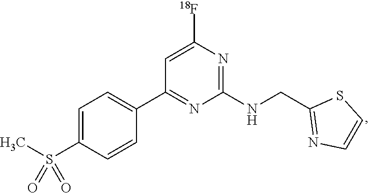 [SMILES: CS(=O)(=O)c1ccc(-c2cc([18F])nc(NCc3nccs3)n2)cc1]

or a pharmaceutically acceptable salt thereof; and the process comprises reacting the compound

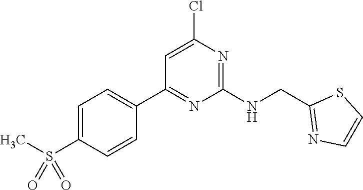 [SMILES: CS(=O)(=O)c1ccc(-c2cc(Cl)nc(NCc3nccs3)n2)cc1]

with a ¹⁸F source. In some embodiments, the ¹⁸F source is a ¹⁸F⁻ source. In some embodiments, the compound of Formula I is

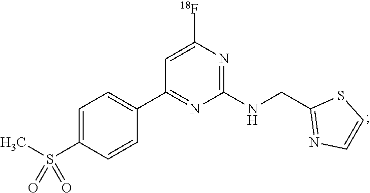 [SMILES: CS(=O)(=O)c1ccc(-c2cc([18F])nc(NCc3nccs3)n2)cc1]

and the process comprises reacting the compound

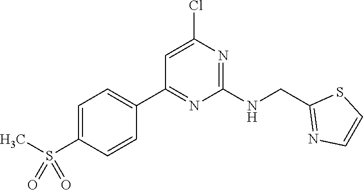 [SMILES: CS(=O)(=O)c1ccc(-c2cc(Cl)nc(NCc3nccs3)n2)cc1]

with a ¹⁸F source. In some embodiments, the ¹⁸F source is a ¹⁸F⁻ source.

In some embodiments, the compound of Formula I is

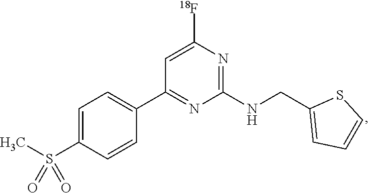 [SMILES: CS(=O)(=O)c1ccc(-c2cc([18F])nc(NCc3cccs3)n2)cc1]

or a pharmaceutically acceptable salt thereof; and the process comprises reacting the compound

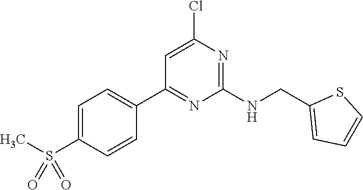 [SMILES: CS(=O)(=O)c1ccc(-c2cc(Cl)nc(NCc3cccs3)n2)cc1]

with a ¹⁸F source. In some embodiments, the ¹⁸F source is a ¹⁸F⁻ source. In some embodiments, the compound of Formula I is

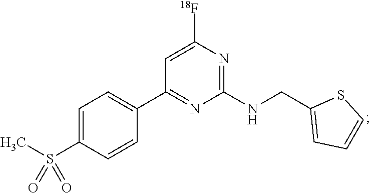 [SMILES: CS(=O)(=O)c1ccc(-c2cc([18F])nc(NCc3cccs3)n2)cc1]

and the process comprises reacting the compound

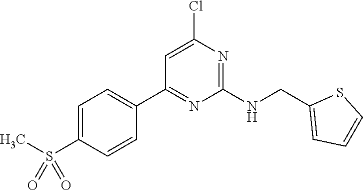 [SMILES: CS(=O)(=O)c1ccc(-c2cc(Cl)nc(NCc3cccs3)n2)cc1]

with a ¹⁸F source. In some embodiments, the ¹⁸F source is a ¹⁸F⁻ source.

In some embodiments, the compound of Formula I is

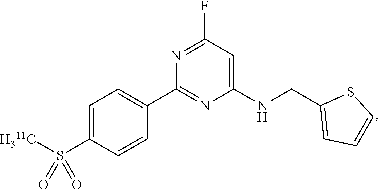 [SMILES: [11CH3]S(=O)(=O)c1ccc(-c2nc(F)cc(NCc3cccs3)n2)cc1]

or a pharmaceutically acceptable salt thereof; and the process comprises reacting the compound

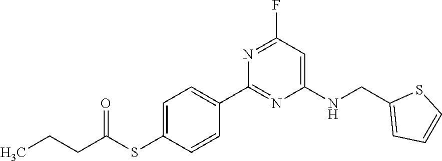 [SMILES: CCCC(=O)Sc1ccc(-c2nc(F)cc(NCc3cccs3)n2)cc1]

with a ¹¹C source and an oxidant. In some embodiments, the ¹¹C source is ¹¹CH₃I and/or ¹¹CH₃OTf and the oxidant is Oxone®. In some embodiments, the compound of Formula I is

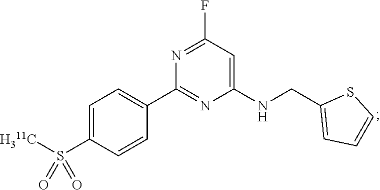 [SMILES: [11CH3]S(=O)(=O)c1ccc(-c2nc(F)cc(NCc3cccs3)n2)cc1]

and the process comprises reacting the compound

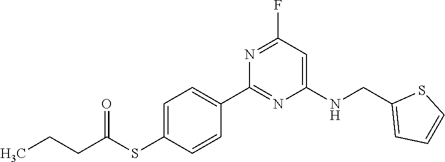 [SMILES: CCCC(=O)Sc1ccc(-c2nc(F)cc(NCc3cccs3)n2)cc1]

with a ¹¹C source and an oxidant. In some embodiments, the ¹¹C source is ¹¹CH₃I and/or ¹¹CH₃OTf and the oxidant is Oxone®.

In some embodiments, the compound of Formula I is

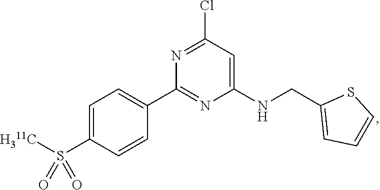 [SMILES: [11CH3]S(=O)(=O)c1ccc(-c2nc(Cl)cc(NCc3cccs3)n2)cc1]

or a pharmaceutically acceptable salt thereof; and the process comprises reacting the compound

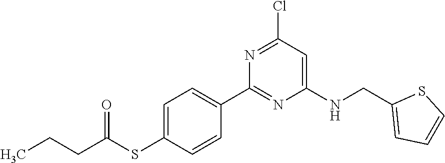 [SMILES: CCCC(=O)Sc1ccc(-c2nc(Cl)cc(NCc3cccs3)n2)cc1]

with a ¹¹C source and an oxidant. In some embodiments, the ¹¹C source is ¹¹CH₃I and/or ¹¹CH₃OTf and the oxidant is Oxone®. In some embodiments, the compound of Formula I is

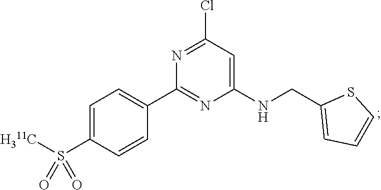 [SMILES: [11CH3]S(=O)(=O)c1ccc(-c2nc(Cl)cc(NCc3cccs3)n2)cc1]

and the process comprises reacting the compound

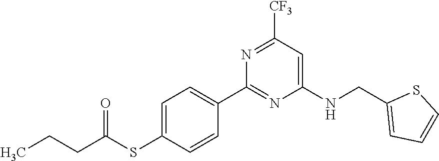 [SMILES: CCCC(=O)Sc1ccc(-c2nc(NCc3cccs3)cc(C(F)(F)F)n2)cc1]

with a ¹¹C source and an oxidant. In some embodiments, the ¹¹C source is ¹¹CH₃I and/or ¹¹CH₃OTf and the oxidant is Oxone®.

In some embodiments, the compound of Formula I is

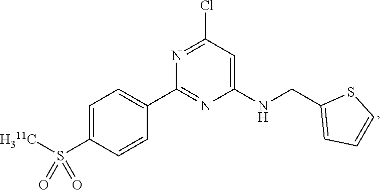 [SMILES: [11CH3]S(=O)(=O)c1ccc(-c2nc(Cl)cc(NCc3cccs3)n2)cc1]

or a pharmaceutically acceptable salt thereof; and the process comprises reacting the compound

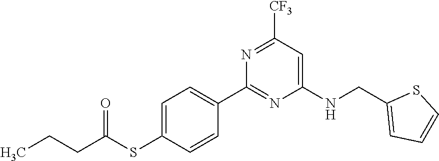 [SMILES: CCCC(=O)Sc1ccc(-c2nc(NCc3cccs3)cc(C(F)(F)F)n2)cc1]

with a ¹¹C source and an oxidant. In some embodiments, the ¹¹C source is ¹¹CH₃I and/or ¹¹CH₃OTf and the oxidant is Oxone®. In some embodiments, the compound of Formula I is

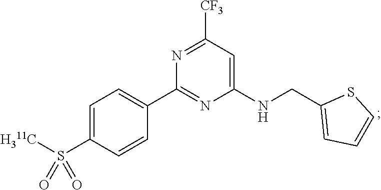 [SMILES: [11CH3]S(=O)(=O)c1ccc(-c2nc(NCc3cccs3)cc(C(F)(F)F)n2)cc1]

and the process comprises reacting the compound

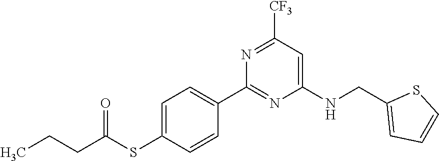 [SMILES: CCCC(=O)Sc1ccc(-c2nc(NCc3cccs3)cc(C(F)(F)F)n2)cc1]

with a ¹¹C source and an oxidant. In some embodiments, the ¹¹C source is ¹¹CH₃I and/or ¹¹CH₃OTf and the oxidant is Oxone®.

In some embodiments, the compound of Formula I is

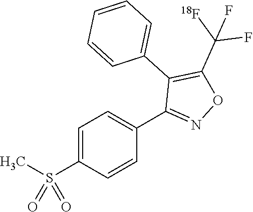 [SMILES: CS(=O)(=O)c1ccc(-c2noc(C(F)(F)[18F])c2-c2ccccc2)cc1]

or a pharmaceutically acceptable salt thereof; and the process comprises reacting the compound

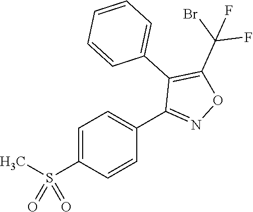 [SMILES: CS(=O)(=O)c1ccc(-c2noc(C(F)(F)Br)c2-c2ccccc2)cc1]

with a ¹⁸F source. In some embodiments, the ¹⁸F source is a ¹⁸F⁻ source. In some embodiments, the compound of Formula I is

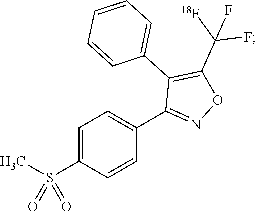 [SMILES: CS(=O)(=O)c1ccc(-c2noc(C(F)(F)[18F])c2-c2ccccc2)cc1]

and the process comprises reacting the compound

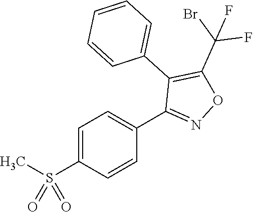 [SMILES: CS(=O)(=O)c1ccc(-c2noc(C(F)(F)Br)c2-c2ccccc2)cc1]

with a ¹⁸F source. In some embodiments, the ¹⁸F source is a ¹⁸F⁻ source.

In some embodiments, the compound of Formula I is

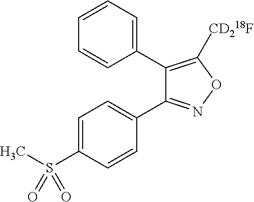

or a pharmaceutically acceptable salt thereof; and the process comprises reacting the compound

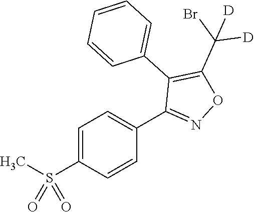 [SMILES: [2H]C([2H])(Br)c1onc(-c2ccc(S(C)(=O)=O)cc2)c1-c1ccccc1]

with a ¹⁸F source. In some embodiments, the ¹⁸F source is a ¹⁸F⁻ source. In some embodiments, the compound of Formula I is

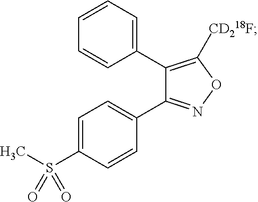

and the process comprises reacting the compound

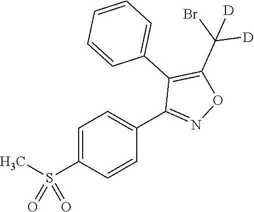 [SMILES: [2H]C([2H])(Br)c1onc(-c2ccc(S(C)(=O)=O)cc2)c1-c1ccccc1]

with a ¹⁸F source. In some embodiments, the ¹⁸F source is a ¹⁸F⁻ source.

In some embodiments, the compound of Formula I is

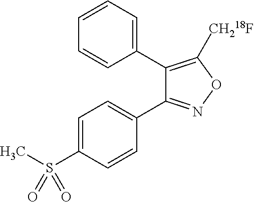

or a pharmaceutically acceptable salt thereof; and the process comprises reacting the compound

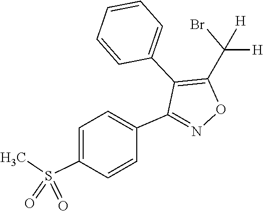 [SMILES: CS(=O)(=O)c1ccc(-c2noc(CBr)c2-c2ccccc2)cc1]

with a ¹⁸F source. In some embodiments, the ¹⁸F source is a ¹⁸F⁻ source. In some embodiments, the compound of Formula I is

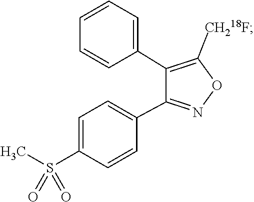

and the process comprises reacting the compound

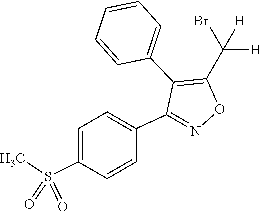 [SMILES: CS(=O)(=O)c1ccc(-c2noc(CBr)c2-c2ccccc2)cc1]

with a ¹⁸F source. In some embodiments, the ¹⁸F source is a ¹⁸F⁻ source.

In some embodiments, the compound of Formula I is

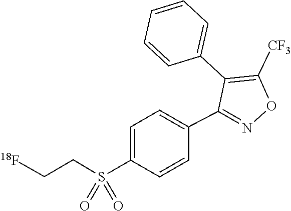 [SMILES: O=S(=O)(CC[18F])c1ccc(-c2noc(C(F)(F)F)c2-c2ccccc2)cc1]

or a pharmaceutically acceptable salt thereof; and the process comprises reacting the compound

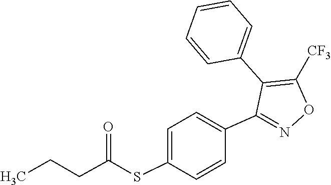 [SMILES: CCCC(=O)Sc1ccc(-c2noc(C(F)(F)F)c2-c2ccccc2)cc1]

with a ¹⁸F source. In some embodiments, the ¹⁸F source is 2-[¹⁸F]fluoroethyl tosylate. In some embodiments, the compound of Formula I is

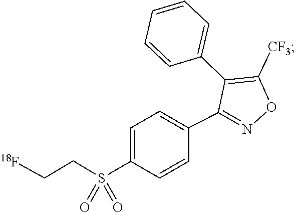 [SMILES: O=S(=O)(CC[18F])c1ccc(-c2noc(C(F)(F)F)c2-c2ccccc2)cc1]

and the process comprises reacting the compound

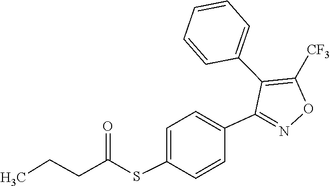 [SMILES: CCCC(=O)Sc1ccc(-c2noc(C(F)(F)F)c2-c2ccccc2)cc1]

with a ¹⁸F source. In some embodiments, the ¹⁸F source is 2-[¹⁸F]fluoroethyl tosylate.

In some embodiments, compounds of Formula I may be synthesized according to Scheme 1.

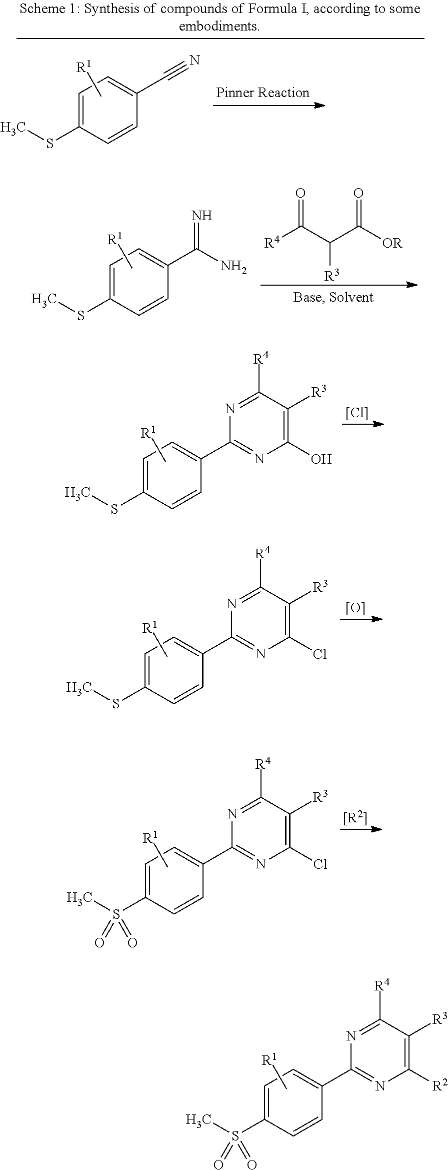

In some embodiments, the base is an alkoxide (e.g., NaOCH₃). In some embodiments, the solvent is a polar protic solvent (e.g., water, methanol, ethanol, isopropanol, and t-butanol). In some embodiments, [Cl] is a chlorinating agent (e.g., POCl₃). In some embodiments, [O] is an oxidizing agent. Non-limiting examples of oxidizing agents include Oxone®, optionally with a water/THF solvent, a peroxyacid (e.g., meto-chloroperoxybenzoic acid), potassium peroxymonosulfate, H₂O₂, NaIO₄, t-BuOCl, Ca(OCl)₂, NaClO₂, NaOCl, a dioxirane, and KMnO₄. In some embodiments, the oxidizing agent is Oxone® or potassium peroxymonosulfate. In some embodiments, the chloride is displaced with R² using, for example, a cross-coupling reaction (e.g., a palladium-mediated cross-coupling reaction) or a substitution reaction (e.g., a nucleophilic aromatic substitution reaction).

Additional processes, reagents, and conditions for synthesis of compounds of Formula I can be found in U.S. Patent Application Publication No. 2008/0138282 A1 (incorporated herein by reference in its entirety).

In some embodiments, the radiochemical yield of the reaction to install the ¹⁸F radioisotope in compounds of Formula I is about 10 to about 50%. In some embodiments, the radiochemical yield of the reaction to install the ¹⁸F radioisotope in compounds of Formula I is about 15 to about 35%. In some embodiments, the radiochemical yield of the reaction to install the ¹⁸F radioisotope in compounds of Formula I is about 20 to about 30%.

Methods of Radio Imaging

In yet another aspect, a method for imaging a sample or organism comprises:

(a) administering to the sample or organism a compound of Formula I

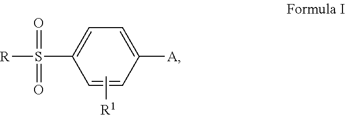

or a pharmaceutically acceptable salt thereof, in an amount effective to detect emission of deuterium, ³H, ¹¹C, ¹⁸F, ⁷⁵Br, ⁷⁶Br, ¹²⁰I, ¹²³I, ¹²⁴I, ¹²⁵I, ¹³¹I, or a combination thereof; and

(b) measuring the emission of the one or more radionuclide(s) in the compound.

In some embodiments, the amount effective to detect emission is the weight, concentration, or molar activity of the compound, or a pharmaceutically acceptable salt or dosage form thereof, that, when dosed to the sample or organism, permits detection of the radioactive emission of the radioisotope(s). In some embodiments, this is an amount that permits detection of the radioactive emission of the radioisotope(s) via PET. In some embodiments, this is an amount that permits detection of the radioactive emission of ¹⁸F. A non-limiting example of an imaging-effective dosage amount ranges from about 0.001 mCi to about 30 mCi.

In some embodiments, the emission is measured using PET.

In some embodiments, the sample is a cell culture. Non-limiting examples of cell cultures include BxPC3 (COX-2-positive) and PANC-1 (COX-2-negative).

In some embodiments, the organism is an animal. In some embodiments, the organism is a mammal. In some embodiments, the mammal is a mouse, rat, pig, dog, monkey, baboon, or human. In some embodiments, the mammal is a mouse. In some embodiments, the mammal is a rat. In some embodiments, the mammal is a pig. In some embodiments, the mammal is a dog. In some embodiments, the mammal is a monkey. In some embodiments, the monkey is a rhesus macaque. In some embodiments, the mammal is a baboon. In some embodiments, the mammal is a human.

In some embodiments, the compound can be administered to the sample or organism per se (neat) or in the form of a pharmaceutically acceptable salt or solution. For example the salt or solution may be administered by intravenous, intramuscular, or other parenteral means. The compound, salt, or solution can also be administered by intranasal application, inhalation, topically, orally, rectally, vaginally, or as implants. Suitable liquid or solid forms include, for example, aqueous or saline solutions for injection or inhalation, microencapsulated, encochleated, coated onto microscopic gold particles, contained in liposomes or other vesicles, nebulized, aerosols, pellets for implantation into the skin, or dried onto a sharp object to be scratched into the skin, granules, powders, tablets, coated tablets, (micro)capsules, suppositories, syrups, emulsions, suspensions, creams, drops, or preparations with protracted release of active compounds in whose preparation excipients and additives and/or auxiliaries include, for example, disintegrants, binders, coating agents, swelling agents, lubricants, flavorings, sweeteners or solubilizers are customarily used as described above. In some embodiments, the compound can be administered as a controlled- or sustained-release form. In some embodiments, the compound can be administered in pro-drug form. Additional administration forms and modes of administration are known in the art of pharmacy. See generally, Remington, The Science and Practice of Pharmacy, Vols. 1 and 2, Pharmaceutical Press 2013 (incorporated herein by reference in its entirety). See also U.S. Patent Application Publication No. 2008/0138282 A1 (incorporated herein by reference in its entirety).

In some embodiments, the compound interacts with a biological target and measuring the emission allows determination of the expression level or distribution of the biological target in the sample or organism. In some embodiments, the biological target (e.g., protein) is implicated in a disease (e.g., is downregulated or upregulated, is under-expressed or overexpressed, etc.) and measuring the emission allows diagnosis, progression, and treatment of the disease to be assessed. In some embodiments, the compound interacts with a protein and measuring the emission allows determination of the expression level or distribution of the protein in the sample or organism. In some embodiments, the protein is involved in inflammation and measuring the emission allows determination of the amount or distribution of inflammation, inflection, and/or injury in the sample or organism. In some embodiments, the protein is COX-2. In some embodiments, the compound interacts with (e.g., binds) and/or inhibits COX-2. In some embodiments, this binding or inhibition, in combination with the imaging method, is used to determine COX-2 expression level or activity, or diagnose or monitor a disease or treatment involving COX-2.

In some embodiments, the animal is known or suspected to have a condition or state selected from the group consisting of arthritis (e.g., rheumatoid arthritis, osteoarthritis, and spondyloarthropathies), cardiovascular disease (e.g., atherosclerosis, myocardial infarction, myocardial ischemia, periarteritis nodosa, and vascular disease), central nervous system damage (e.g., from stroke, ischemia, and trauma), central nervous system disorder (e.g., multiple sclerosis, a seizure disorder, such as epilepsy, headache, including but not limited to migraine headache, and brain injury, including but not limited to traumatic brain injury), gastrointestinal disorder (e.g., irritable bowel syndrome), hypersensitivity (e.g., autoimmune disease, including but not limited to aplastic anemia, graft rejection, lupus, scleroderma, and allergy, including but not limited to allergic rhinitis), inflammatory disease (e.g., asthma, Bechet's disease, bronchitis, bursitis, Crohn's disease, endotoxin shock syndrome, gastritis, gingivitis, inflammatory bowel disease, polymyositis, pulmonary inflammation, such as acute respiratory disease syndrome and severe acute respiratory syndrome from viral (e.g., COVID-19) and bacterial infections, Lyme disease, meningitis, and from cystic fibrosis, rheumatic fever, sarcoidosis, tendinitis, thyroiditis, and ulcerative colitis), metabolic disorder (e.g., affecting bone metabolism, type 1 diabetes, and type 2 diabetes), neoplastic disease (e.g., cancer, including but not limited to colorectal cancer, breast cancer, lung cancer, prostate cancer, bladder cancer, cervical cancer, skin cancer, and lymphoma, including but not limited to Hodgkin lymphoma and non-Hodgkin lymphoma), neurodegenerative disease (e.g., multiple sclerosis, Alzheimer's disease, amyotrophic lateral sclerosis, cortical dementias, Huntington's disease, and Parkinson's disease), neuromuscular junction disease (e.g., myasthenia gravis), ophthalmic disease (e.g., retinitis, retinopathies, uveitis, ocular photophobia, conjunctivitis, and acute injury to the eye tissue), post-operative inflammation (e.g., from ophthalmic surgery, cataract surgery, and refractive surgery), psychiatric condition (e.g., depressive disorder, schizophrenia, and an alcohol use disorder), reproductive event (e.g., ovulation, pregnancy, and child birth), respiratory disease (e.g., respiratory distress syndrome), skin disorder (e.g., psoriasis, eczema, burns, and dermatitis), tissue repair, urogenital disease (e.g., renal disease, including but not limited to nephritis and nephrotic syndrome), white matter disease, and a combination thereof.

In some embodiments, the condition is Alzheimer's disease, Parkinson's disease, rheumatoid arthritis, osteoarthritis, myocardial infarction, traumatic brain injury, inflammatory disease, cancer, renal disease, or amyotrophic lateral sclerosis.

In some embodiments, the animal is known or suspected to be experiencing pain. In some embodiments, the animal is known or suspected to be experiencing pain as a result of the conditions listed above.

In some embodiments, COX-2 is induced by inflammatory stimuli. In some embodiments, COX-2 catalyzes prostanoid formation associated with, for example, inflammation and proliferative diseases. In some embodiments, COX-2 is modestly expressed under normal physiologic conditions and plays a role in, for example, brain, cardiac, and kidney function, but is upregulated or overexpressed during inflammation. In some embodiments, excessive inflammation and associated COX-2 induction may be part of the pathogenesis of neurodegenerative diseases, such as Alzheimer's disease, Parkinson's disease, and amyotrophic lateral sclerosis, as well as in traumatic brain injury. In some embodiments, COX-2 induction is involved in pain, major depressive disorder, schizophrenia, arthritis, cancer, and acute allograft rejection. In some embodiments, inhibition of COX-2 may be a protective treatment strategy by slowing or halting the progression of disease. In some embodiments, monitoring in vivo changes in COX-2 expression can be used for quantifying disease pathogenesis, such as detecting organ rejection, detecting arthritic joints, and assessing target occupancy and biological effects of COX-2 inhibitors. In some embodiments, noninvasive radio imaging, such as PET, is useful for assessing brain diseases due to the difficulty of accessing the brain.

In some embodiments, the method can be used to measure and/or detect COX-2 protein in COX-2 associated diseases, conditions, and disorders. Non-limiting examples of COX-2-associated diseases, conditions, and disorders include a condition or state selected from the group consisting of arthritis (e.g., rheumatoid arthritis, osteoarthritis, and spondyloarthropathies), cardiovascular disease (e.g., atherosclerosis, myocardial infarction, myocardial ischemia, periarteritis nodosa, and vascular disease), central nervous system damage (e.g., from stroke, ischemia, and trauma), central nervous system disorder (e.g., multiple sclerosis, a seizure disorder, such as epilepsy, headache, including but not limited to migraine headache, and brain injury, including but not limited to traumatic brain injury), gastrointestinal disorder (e.g., irritable bowel syndrome), hypersensitivity (e.g., autoimmune disease, including but not limited to aplastic anemia, graft rejection, lupus, scleroderma, and allergy, including but not limited to allergic rhinitis), inflammatory disease (e.g., asthma, Bechet's disease, bronchitis, bursitis, Crohn's disease, endotoxin shock syndrome, gastritis, gingivitis, inflammatory bowel disease, polymyositis, pulmonary inflammation, such as acute respiratory disease syndrome and severe acute respiratory syndrome from viral (e.g., COVID-19) and bacterial infections, Lyme disease, meningitis, and from cystic fibrosis, rheumatic fever, sarcoidosis, tendinitis, thyroiditis, and ulcerative colitis), metabolic disorder (e.g., affecting bone metabolism, type 1 diabetes, and type 2 diabetes), neoplastic disease (e.g., cancer, including but not limited to colorectal cancer, breast cancer, lung cancer, prostate cancer, bladder cancer, cervical cancer, skin cancer, and lymphoma, including but not limited to Hodgkin lymphoma and non-Hodgkin lymphoma), neurodegenerative disease (e.g., multiple sclerosis, Alzheimer's disease, amyotrophic lateral sclerosis, cortical dementias, Huntington's disease, and Parkinson's disease), neuromuscular junction disease (e.g., myasthenia gravis), ophthalmic disease (e.g., retinitis, retinopathies, uveitis, ocular photophobia, conjunctivitis, and acute injury to the eye tissue), post-operative inflammation (e.g., from ophthalmic surgery, cataract surgery, and refractive surgery), psychiatric condition (e.g., depressive disorder, alcohol use disorder, and schizophrenia, reproductive event (e.g., ovulation, pregnancy, and child birth), respiratory disease (e.g., respiratory distress syndrome), skin disorder (e.g., psoriasis, eczema, burns, and dermatitis), tissue repair, urogenital disease (e.g., renal disease, including but not limited to nephritis and nephrotic syndrome), white matter disease, or a combination thereof. In some embodiments, the condition is Alzheimer's disease, Parkinson's disease, rheumatoid arthritis, osteoarthritis, myocardial infarction, traumatic brain injury, inflammatory disease, cancer, renal disease, or amyotrophic lateral sclerosis.

In some embodiments, the method can be used to detect or monitor processes, diseases, or disorders that may involve the upregulation of COX-2 protein expression including, but not limited to upregulation and/or overexpression of COX-2 is associated with inflammation, pain, fever, arthritis (e.g., rheumatoid arthritis and osteoarthritis), neurodegenerative disease (e.g., Alzheimer's disease, Parkinson's disease, amyotrophic lateral sclerosis), angiogenesis, cancer, stroke, cardiac condition (e.g., myocardial infarction and atherosclerosis), diabetes, allograft rejection, urogenital disease, renal function, tissue repair, bone metabolism, ovulation, pregnancy, and child birth.

In some embodiments, the method can be used to screen a sample or organism for diseases, disorders, states, or conditions that are related to COX-2 expression or activity. Non-limiting examples include inflammation, pain, fever, arthritis (e.g., rheumatoid arthritis and osteoarthritis), neurodegenerative disease (e.g., Alzheimer's disease, Parkinson's disease, amyotrophic lateral sclerosis), vascular condition (e.g., angiogenesis), cancer, reproduction (e.g., ovulation, pregnancy, and child birth), renal function, tissue repair, bone metabolism, stroke, cardiac condition (e.g., myocardial infarction, atherosclerosis), diabetes, allograft rejection, and urogenital disease.

In some embodiments, the method can be used to screen for organisms that are more susceptible to side effects of COX-2 inhibitors, as manifested by an increased detection of the compounds of Formula I in specified tissue compartments.

In some embodiments, the method can be used to determine the efficacy of COX-2 inhibitors administered to an organism to treat a disorder that involves the upregulation of COX-2 protein expression.

In some embodiments, the method can be used to monitor the course of inflammation in an organism. For example, whether a particular COX-2 inhibitor therapeutic regimen aimed at ameliorating the cause of the inflammatory process, or the inflammatory process itself, is effective, can be determined by measuring the decrease of COX-2 protein expression at suspected sites of inflammation.

Additional uses for the methods described herein can be found in U.S. Patent Application Publication No. 2008/0138282 A1 (incorporated herein by reference in its entirety).

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification, and guidance which can be adapted to the practice of this invention in its various embodiments and equivalents thereof.

### EXAMPLES

### Example 1: Radiosynthesis and Evaluation of [¹⁸F]FMTP and [¹⁸F]FMPP

COX, or prostaglandin endoperoxidase synthase, is an enzyme involved in the biosynthesis of prostaglandins, prostacyclins, and thromboxanes from arachidonic acid. Among the three known isoforms of COX (COX-1, COX-2, and COX-3), COX-1 has predominantly constitutive activity and is involved in many normal physiological functions. In contrast, COX-2 is inducible under normal physiologic conditions, with relatively low constitutive activity and mostly found in kidney, brain, and heart. The third isoform, COX-3, may be responsible for febrile response and mediates the antipyretic effects of aspirin and acetaminophen. COX-2 inhibition mediates the analgesic activities of NSAIDs. COX-2, induced by inflammatory stimuli, catalyzes prostanoid formation associated with inflammation and proliferative diseases, including those in the CNS. Neuroinflammation and COX-2 induction are implicated in the pathogenesis of neurodegenerative diseases such as Alzheimer's disease, Parkinson's disease, and ALS, as well as psychiatric disorders, smoking, seizure disorders, and brain injuries (e.g., TBI). COX-2 induction is also involved in for example, pain, arthritis, cancers, myocardial infarction, and acute allograft rejection. COXIBs can have anti-inflammatory effects.

Upregulation/overexpression of COX-2 is involved in neuroinflammation associated with many neurological diseases and malignancies of the brain. Outside the brain, inflammation and COX-2 induction contribute to the pathogenesis of, for example, pain, arthritis, and acute allograft rejection, and to responses to, for example, infections, tumors, autoimmune disorders, and injuries. Therefore, targeting COX-2 may be a potential neuroprotective treatment strategy aiming to reduce the progression of neurodegenerative and other diseases.

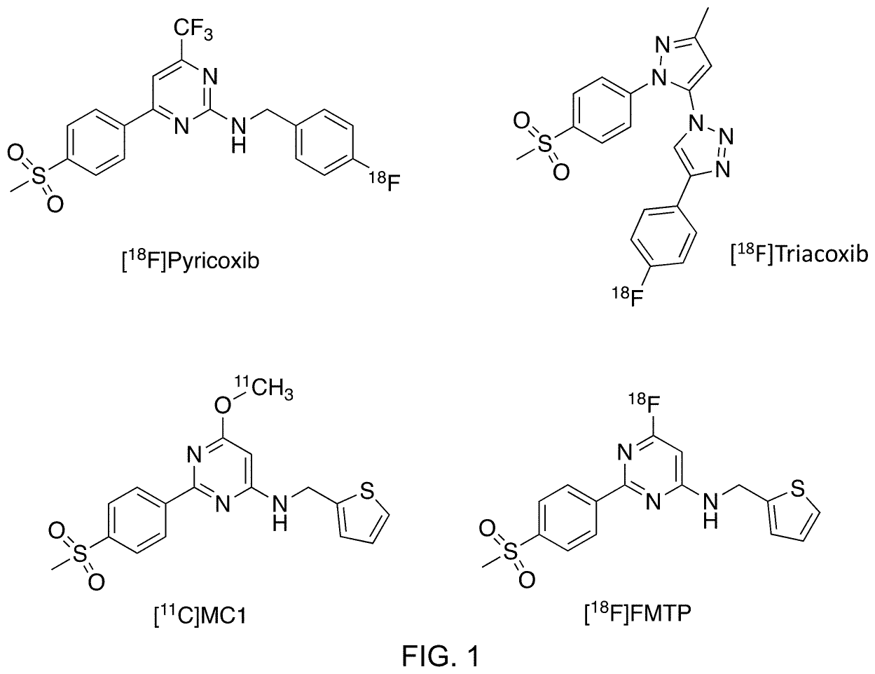

Monitoring in vivo changes in COX-2 expression allows, for example, quantification of inflammation, tracking of disease course, assessing target occupancy of NS AIDs, and or monitoring clinical use of FDA-approved COXIB medications. Prior methods of COX-2 quantification include ex vivo assays of tissue samples, invasively obtained from biopsies. PET imaging would allow noninvasive and in vivo visualization of COX-2 throughout the body. Prior COX-2 PET ligands were not successful for in vivo quantification of COX-2 due to limitations, such as suboptimal COX-2 affinity, high nonspecific binding, de[¹⁸F]fluorination, skeletal uptake, poor brain or organ uptake, inability to detect basal or low level of COX-2, and poor signal-to-noise ratio due to lipophilicity. [¹¹C]TMI, a potent COX-2 inhibitor, was the only prior radiotracer exhibiting partial blocking of constitutive COX-2 level in baboon brain, however, its ability to image inflammation in animal disease models was yet to be proven. [¹⁸F]Pyricoxib, another prior COX-2 inhibitor belonging to the class of 2-(4-methylsulfonylphenyl)pyrimidines, and the prior triazole analogue [¹⁸F]triacoxib, showed a higher binding in COX-2 positive cells (HCA-7) compared with COX-2 negative cells (HCT-116). However, both tracers demonstrated only modest binding in xenografts in vivo and did not show significant uptake in brain. See FIG. 1. Similarly, [¹¹C]MC1, another member of the class of arylpyrimidines, showed increased binding to COX-2 in animal models of neuroinflammation. See FIG. 1. Despite promising uptake in neuroinflammation, [¹¹C]MC1 failed to detect constitutive or low level of COX-2 in brain under conditions without inflammation, and cold (i.e., non-radiolabeled) MC1 showed no specific binding in CNS and periphery organs. COXIBs with sub-nanomolar affinity and radiolabeled with long-lived isotopes, such as ¹⁸F (decay half-life 110 min), may overcome the limitations of these prior tracers. In Example 1, the radiosynthesis and evaluation of [¹⁸F]FMTP; COX-2 IC₅₀=2.5 nM) as potential PET ligand for COX-2 imaging in brain and periphery (see FIG. 1) was conducted. [¹⁸F]FMTP, belonging to the pyrimidine class of COXIBs, was evaluated as a PET tracer for COX-2 imaging owing to the presence of a chemically and metabolically stable radiolabeling position on the aromatic ring, level of lipophilicity for passive brain entry, and high COX-2 affinity.

Materials and Methods

The radiochemical synthesis of [¹⁸F]FMTP and [¹⁸F]FMPP were optimized using a chlorine-to-fluorine displacement method, by reacting ¹⁸F-fluoride/K222/K₂CO₃ with the corresponding chlorine precursor molecules. Cellular uptake studies of [¹⁸F]FMTP were performed in COX-2-positive BxPC3 and COX-2-negative PANC-1 cell lines with unlabeled FMTP as well as celecoxib (5 μM) to define specific binding agents. Dynamic microPET image acquisition was performed in anesthetized nude mice, LPS-induced neuroinflammation mice, and PBS-administered control mice using a Trifoil mPET/CT for a scan period of 50 or 60 minutes.

Materials

¹⁸F was produced using Eclipse cyclotron (Siemens, Knoxville, Tenn.). Gamma-ray detector (Bioscan Flow-Count fitted with NaI detector) coupled in series with a UV detector (Waters Model 996 set at 254 nm) were used for detection of radiolabeled products. Data acquisition for both the analytical and preparative systems was accomplished using a Waters Empower Chromatography System. Dynamic microPET image acquisitions were performed with Trifoil mPET/CT scanner.

Synthesis of [¹⁸F]FMTP and [¹⁸F]FMPP

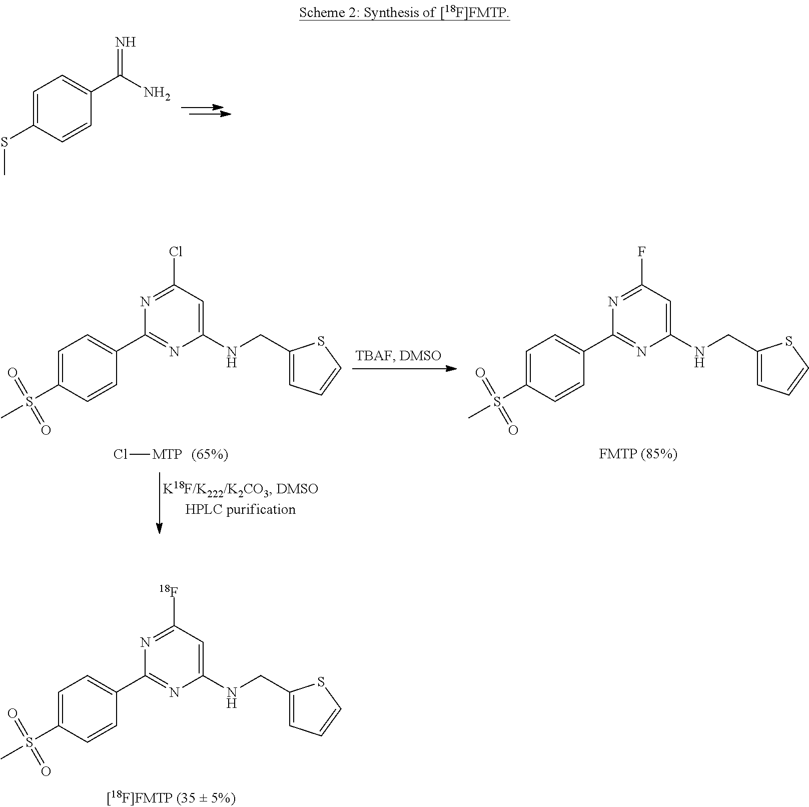

Synthesis of the starting 4-(methylthio)benzimidamide was conducted as reported in Orjales et al. “Novel 2-(4-Methylsulfonylphenyl)pyrimidine Derivatives as Highly Potent and Specific COX-2 Inhibitors. Bioorg. Med. Chem. 2008, 16:2183-99 (incorporated herein by reference in its entirety. See also Scheme 1.

Thiophene-2-ylmethanamine (68 mg, 0.6 mmol) was added to a solution of 4,6-dichloro-2-(4-(methylsulfonyl)phenyl)pyrimidine (0.3 mmol, 90 mg) in 4 mL dry dichloromethane and 0.1 mL triethylamine. The reaction mixture was stirred at room temperature for 1 hour and, at this time, HPLC analyses showed >95% consumption of the chloro-substrate. The reaction mixture was evaporated and chromatographed over silica gel using 40:60 ethyl acetate-hexane to afford 75 mg (65%) of Cl-MTP as pale-yellow solid. Cl-MTP: melting point: 169-171° C.; ¹H NMR (400 MHz, CDCl₃): δ 3.0 (3H, s, CH₃), 4.85 (2H, bs, CH₂), 5.3 (1H, bs, NH), 6.3 (1H, s), 6.9 (1H, m), 7.0 (1H, d, J=2.81 Hz), 7.2 (2H, d, overlapped with CDCl₃), 7.9 (2H, d, J=8.53 Hz), and 8.5 (2H, d, J=8.56 Hz).

Tetra-n-butylammonium fluoride (“TBAF”) (0.1 mL, 1 M solution in THF) was added to a solution of Cl-MTP (20 mg, 0.05 mmol) in DMF (2 mL). The resulting solution was heated at 140° C. for 1 hour, at which time point, HPLC analyses showed >95% consumption of Cl-MTP. The reaction mixture was then allowed to cool, diluted with water (20 mL), and extracted with ethyl acetate (3×10 mL). The combined ethyl acetate fractions were further washed with saturated brine, dried over anhydrous MgSO₄, and chromatographed over silica gel (20% ethyl acetate-hexane) to afford 15 mg (85%) of FMTP as pale-yellow solid. FMTP: melting point: 131.5° C.; ¹H NMR (400 MHz, CDCl₃): δ 3.0 (3H, s, CH₃), 4.8 (2H, bs, CH₂), 5.9 (1H, bs, NH), 6.4 (1H, s), 6.9-7 (1H, m), 7.05 (1H, d, J=2.82 Hz), 7.2 (2H, d, overlapped with CDCl₃), 8.0 (2H, d, J=8.51), 8.6 (2H, d, J=8.54); HRMS (MH+) calculated for: C₁₆H₁₅FN₃O₂S₂: 364.0512; Found: 364.0533.

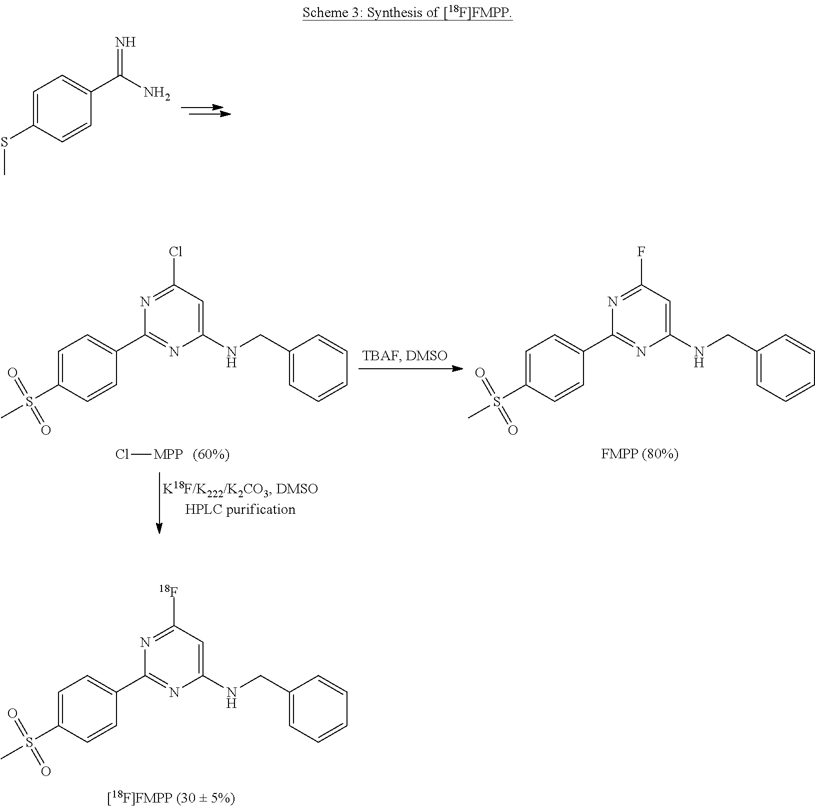

Benzylamine (64 mg, 0.6 mmol) was added to a solution of 4,6-dichloro-2-(4-(methylsulfonyl)phenyl)pyrimidine (0.3 mmol, 90 mg) in 4 mL dry di chloromethane and 0.1 mL triethylamine. The reaction mixture was stirred at room temperature for 1 hour and, at this time, HPLC analyses showed >95% consumption of the chloro-substrate. The reaction mixture was evaporated and chromatographed over silica gel using 40:60 ethyl acetate-hexane to afford 65 mg (60%) of Cl-MPP as pale-yellow solid.

TBAF (0.1 mL, 1 M solution in THF) was added to a solution of Cl-MPP (20 mg, 0.05 mmol) in DMF (2 mL). The resulting solution was heated at 140° C. for 1 hour, at which time point, HPLC analyses showed >95% consumption of Cl-MTP. The reaction mixture was then allowed to cool, diluted with water (20 mL), and extracted with ethyl acetate (3×10 mL). The combined ethyl acetate fractions were further washed with saturated brine, dried over anhydrous MgSO₄, and chromatographed over silica gel (20% ethyl acetate-hexane) to afford 13 mg (80%) of FMPP as pale-yellow solid. FMPP: melting point: 125.5° C.; ¹H NMR (400 MHz, CDCl₃): δ 3.0 (3H, s, CH₃), 4.9 (2H, bs, CH₂), 6.1 (1H, bs, NH), 6.5 (1H, s), 7.3 (5H, m), 8.0 (2H, d, J=8.51), 8.6 (2H, d, J=8.54); HRMS (MH+) calculated for: C₁₈H₁₇FN₃O₂S: 357.0942; Found: 357.0926.

¹⁸F (Eclipse cyclotron, Siemens) trapped from QMA was eluted with 1 mL of 10:1 acetonitrile:water, containing K₂₂₂ (36 mg) and potassium carbonate (2 mg). The reaction mixture was azeotropically heated and dried at 98° C. under a stream of argon by the repeated addition of acetonitrile (4×0.5 mL). A solution of approximately 2 mg of Cl-MTP or Cl-MPP in 500 μL of DMSO was then added to the reaction vial, sealed, and heated for 20 minutes at 140° C. The reaction mixture was allowed to cool to room temperature, diluted with 20 mL water, and passed through a classic C-18 Sep-Pak cartridge and eluted with 1 mL acetonitrile. The crude product in acetonitrile was injected onto a semipreparative HPLC column (Phenomenex, Prodigy ODS-Prep 10×250 mm, 10 μm; and eluted with 50:50 acetonitrile:0.1 M ammonium formate with a flow rate of 8 mL/min). [¹⁸F]FMTP eluted at 9-10 minutes and [¹⁸F]FMPP eluted at 12 minutes, and were collected based on the γ-detector reading, diluted with 50 mL of deionized water and passed through a classic C-18 Sep-Pak cartridge, washed with 10 mL of deionized water, and eluted with 1 mL of ethanol. Reconstruction of the product in 1 mL of absolute ethanol afforded [¹⁸F]FMTP in 35±5% yield and [¹⁸F]FMPP at 30±5% at the end of synthesis (“EOS”). A portion of the ethanol solution was analyzed by analytical HPLC (Phenomenex, Prodigy ODS(3) 4.6×250 mm, 5 μm; mobile phase; 60:40 acetonitrile:0.1 M AMF, flow rate 2 mL/min, tR=˜5-6 minutes) to determine the molar activity, chemical, and radiochemical purities. The ethanol solution was then diluted to a volume of 10 mL with saline and filtered through a sterile environment, and a portion of this solution was formulated for injection.

In Vitro and In Vivo Evaluation of [¹⁸F]FMTP

Cell Update of [¹⁸F]FMTP

BxPC3 and PANC-1 human pancreas carcinoma cell lines were plated on a 24-well plate at 2×10⁵ cells/well. After 48 hours, [¹⁸F]FMTP was added (2.0 μCi/mL) to the cell medium for 30 minutes. For blocking experiments, non-labelled FMTP or celecoxib (5 μM) was added to cells 30 minutes before [¹⁸F]FMTP. After incubation with [¹⁸F]FMTP, cells were washed 4 times with cold PBS, lysed with 0.1 N NaOH, and counted in a gamma counter (Hidex AMG, LabLogic, Tampa, Fla.).

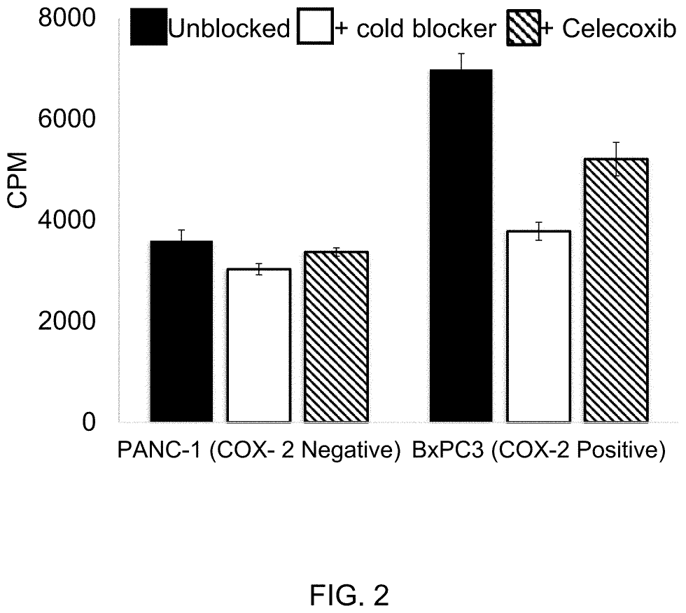

Proof-of-concept and selectivity of [¹⁸F]FMTP binding to COX-2 was examined by evaluating the tracer uptake in COX-2-positive BxPC3 and a control COX-2-negative PANC1 human pancreatic carcinoma cell lines. FIG. 2 illustrates a >2-fold uptake of [¹⁸F]FMTP in BxPC3 cells compared with PANC1 cells. Values shown in FIG. 2 are the mean±standard deviation (“SD”) from four independent experiments. This binding was substantially blocked with unlabeled FMTP. Blocking with celecoxib, an FDA-approved COXIB, produced less proportional blocking compared with cold FMTP. [¹⁸F]FMTP showed negligible binding to PANC1 cells and no specific binding with FMTP or celecoxib block.

MicroPET Imaging of [¹⁸F]FMTP

Animals (male white mice) were anesthetized with isoflurane (l %-2% isoflurane in 100% oxygen) using a nose cone, and a 29-gauge needle connected to a catheter was placed into the lateral tail vein. For neuroinflammation, mice were stereotactically injected with 5 μg of LPS or PBS (controls) in the brain and imaging experiments conducted 24 hours later. The animal was placed in prone position on the platform of the scanner and moved into the center of the field of view guided by laser beam calibration. Immediately after scan start, [¹⁸F]FMTP (˜2.5 MBq 25 mL in 20% ethanol-saline solution) was injected through the tail-vein catheter manually. The error caused by the injector and catheter was corrected by subtracting the remaining dose. 40-minute dynamic imaging was acquired on a microPET scanner (Siemens Inveon). The acquired list-mode data were reconstructed with 3D ordered subset expectation maximization (OSEM) algorithm using the software of Siemens Inveon Acquisition Workplace with a framing protocol of 2×30 seconds, 4×60 seconds, 3×120 seconds, 3×180 seconds, and 4×300 seconds. Using PMOD software (version 4.0, Switzerland), three-dimensional ellipsoid volume of interests (“VOIs”) ranging from 2-to-6 mm were placed manually at the center of the brain, heart (blood-pool), liver, proximal humerus (bone), and posterior cervical muscle. The standardized uptake values (“SUVs”) were estimated using a calibration factor calculated from the phantom study and time activity curves (“TACs”) were derived from VOIs in the series of reconstructed images.

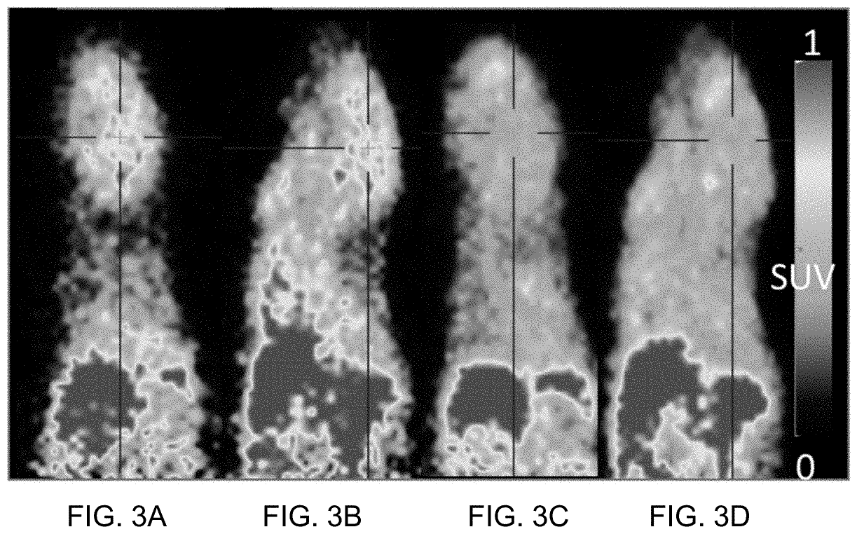

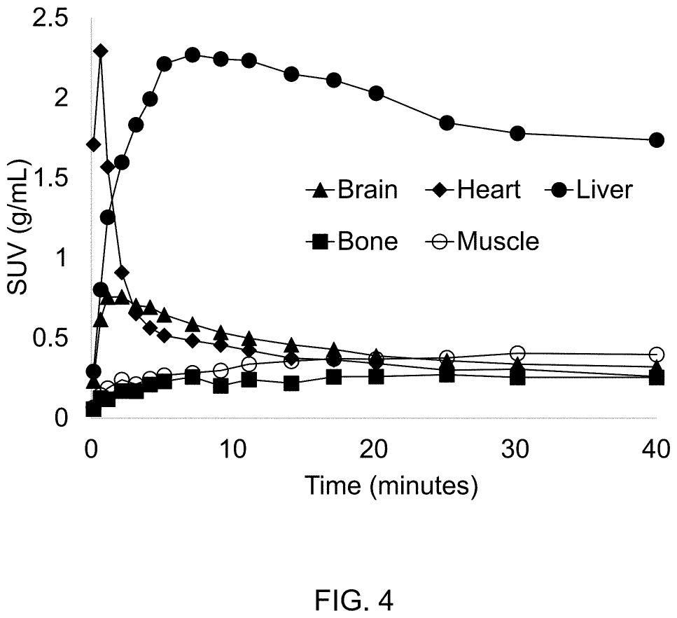

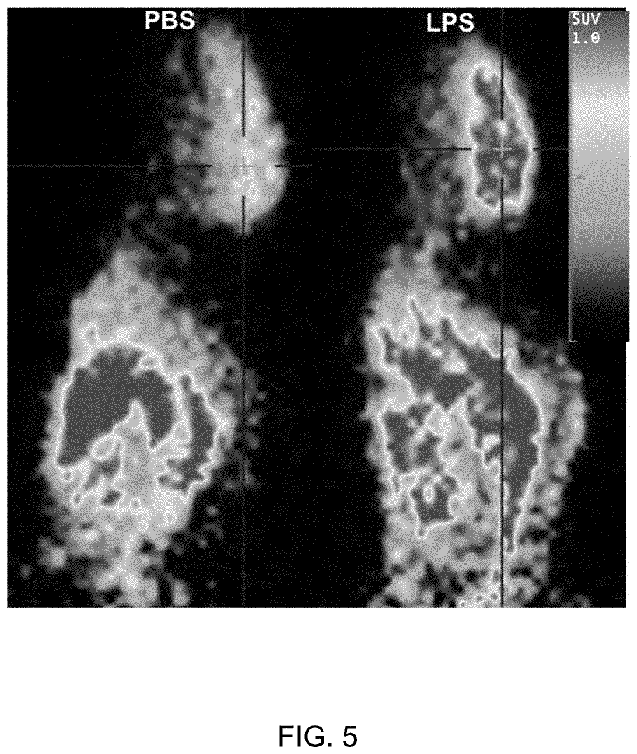

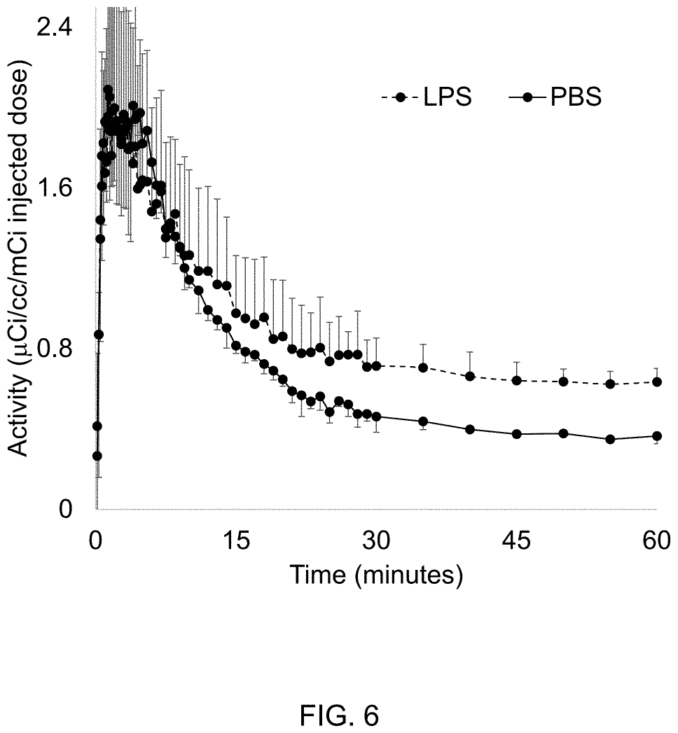

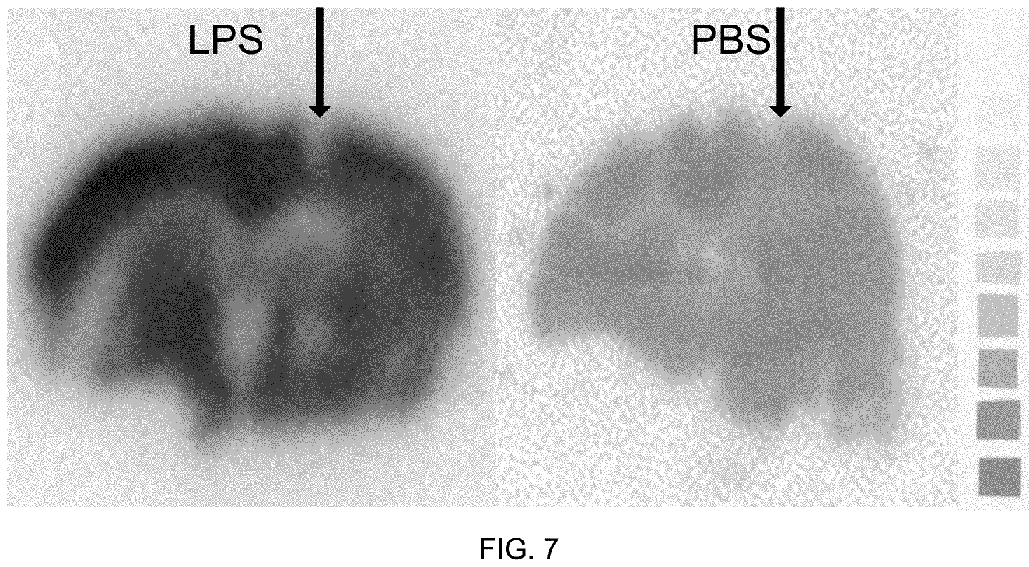

FIGS. 3A-3D show the summed early frames (0-10 minute) and total frames (0-40 minute) of microPET images. Crosses in FIGS. 3A-3D represent the region with brain. As evident from the images, the tracer penetrated the BBB and subsequently showed a fast washout of activity from brain. TACs also indicated an initial rapid influx of radioactivity followed by rapid washout (FIG. 4). The uptake of [¹⁸F]FMTP peaked around 1 minute in heart and brain, and then decreased rapidly. The absence of retention of [¹⁸F]FMTP activity in these organs was predicted due to low COX-2 expression in normal mouse brain. Highest radioactivity outside the heart was found in liver, and the delay to peak at 5-10 minutes was probably due to the accumulation of radioactive metabolite(s). The tracer did not show uptake in spine and skeleton, which indicated lack of de[¹⁸F]fluorination, an advantage of [¹⁸F]FMTP for use in in vivo PET imaging. The binding of [¹⁸F]FMTP in LPS-induced neuroinflammation in mice was studied in comparison to vehicle (PBS)-treated-mice. Intracranial injection of LPS in mice is known to generate COX-2 induction and neuroinflammation after around 24 hours. Therefore, PET imaging of LPS treated mice was performed after 24 hours with [¹⁸F]FMTP, and an approximately 2-fold increase of tracer binding was found in brain compared with PBS-treated mice (FIG. 5). TACs in whole brain showed higher binding of [¹⁸F]FMTP in LPS-treated mice compared with PBS-treated controls (FIG. 6). MicroPET data correlated with autoradiography performed in brain sections of microPET imaged animals using [¹⁸F]FMTP. See FIG. 7.

A new method for radiosynthesis of [¹⁸F]FMTP and [¹⁸F]FMPP included a chlorine-to-¹⁸F displacement reaction of aryl[1,3]pyrimidine core molecule. Advantages of the radiosynthesis described in one or more embodiments herein include, but are not limited to, accessibility of precursor, one step, no prosthetic group required, facile separation and purification, and no de[¹⁸F]fluorination. Proof-of-concept of the use of [¹⁸F]FMTP for quantifying COX-2 was demonstrated first, in vitro, in COX-2-positive BxPC3 cells. MicroPET imaging of normal mice demonstrated BBB penetration and a fast washout of radioactivity from brain, possibly due to low concentration of COX-2 in normal brain. [¹⁸F]FMTP showed a higher binding in LPS-induced neuroinflammation compared to binding in the brain of control mice. Specific binding to COX-2 in cell lines, lack of in vivo de[¹⁸F]fluorination and skeletal uptake, BBB permeability, and higher brain binding in neuroinflammation demonstrated the potential of [¹⁸F]FMTP as a PET tracer for imaging inflammation where COX-2 overexpression is reported. [¹⁸F]FMTP may be useful for rapid determination of the lowest effective dose of, e.g., a COXIB.

### REFERENCES

Fitzpatrick F. “Cyclooxygenase enzymes: regulation and function. Curr. Pharm. Des. 2004, 10(6):577-88 (incorporated herein by reference in its entirety).
DeWitt D L. “Prostaglandin endoperoxide synthase: regulation of enzyme expression.” Biochim. Biophys. Acta. 1991, 1083(2): 121-34 (incorporated herein by reference in its entirety).
Smith W L et al. “Prostaglandin endoperoxide synthase: structure and catalysis.” Biochim. Biophys. Acta. 1991, 1083(1): 1-17 (incorporated herein by reference in its entirety).
Kumata K et al. “Assessment of radioligands for PET imaging of cyclooxygenase-2 in an ischemic neuronal Injury model.” Brain Res. 2013, 1533:152-62 (incorporated herein by reference in its entirety).
Yasojima K et al. “Distribution of cyclooxygenase-1 and cyclooxygenase-2 mRNAs and proteins in human brain and peripheral organs.” Brain Res. 1999, 830(2):226-36 (incorporated herein by reference in its entirety).
Ho L et al. “Regional distribution of cyclooxygenase-2 in the hippocampal formation in Alzheimer's disease.” J Neurosci. Res. 1999, 57(3):295-303 (incorporated herein by reference in its entirety).
Dotting R et al. “COX-3 and the mechanism of action of paracetamol/acetaminophen.” Prostaglandins Leukot Essent Fatty Acids 2005, 72(2): 85-7 (incorporated herein by reference in its entirety).
Schwab J M et al. “COX-3 the enzyme and the concept: steps towards highly specialized pathways and precision therapeutics?” Prostaglandins Leukot Essent Fatty Acids 2003, 69(5):339-43 (incorporated herein by reference in its entirety).
Warner T D et al. “Cyclooxygenase-3 (COX-3): filling in the gaps toward a COX continuum?” Proc. Nad. Acad. Sci. USA 2002, 99(21): 13371-3 (incorporated herein by reference in its entirety).
Choi S H et al. “The distinct roles of cyclooxygenase-1 and -2 in neuroinflammation: implications for translational research.” Trends Pharmacol Sci. 2009, 30(4): 174-81 (incorporated herein by reference in its entirety).
Minghetti L. “Cyclooxygenase-2 (COX-2) in inflammatory and degenerative brain diseases.” J. Neuropathol. Exp. Neurol. 2004, 63(9):901-10 (incorporated herein by reference in its entirety).
Guan P P et al. “Integrated communications between cyclooxygenase-2 and Alzheimer's disease.” FASEB J. 2019, 33(1): 13-33 (incorporated herein by reference in its entirety).
Dhir A. “An update of cyclooxygenase (COX)-inhibitors in epilepsy disorders.” Expert Opin. Investig. Drugs. 2019, 28(2): 191-205 (incorporated herein by reference in its entirety).
Rojas A et al. “The COX-2/prostanoid signaling cascades in seizure disorders.” Expert Opin. Ther. Targets. 2019, 23(1): 1-13 (incorporated herein by reference in its entirety).
Strauss K I. “COX2 inhibitors for acquired brain injuries: Is the time ripe?” Crit. Care. Med. 2010, 38(2):723-724 (incorporated herein by reference in its entirety).
de Vries E F et al. “Nuclear imaging of inflammation in neurologic and psychiatric disorders.” Curr. Clin. Pharmacol. 2006, 1(3):229-42 (incorporated herein by reference in its entirety).
Fond G et al. “Effectiveness and tolerance of anti-inflammatory drugs' add-on therapy in major mental disorders: a systematic qualitative review.” Acta. Psychiatr. Scand. 2014, 129(3): 163-79 (incorporated herein by reference in its entirety).
Na K S et al. “The role of pro-inflammatory cytokines in the neuroinflammation and neurogenesis of schizophrenia.” Prog. Neuropsychopharmacol. Biol. Psychiatry. 2014, 48:277-86 (incorporated herein by reference in its entirety).
Mathew S T et al. “Efficacy and safety of COX-2 inhibitors in the clinical management of arthritis: mini review” ISRN Pharmacol. 2011, 2011:480291 (incorporated herein by reference in its entirety).
Yang X et al. “Upregulation of COX-2 during cardiac allograft rejection.” Circulation 2000, 101(4):430-8 (incorporated herein by reference in its entirety).
Patti R et al. “Overexpression of cyclooxygenase-2 (COX-2) in human primitive neuroectodermal tumors: effect of celecoxib and rofecoxib.” Cancer Lett. 2002, 180(1): 13-21 (incorporated herein by reference in its entirety).
Puljak L et al. “Celecoxib for osteoarthritis.” Cochrane Database Syst. Rev. 2017, 5:CD009865 (incorporated herein by reference in its entirety).
Rayar A M et al. “Update on COX-2 selective inhibitors: chemical classification, side effects and their use in cancers and neuronal diseases.” Curr. Top. Med. Chem. 2017, 17(26):2935-56 (incorporated herein by reference in its entirety).
Miao X P et al. “Role of selective cyclooxygenase-2 inhibitors in exacerbation of inflammatory bowel disease: a systematic review and meta-analysis.” Curr. Ther. Res. 2008, 69(3): 181-91 (incorporated herein by reference in its entirety).
Prabhakaran J et al. “Synthesis of [11C]celecoxib: A potential PET probe for imaging COX-2 expression.” J. Label. Comp. Radiopharm. 2005, 48:887-95 (incorporated herein by reference in its entirety).
Prabhakaran J et al. “Synthesis and in vivo evaluation of [18F]-4-[5-(4-methylphenyl)-3(trifluoromethyl)-1H-pyrazol-1-yl]benzenesulfonamide as a PET imaging probe for COX-2 expression.” Bioorg. Med. Chem. Lett. 2007, 15:1802-7 (incorporated herein by reference in its entirety).
Toyokuni T et al. “Synthesis of 4-(5-[18F]fluoromethyl-3phenylisoxazol-4-yl)benzenesulfonamide, a new [18F]fluorinated analogue of valdecoxib, as a potential radiotracer for imaging cyclooxygenase-2 with positron emission tomography.” Bioorg. Med. Chem. Lett. 2005, 15:4699-702 (incorporated herein by reference in its entirety).
Prabhakaran J et al. “Radiosynthesis and in vivo evaluation of [11C]MOV as a PET imaging agent for COX-2.” Bioorg. Med. Chem. Lett. 2018, 28(14):2432-5 (incorporated herein by reference in its entirety).
Kaur J et al. “Design, synthesis, and evaluation of an 18F-labeled radiotracer based on celecoxib-NBD for positron emission tomography (PET) imaging of cyclooxygenase-2 (COX-2).” Chem. Med. Chem. 2015, 10:1635-40 (incorporated herein by reference in its entirety).
Wuest F et al. “Synthesis and evaluation in vitro and in vivo of a 11C-labeled cyclooxygenase-2 (COX-2) inhibitor.” Bioorg. Med. Chem. 2008, 16:7662-70 (incorporated herein by reference in its entirety).
De Vries E F J et al. “Synthesis and in vivo evaluation of 18F-desbromo-DuP-697 as a PET tracer for cyclooxygenase-2 expression.” J. Nucl. Med. 2003, 44:1700-6 (incorporated herein by reference in its entirety).
De Vries E F J et al. “Evaluation of [11C]rofecoxib as PET tracer for cyclooxygenase 2 overexpression in rat models of inflammation.” Nucl. Med. Biol. 2008, 35:35-42 (incorporated herein by reference in its entirety).
Tanaka M et al. “Radiosynthesis and evaluation of 11C-labeled diaryl-substituted imidazole and indole derivatives for mapping cyclooxygenase-2.” Biol. Pharm. Bull. 2006, 29:2087-209 (incorporated herein by reference in its entirety).
Pacelli A et al. “Imaging COX-2 expression in cancer using PET/SPECT radioligands: current status and future directions.” J Labelled. Comp. Radiopharm. 2014, 57(4): 317-22 (incorporated herein by reference in its entirety).
Tietz O et al. “Radiotracers for molecular imaging of cyclooxygenase-2 (COX-2) enzyme.” Curr. Med. Chem. 2013, 20(35):4350-69 (incorporated herein by reference in its entirety).
Kumar J S D et al. “In vivo evaluation of [11C]TMI, a COX-2 selective PET tracer, in baboons.” Bioorg. Med. Chem. Lett. 2018, 28(23-24):3592-5 (incorporated herein by reference in its entirety).
Tietz O et al. “PET imaging of cyclooxygenase-2 (COX-2) in a pre-clinical colorectal cancer model.” Eur. J. Nuc. Med. Mol. Imag. Res. 2016, 6(1):37 (incorporated herein by reference in its entirety).
Tietz O et al. “Impact of structural alterations on the radiopharmacological profile of 18F-labeled pyrimidines as cyclooxygenase-2 (COX-2) imaging agents.” Nucl. Med. Biol. 2018, 62-63:9-17 (incorporated herein by reference in its entirety).
Litchfield M et al. “Radiosynthesis and biological evaluation of [18F]triacoxib: a new radiotracer for PET imaging of COX-2.” Mol. Pharm. 2020, 17(1):251-61 (incorporated herein by reference in its entirety).
Kim M-J et al. “Evaluation of two potent and selective PET radioligands to image COX-1 and COX-2 in rhesus monkeys.” J. Nucl. Med. 2018, 59(12): 1907-12 (incorporated herein by reference in its entirety).
Gallagher E et al. “Novel PET radioligands show That COX-2, but not COX-1, is induced by neuroinflammation in rhesus macaque.” Biological Psychiatry Annual Meeting, 2018, 83(9):S160 (incorporated herein by reference in its entirety).
Prabhakaran J et al. “Radiosynthesis and evaluation of [18F]FMTP: a potential COX-2 PET ligand.” J. Nucl. Med. 2019, 60:1106 (incorporated herein by reference in its entirety).
Cortes-Salva M Y et al. “2-(4-Methylsulfonylphenyl)pyrimidines as prospective radioligands for imaging cyclooxygenase-2 with PET-synthesis, triage, and radiolabeling.” Molecules 2018, 23(11):2850 (incorporated herein by reference in its entirety).
Orjales A et al. “Novel 2-(4-methylsulfonylphenyl)pyrimidine derivatives as highly potent and specific COX-2 inhibitors.” Bioorg. Med. Chem. 2008, 16(5):2183-99 (incorporated herein by reference in its entirety).
Catorce M N et al. “LPS-induced murine neuroinflammation model: main features and suitability for pre-clinical assessment of nutraceuticals.” Curr. Neuropharmacol. 2016, 14(2): 155-64 (incorporated herein by reference in its entirety).
Font-Nieves M et al. “Induction of COX-2 enzyme and down-regulation of COX-1 expression by lipopolysaccharide (LPS) control prostaglandin E2 production in astrocytes.” J. Biol. Chem. 2012, 287(9):6454-68 (incorporated herein by reference in its entirety).
Yao R et al. “Performance characteristics of the 3-D OSEM algorithm in the reconstruction of small animal PET images. Ordered-subsets expectation-maximization.” IEEE transactions on medical imaging. 2000, 19, 798-804 (incorporated herein by reference in its entirety).
Yang Z et al. “Dynamic FDG-PET imaging to differentiate malignancies from inflammation in subcutaneous and in situ mouse model for non-small cell lung carcinoma (NSCLC).” PLoS One 2015, 10(9):e0139089 (incorporated herein by reference in its entirety).

1. A compound of Formula I: 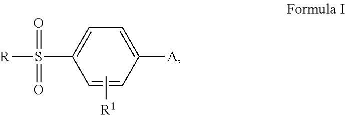
or a pharmaceutically acceptable salt thereof,
wherein:
A is 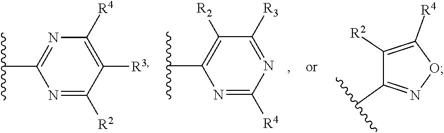
R is selected from the group consisting of (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, (C₁-C₆)alkyl-heteroaryl, and N(R⁵)₂;
wherein each (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, or (C₁-C₆)alkyl-heteroaryl is optionally substituted with one or more deuterium, ³H, ¹¹C, halogen, ¹⁸F, ⁷⁵Br, ⁷⁶Br, ¹²⁰I, ¹²³I, ¹²⁴I, ¹²⁵I, ¹³¹I, N(R⁵)₂, CN, OR⁵, SR⁵, SOR⁵, SO₂R⁵, SO₂NHR⁵, SO₃R⁵, NHSO₂R⁵, COR⁵, or NHCOR⁵;
R¹, R², R³, and R⁴ are each independently selected from the group consisting of hydrogen, deuterium, ³H, ¹¹C, halogen, ¹⁸F, ⁷⁵Br, ⁷⁶Br, ¹²⁰I, ¹²³I, ¹²⁴I, ¹²⁵I, ¹³¹I, (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, (C₁-C₆)alkyl-heteroaryl, N(R⁵)₂, CN, OR⁵, SR⁵, SOR⁵, SO₂R⁵, SO₂NHR⁵, SO₃R⁵, NHSO₂R⁵, COR⁵, and NHCOR⁵;
wherein each (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, or (C₁-C₆)alkyl-heteroaryl is optionally substituted with one or more deuterium, ³H, ¹¹C, halogen, ¹⁸F, ⁷⁵Br, ⁷⁶Br, ¹²⁰I, ¹²³I, ¹²⁴I, ¹²⁵I, ¹³¹I, N(R⁵)₂, CN, OR⁵, SR⁵, SOR⁵, SO₂R⁵, SO₂NHR⁵, SO₃R⁵, NHSO₂R⁵, COR⁵, or NHCOR⁵; and
each occurrence of R⁵ is independently selected from the group consisting of hydrogen, deuterium, ³H, (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, and (C₁-C₆)alkyl-heteroaryl;
wherein each (C₁-C₆)alkyl, (C₁-C₆)alkenyl, (C₁-C₆)alkynyl, (C₁-C₆)heteroalkyl, (C₁-C₆)heteroalkenyl, (C₁-C₆)heteroalkynyl, (C₃-C₇)cycloalkyl, (C₃-C₇)heterocycloalkyl, (C₃-C₇)cycloalkenyl, (C₃-C₇)heterocycloalkenyl, (C₁-C₆)alkyl-(C₃-C₇)cycloalkyl, (C₁-C₆)alkyl-(C₃-C₇)heterocycloalkyl, (C₁-C₆)alkyl-(C₃-C₇)cycloalkenyl, (C₁-C₆)alkyl-(C₃-C₇)heterocycloalkenyl, aryl, (C₁-C₆)alkyl-aryl, heteroaryl, and (C₁-C₆)alkyl-heteroaryl is optionally substituted with one or more deuterium, ³H, ¹¹C, ¹⁸F, ⁷⁵Br, ⁷⁶Br, ¹²⁰I, ¹²³I, ¹²⁴I, ¹²⁵I, or ¹³¹I;
wherein at least one of R, R¹, R², R³, R⁴, or R⁵ comprises at least one of ³H, ¹¹C, ¹⁸F, ⁷⁵Br, ⁷⁶Br, ¹²⁰I, ¹²³I, ¹²⁴I, ¹²⁵I, or ¹³¹I;
wherein when A is 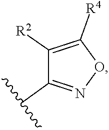
R is ¹¹CH₃, R¹ is hydrogen, and R² is phenyl, R⁴ is not CF₃;
wherein when A is 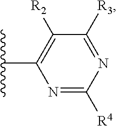
R is CH₃, R¹ and R² are hydrogen, and R³ is CF₃, R⁴ is not 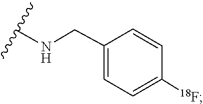
and
wherein when A is 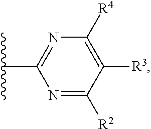
R is CH₃, R¹ and R³ are hydrogen, and R⁴ is O¹¹CH₃, R² is not

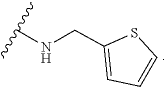 [SMILES: *NCc1cccs1]

2. The compound of claim 1, wherein A is

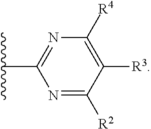

3. The compound of claim 1, wherein A is

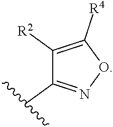

4. The compound of claim 1, wherein A is

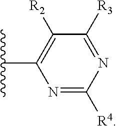

5. The compound of claim 1, wherein R is selected from the group consisting of (C₁-C₆)alkyl, (C₁-C₆)alkynyl, (C₁-C₆)heteroalkyl, (C₁-C₆)heteroalkynyl, (C₃-C₇)cycloalkyl, (C₃-C₇)heterocycloalkyl, (C₁-C₆)alkyl-(C₃-C₇)cycloalkyl, (C₁-C₆)alkyl-(C₃-C₇)heterocycloalkyl, aryl, (C₁-C₆)alkyl-aryl, heteroaryl, (C₁-C₆)alkyl-heteroaryl, and N(R⁵)₂;
wherein each (C₁-C₆)alkyl, (C₁-C₆)alkynyl, (C₁-C₆)heteroalkyl, (C₁-C₆)heteroalkynyl, (C₃-C₇)cycloalkyl, (C₃-C₇)heterocycloalkyl, (C₁-C₆)alkyl-(C₃-C₇)cycloalkyl, (C₁-C₆)alkyl-(C₃-C₇)heterocycloalkyl, aryl, (C₁-C₆)alkyl-aryl, heteroaryl, or (C₁-C₆)alkyl-heteroaryl is optionally substituted with deuterium, ¹¹C, ¹⁸F, or ¹²³I.

6. The compound of claim 5, wherein each occurrence of R⁵ is independently selected from the group consisting of hydrogen, (C₁-C₆)alkyl, aryl, (C₁-C₆)alkyl-aryl, heteroaryl, and (C₁-C₆)alkyl-heteroaryl;
wherein each (C₁-C₆)alkyl, aryl, (C₁-C₆)alkyl-aryl, heteroaryl, or (C₁-C₆)alkyl-heteroaryl is optionally substituted with deuterium, ¹¹C, ¹⁸F, or ¹²³I.

7. The compound of claim 6, wherein R is CH₃, ¹¹CH₃, CH₂¹⁸F, (CH₂)₂¹⁸F, CF₃, CF₂¹⁸F, CD₂¹⁸F, 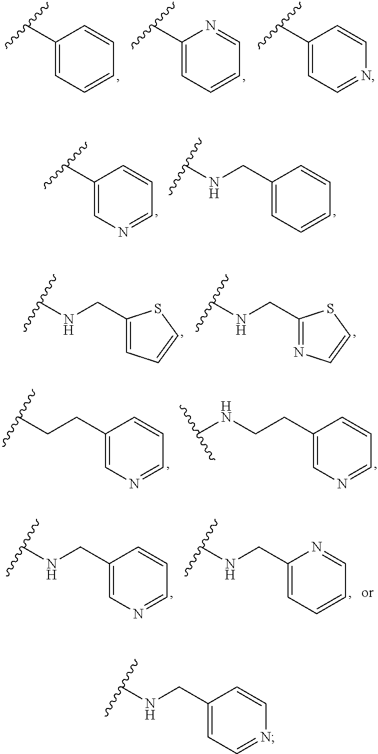
wherein each 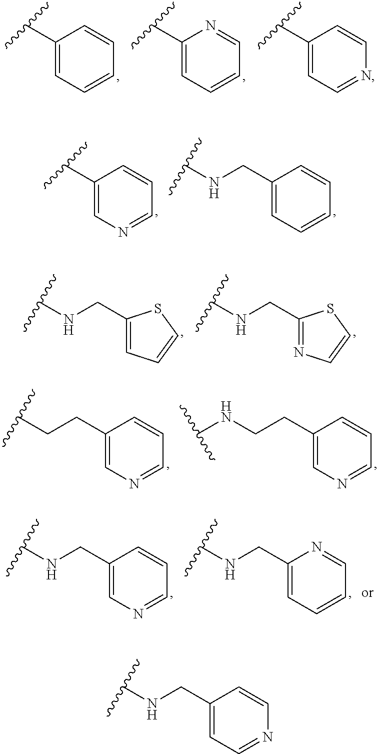
is optionally substituted with deuterium, ¹¹C, ¹⁸F, or ¹²³I.

8. The compound of claim 7, wherein R is CH₃, ¹¹CH₃, or (CH₂)₂¹⁸F.

9. The compound of claim 1, wherein R¹, R², R³, and R⁴ are each independently selected from the group consisting of hydrogen, deuterium, halogen, ¹¹C, ¹⁸F, ¹²³I, (C₁-C₆)alkyl, (C₁-C₆)alkynyl, (C₁-C₆)heteroalkyl, (C₁-C₆)heteroalkynyl, (C₃-C₇)cycloalkyl, (C₃-C₇)heterocycloalkyl, (C₁-C₆)alkyl-(C₃-C₇)cycloalkyl, (C₁-C₆)alkyl-(C₃-C₇)heterocycloalkyl, aryl, (C₁-C₆)alkyl-aryl, heteroaryl, (C₁-C₆)alkyl-heteroaryl, and N(R⁵)₂;
wherein each (C₁-C₆)alkyl, (C₁-C₆)alkynyl, (C₁-C₆)heteroalkyl, (C₁-C₆)heteroalkynyl, (C₃-C₇)cycloalkyl, (C₃-C₇)heterocycloalkyl, (C₁-C₆)alkyl-(C₃-C₇)cycloalkyl, (C₁-C₆)alkyl-(C₃-C₇)heterocycloalkyl, aryl, (C₁-C₆)alkyl-aryl, heteroaryl, or (C₁-C₆)alkyl-heteroaryl is optionally substituted with deuterium, ¹¹C, ¹⁸F, or ¹²³I.

10. The compound of claim 9, wherein each occurrence of R⁵ is independently selected from the group consisting of hydrogen, deuterium, (C₁-C₆)alkyl, aryl, (C₁-C₆)alkyl-aryl, heteroaryl, and (C₁-C₆)alkyl-heteroaryl;
wherein each (C₁-C₆)alkyl, aryl, (C₁-C₆)alkyl-aryl, heteroaryl, or (C₁-C₆)alkyl-heteroaryl is optionally substituted with deuterium, ¹¹C, ¹⁸F, or ¹²³I.

11. The compound of claim 10, wherein R¹, R², R³, and R⁴ are each independently hydrogen, deuterium, halogen, ¹⁸F, ¹²³I, CH₃, ¹¹CH₃, CH₂¹⁸F, (CH₂)₂¹⁸F, CF₃, CF₂¹⁸F, CD₂¹⁸F, 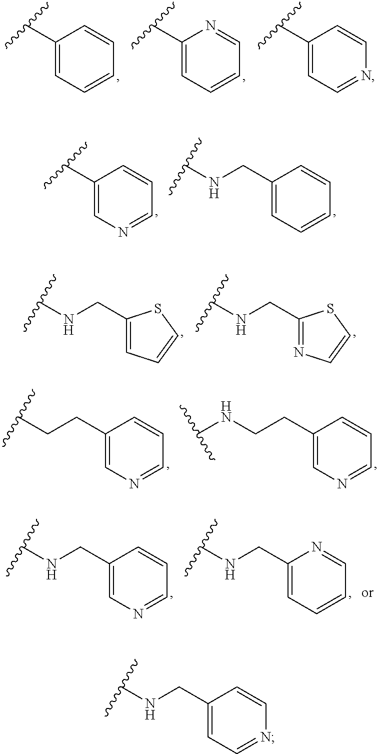
wherein each 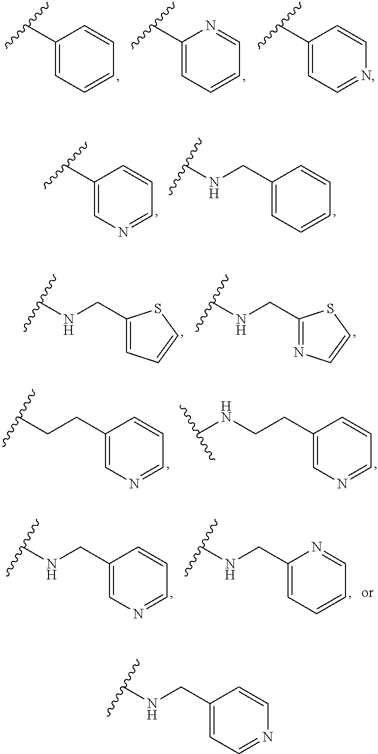
is optionally substituted with deuterium, ¹¹C, ¹⁸F, or ¹²³I.

12. The compound of claim 11, wherein R¹ is hydrogen or ¹⁸F.

13. The compound of claim 11, wherein R² is hydrogen,

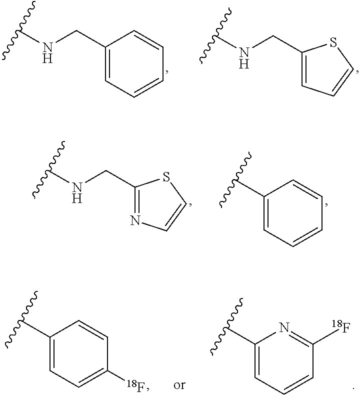 [SMILES: *NCc1ccccc1 ; *NCc1cccs1 ; *NCc1nccs1 ; *c1ccccc1 ; *c1ccc([18F])cc1 ; *c1cccc([18F])n1]

14. The compound of claim 11, wherein R³ is hydrogen or ¹⁸F.

15. The compound of claim 11, wherein R⁴ is halogen, ¹⁸F, CF₂¹⁸F, CH₂¹⁸F, CD₂¹⁸F, CF₃,

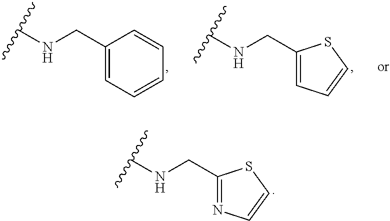 [SMILES: *NCc1ccccc1 ; *NCc1cccs1 ; *NCc1nccs1]

16. The compound of claim 1, wherein:
A is 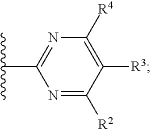
R is CH₃ or ¹¹CH₃;
R¹ and R³ are hydrogen;
R² is N(R⁵)₂;
R⁴ is F, Cl, CF₃, or ¹⁸F; and
each occurrence of R⁵ is independently hydrogen, (C₁-C₆)alkyl-aryl, or (C₁-C₆)alkyl-heteroaryl.

17. The compound of claim 16, wherein each occurrence of R⁵ is independently hydrogen,

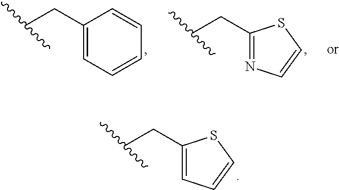 [SMILES: *Cc1ccccc1 ; *Cc1nccs1 ; *Cc1cccs1]

18. The compound of claim 17, wherein R² is

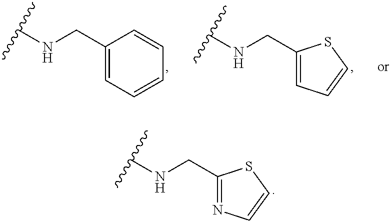 [SMILES: *NCc1ccccc1 ; *NCc1cccs1 ; *NCc1nccs1]

19. The compound of claim 1, wherein:
A is 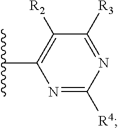
R is CH₃;
R¹ and R² are hydrogen;
R² is ¹⁸F;
R⁴ is (NR⁵)₂; and
each occurrence of R⁵ is independently hydrogen, (C₁-C₆)alkyl-aryl, or (C₁-C₆)alkyl-heteroaryl.

20. The compound of claim 19, wherein each occurrence of R⁵ is independently hydrogen,

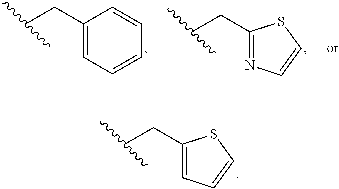 [SMILES: *Cc1ccccc1 ; *Cc1nccs1 ; *Cc1cccs1]

21. The compound of claim 20, wherein R² is

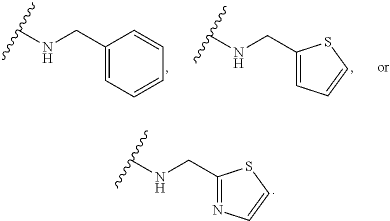 [SMILES: *NCc1ccccc1 ; *NCc1cccs1 ; *NCc1nccs1]

22. The compound of claim 1, wherein:
A is 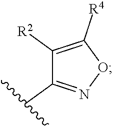
R and R⁴ are (C₁-C₆)alkyl, optionally substituted with one or more deuterium, ¹⁸F, or halogen;
R¹ is hydrogen or ¹⁸F;
R² is aryl, optionally substituted with ¹⁸F, or heteroaryl, optionally substituted with ¹⁸F.

23. The compound of claim 22, wherein R is CH₃ or (CH₂)₂¹⁸F.

24. The compound of claim 22, wherein R² is

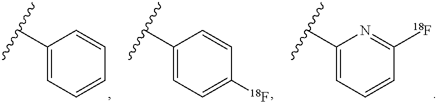

25. The compound of claim 22, wherein R⁴ is CF₃, CF₂¹⁸F, CH₂¹⁸F.

26. The compound of claim 1, wherein the compound of Formula I is 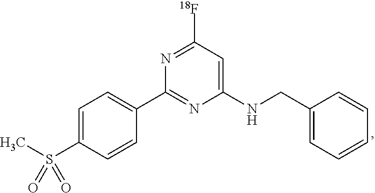 [SMILES: CS(=O)(=O)c1ccc(-c2nc([18F])cc(NCc3ccccc3)n2)cc1]
or a pharmaceutically acceptable salt thereof.

27. The compound of claim 1, wherein the compound of Formula I is 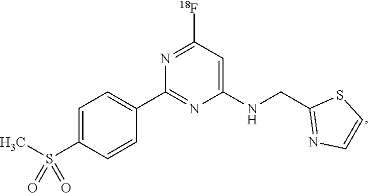 [SMILES: CS(=O)(=O)c1ccc(-c2nc([18F])cc(NCc3nccs3)n2)cc1]
or a pharmaceutically acceptable salt thereof.

28. The compound of claim 1, wherein the compound of Formula I is 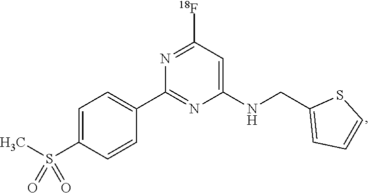 [SMILES: CS(=O)(=O)c1ccc(-c2nc([18F])cc(NCc3cccs3)n2)cc1]
or a pharmaceutically acceptable salt thereof.

29. The compound of claim 1, wherein the compound of Formula I is 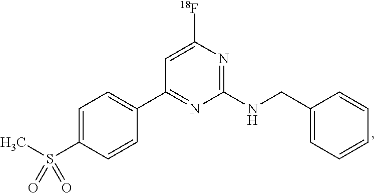 [SMILES: CS(=O)(=O)c1ccc(-c2cc([18F])nc(NCc3ccccc3)n2)cc1]
or a pharmaceutically acceptable salt thereof.

30. The compound of claim 1, wherein the compound of Formula I is 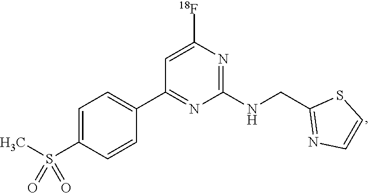 [SMILES: CS(=O)(=O)c1ccc(-c2cc([18F])nc(NCc3nccs3)n2)cc1]
or a pharmaceutically acceptable salt thereof.

31. The compound of claim 1, wherein the compound of Formula I is 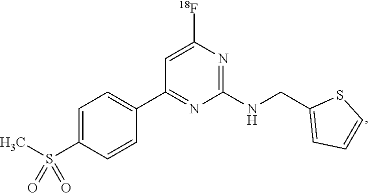 [SMILES: CS(=O)(=O)c1ccc(-c2cc([18F])nc(NCc3cccs3)n2)cc1]
or a pharmaceutically acceptable salt thereof.

32. The compound of claim 1, wherein the compound of Formula I is 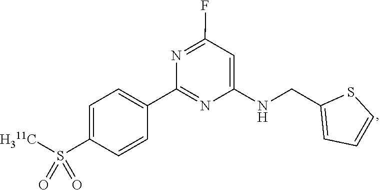 [SMILES: [11CH3]S(=O)(=O)c1ccc(-c2nc(F)cc(NCc3cccs3)n2)cc1]
or a pharmaceutically acceptable salt thereof.

33. The compound of claim 1, wherein the compound of Formula I is 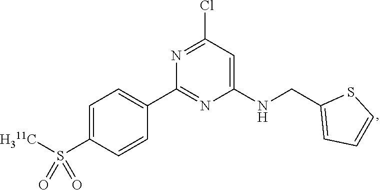 [SMILES: [11CH3]S(=O)(=O)c1ccc(-c2nc(Cl)cc(NCc3cccs3)n2)cc1]
or a pharmaceutically acceptable salt thereof.

34. The compound of claim 1, wherein the compound of Formula I is 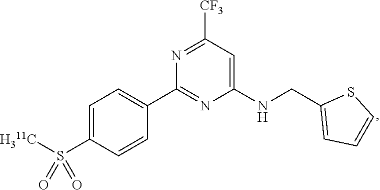 [SMILES: [11CH3]S(=O)(=O)c1ccc(-c2nc(NCc3cccs3)cc(C(F)(F)F)n2)cc1]
or a pharmaceutically acceptable salt thereof.

35. The compound of claim 1, wherein the compound of Formula I is

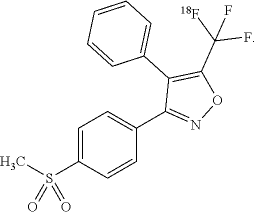 [SMILES: CS(=O)(=O)c1ccc(-c2noc(C(F)(F)[18F])c2-c2ccccc2)cc1]

36. The compound of claim 1, wherein the compound of Formula I is

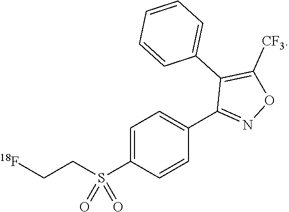 [SMILES: O=S(=O)(CC[18F])c1ccc(-c2noc(C(F)(F)F)c2-c2ccccc2)cc1]

37. The compound of claim 1, wherein the compound of Formula I is

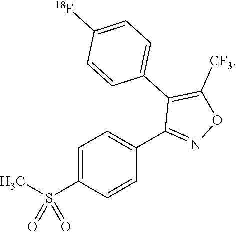 [SMILES: CS(=O)(=O)c1ccc(-c2noc(C(F)(F)F)c2-c2ccc([18F])cc2)cc1]

38. The compound of claim 1, wherein the compound of Formula I is

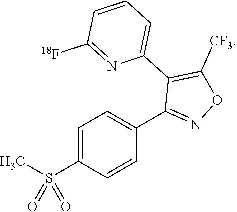 [SMILES: CS(=O)(=O)c1ccc(-c2noc(C(F)(F)F)c2-c2cccc([18F])n2)cc1]

39. The compound of claim 1, wherein the compound of Formula I is

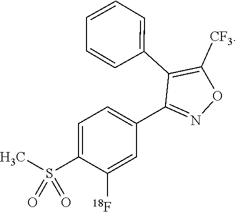 [SMILES: CS(=O)(=O)c1ccc(-c2noc(C(F)(F)F)c2-c2ccccc2)cc1[18F]]

40. The compound of claim 1, wherein the compound of Formula I is

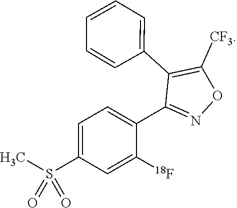 [SMILES: CS(=O)(=O)c1ccc(-c2noc(C(F)(F)F)c2-c2ccccc2)c([18F])c1]

41. The compound of claim 1, wherein the compound of Formula I is

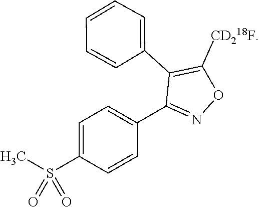

42. The compound of claim 1, wherein the compound of Formula I is [chemical structure]
